(19) United States
(12) Patent Application Publication
(10) Pub. No.: US 2015/0157739 A1
(43) Pub. Date: Jun. 11, 2015

(54) Imidazo[2,1]thiazol-3-one derivatives

(71) Applicant: Hoffmann-La Roche Inc., Little Falls, NJ (US)

(72) Inventors: Luca Gobbi, Muttenz (CH); Henner Knust, Rheinfelden (DE); Andreas Koblet, Bottmingen (CH)

(73) Assignee: HOFFMANN-LA ROCHE INC., Little Falls, NJ (US)

(21) Appl. No.: 14/621,903

(22) Filed: Feb. 13, 2015

Related U.S. Application Data
(63) Continuation of application No. PCT/EP2013/066447, filed on Aug. 6, 2013.

(30) Foreign Application Priority Data
Aug. 14, 2012 (EP) 12180367.0

(51) Int. Cl. A61K 49/00; C07D 513/04

(52) U.S. Cl. CPC A61K 49/0004; C07D 513/04

(57) ABSTRACT

The invention relates to imidazo[2,1-b]thiazol-3-one derivatives of formula

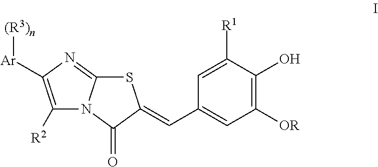

wherein the variables are defined herein,
or to a pharmaceutically acceptable acid addition salt, to a racemic mixture or to its corresponding enantiomer and/or optical isomers thereof.

It has been shown that the present compounds may be used for binding and imaging tau aggregates and related b-sheet aggregates including besides others beta-amyloid aggregates or alpha-synuclein aggregates, especially for use in binding and imaging tau aggregates in Alzheimer's patients.

This application is a continuation of International Application No. PCT/EP2013/066447, filed Aug. 6, 2013, which claims priority to EP 12180367.0, filed Aug. 14, 2012, each of which is incorporated herein by reference in its entirety.

The invention relates to imidazo[2,1-b]thiazol-3-one derivatives of formula

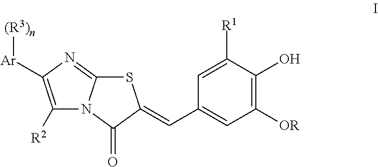

wherein
Ar is phenyl, pyridinyl, 2,3-dihydro-benzo[1,4]dioxinyl, 1,3-dihydro-indol-2-one, pyrazinyl, isoxazol-3-yl, imidazolyl, thiophenyl or pyrimidinyl;
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
R³ is hydrogen, halogen, lower alkyl, lower alkyl substituted by halogen, lower alkoxy, lower alkoxy substituted by halogen, O(CH₂)ₘO(CH₂)ₘO-lower alkyl substituted by halogen, cyano, lower alkoxy substituted by hydroxy, lower alkenyloxy, C(O)OH, heterocycloalkyl selected from morpholinyl, pyrrolidinyl or pyrrolidin-2-one, or is heteroaryl selected from imidazolyl substituted by lower alkyl, or is
   NR′R″ and R′/R″ are independently from each other hydrogen or lower alkyl or —C(O)-lower alkyl; or is
   —C(O)NR⁴R⁵ and
   R⁴ is hydrogen or lower alkyl and
   R⁵ is hydrogen, lower alkyl, lower alkenyl, —(CH₂)ₘO-lower alkyl substituted by halogen, lower alkyl substituted by halogen, —(CH₂)ₙ-phenyl optionally substituted by halogen, —CH₂)ₘNHC(O)-lower alkyl, or —(CH₂)ₘNH₂, or
   R⁴ and R⁵ may form together with the N-atom to which they are attached a piperidine or azetidine ring, which may be substituted by halogen; or is
   —C(O)O-lower alkyl substituted by halogen;
n is 1 or 2;
m is 1, 2 or 3;
   or to a pharmaceutically acceptable acid addition salt, to a racemic mixture or to its corresponding enantiomer and/or optical isomers thereof.

The most similar compound imidazo[2,1-b]thiazol-3(2H)-one, 2-[(4-hydroxy-3-methoxyphenyl)methylene]-5-phenyl- is specifically disclosed in Journal of the Indian Chemical Society (1981), 58(11), 1117-18.

It has been shown that the present compounds may be used for binding and imaging tau aggregates and related b-sheet aggregates including besides others beta-amyloid aggregates or alpha-synuclein aggregates, especially for use in binding and imaging tau aggregates in Alzheimer patients.

Alzheimer's disease (AD) is a progressive neurodegenerative disorder characterized by cognitive decline, irreversible memory loss, disorientation and language impairment (Arch. Neurol. 1985, 42(11), 1097-1105). Postmortem examination of AD brain sections reveals abundant senile plaques (SPs), composed of beta amyloid (Aβ) peptides, and numerous neurofibrillary tangles (NFTs) formed by filaments of hyperphosphorylated tau protein.

Tau belongs to the family of microtubule-associated proteins and is mainly expressed in neurons where it plays an important role in the assembly of tubulin monomers into microtubules to constitute the neuronal microtubule network as tracks for axonal transport (Brain Res. Rev. 2000, 33(1), 95-130). Tau is translated from a single gene located on chromosome 17 and the expression is developmentally regulated by an alternative splicing mechanism generating six different isoforms in the human adult brain that can be distinguished by their number of binding domains. The underlying mechanisms leading to tau hyperphosphorylation, misfolding and aggregation are not well understood, but the deposition of tau aggregates follows a stereotyped spatiotemporal pathway both at the intracellular levels as well as on the level of brain topography.

The recent discovery of tau gene mutations leading to frontotemporal dementia (FTD) with parkinsonism linked to chromosome 17 has reinforced the predominant role attributed to tau in the pathogenesis of neurodegenerative disorders and underlined the fact that distinct sets of tau isoforms expressed in different neuronal populations could lead to different pathologies (Biochim. Biophys. Acta 2005, 1739(2) 240-250). Neurodegenerative diseases characterized by pathological tau accumulation are termed ‘tauopathies’ (Ann. Rev. Neurosci. 2001, 24, 1121-1159). Besides AD and FTD, other tauopathies include progressive supranuclear palsy (PSP), tangle-predominant dementia, Pick's disease, frontotemporal lobar degeneration (FTLD), Down's syndrome and others.

A direct correlation has been established between the progressive involvement of neocortical areas and the increasing severity of dementia, suggesting that pathological tau aggregates such as NFTs are a reliable marker of the neurodegenerative process. The degree of NFT involvement in AD is defined by Braak stages (Acta Neuropathol. 1991, 82, 239-259). Braak stages I and II are defined when NFT involvement is confined mainly to the transentorhinal region of the brain, stages III and IV are diagnosed when limbic regions such as the hippocampus are involved, and stages V and VI when extensive neocortical involvement is found.

Presently, detection of tau aggregates is only possible by histological analysis of biopsy or autopsy materials. In vivo imaging of tau pathology would provide novel insights into deposition of tau aggregates in the human brain and allow to non-invasively examine the degree of tau pathology, quantify changes in tau deposition over time, assess its correlation with cognition and analyze the efficacy of an anti-tau therapy. Potential ligands for detecting tau aggregates in the living brain must cross the blood-brain barrier and possess high affinity and specificity for tau aggregates. To this end, successful neuroimaging radiotracers must have appropriate lipophilicity (log D 1-3) and low molecular weight (<450), show rapid clearance from blood and low non-specific binding.

Therefore, the object of the present application is to find an imaging tool which will improve diagnosis by identifying potential patients with excess of tau aggregates in the brain, which may be likely to develop Alzheimer's disease. It will also be useful to monitor the progression of the disease. When an anti-tau aggregate drug become available, imaging tau tangles in the brain may provide a essential tool for monitoring treatment.

A further object of the present invention is a method of imaging tau aggregate deposits, comprising
introducing into a mammal a detectable quantity of a composition
allowing sufficient time for the compound of formula I to be associated with tau aggregate deposits, and
detecting the compound associated with one or more tau aggregate deposits.
   A further object of the present invention is a pharmaceutical composition, containing compounds of formula I and pharmaceutical acceptable carriers, which may be used for identifying potential patients.

The following definitions of the general terms used in the present description apply irrespective of whether the terms in question appear alone or in combination.

As used herein, the term “lower alkyl” denotes a saturated, i.e. aliphatic hydrocarbon group including a straight or branched carbon chain with 1-7 carbon atoms. Examples for “alkyl” are methyl, ethyl, n-propyl, and isopropyl.

The term “alkoxy” denotes a group —O—R′ wherein R′ is lower alkyl as defined above.

The term “halogen” denotes chlorine, bromine, fluorine or iodine.

The term “lower alkyl substituted by halogen” denotes an alkyl group as defined above, wherein at least one hydrogen atom is replaced by a halogen atom.

The term “lower alkoxy substituted by halogen” denotes an alkoxy group as defined above, wherein at least one hydrogen atom is replaced by a halogen atom.

The term “lower alkoxy substituted by hydroxy” denotes an alkoxy group as defined above, wherein at least one hydrogen atom is replaced by a hydroxy group.

The term “lower alkenyl” denotes an unsaturated hydrocarbon group, containing from 2 to 7 carbon atoms.

The term “lower alkenyloxy” denotes the group —O—R″ wherein R″ is lower alkenyl as defined above.

The term “pharmaceutically acceptable salt” or “pharmaceutically acceptable acid addition salt” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

It has been found that the compounds of formula I may be used for binding and imaging tau aggregates and related b-sheet aggregates including besides others beta-amyloid aggregates or alpha-synuclein aggregates.

One object of the present invention are compounds of formula IA

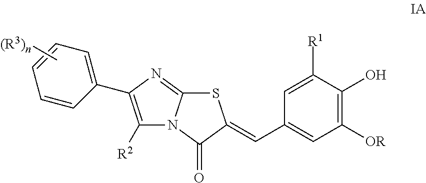

wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
R³ is hydrogen, halogen, lower alkyl, lower alkyl substituted by halogen, lower alkoxy, lower alkoxy substituted by halogen, O(CH₂)ₘO(CH₂)ₘO-lower alkyl substituted by halogen, cyano, lower alkoxy substituted by hydroxy, lower alkenyloxy, C(O)OH, heterocycloalkyl selected from morpholinyl, pyrrolidinyl or pyrrolidin-2-one, or is heteroaryl selected from imidazolyl substituted by lower alkyl, or is NR′R″ and R′/R″ are independently from each other hydrogen or lower alkyl or
   —C(O)-lower alkyl; or is
   —C(O)NR⁴R⁵ and
   R⁴ is hydrogen or lower alkyl and
   R⁵ is hydrogen, lower alkyl, lower alkenyl, —(CH₂)ₘO-lower alkyl substituted by halogen, lower alkyl substituted by halogen, —(CH₂)ₙ-phenyl optionally substituted by halogen, —CH₂)ₘNHC(O)-lower alkyl, or —(CH₂)ₘNH₂, or
   R⁴ and R⁵ may form together with the N-atom to which they are attached a piperidine or azetidine ring, which may be substituted by halogen; or is
   —C(O)O-lower alkyl substituted by halogen;
n is 1 or 2;
m is 1, 2 or 3;
   or a pharmaceutically acceptable acid addition salt, a racemic mixture or its corresponding enantiomer and/or optical isomers thereof,
   for example the following compounds:
6-(4-Chloro-phenyl)-2-[1-(4-hydroxy-3,5-dimethoxy-phenyl)-meth-(Z)-ylidene]-imidazo[2,1-b]thiazol-3-one
6-(4-Chloro-phenyl)-2-[1-(4-hydroxy-3-methoxy-phenyl)-meth-(Z)-ylidene]-imidazo[2,1-b]thiazol-3-one
6-(4-Chloro-phenyl)-2-[1-(3-fluoro-4-hydroxy-5-methoxy-phenyl)-meth-(Z)-ylidene]-imidazo[2,1-b]thiazol-3-one
6-(4-Chloro-phenyl)-2-[1-(3-ethoxy-4-hydroxy-phenyl)-meth-(Z)-ylidene]-imidazo[2,1-b]thiazol-3-one
6-(4-Chloro-phenyl)-2-[1-(3-chloro-4-hydroxy-5-methoxy-phenyl)-meth-(Z)-ylidene]-imidazo[2,1-b]thiazol-3-one
6-(4-Chloro-phenyl)-2-[1-(3-bromo-4-hydroxy-5-methoxy-phenyl)-meth-(Z)-ylidene]-imidazo[2,1-b]thiazol-3-one
6-(4-Chloro-phenyl)-2-[1-(3,4-dihydroxy-5-methoxy-phenyl)-meth-(Z)-ylidene]-imidazo[2,1-b]thiazol-3-one
2-[1-(3-Fluoro-4-hydroxy-5-methoxy-phenyl)-meth-(Z)-ylidene]-6-o-tolyl-imidazo[2,1-b]thiazol-3-one
2-[1-(3-Chloro-4-hydroxy-5-methoxy-phenyl)-meth-(Z)-ylidene]-6-o-tolyl-imidazo[2,1-b]thiazol-3-one
2-[1-(4-hydroxy-3-methoxy-phenyl)-meth-(Z)-ylidene]-6-o-tolyl-imidazo[2,1-b]thiazol-3-one
2-[1-(3-Chloro-4-hydroxy-5-methoxy-phenyl)-meth-(Z)-ylidene]-6-(4-methoxy-phenyl)-5-methyl-imidazo[2,1-b]thiazol-3-one
2-[1-(3-Fluoro-4-hydroxy-5-methoxy-phenyl)-meth-(Z)-ylidene]-6-(4-methoxy-phenyl)-5-methyl-imidazo[2,1-b]thiazol-3-one
2-[1-(4-Hydroxy-3,5-dimethoxy-phenyl)-meth-(Z)-ylidene]-6-(3-trifluoromethoxy-phenyl)-imidazo[2,1-b]thiazol-3-one
2-[1-(4-Hydroxy-3,5-dimethoxy-phenyl)-meth-(Z)-ylidene]-6-(3-trifluoromethyl-phenyl)-imidazo[2,1-b]thiazol-3-one
2-[1-(4-Hydroxy-3,5-dimethoxy-phenyl)-meth-(Z)-ylidene]-6-m-tolyl-imidazo[2,1-b]thiazol-3-one
6-(3-Chloro-phenyl)-2-[1-(4-hydroxy-3,5-dimethoxy-phenyl)-meth-(Z)-ylidene]-imidazo[2,1-b]thiazol-3-one
2-[1-(3-Chloro-4-hydroxy-5-methoxy-phenyl)-meth-(Z)-ylidene]-6-(3-trifluoromethoxy-phenyl)-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(2-fluoro-ethoxy)-ethyl]-N-methyl-benzamide
2-[1-(3-Chloro-4-hydroxy-5-methoxy-phenyl)-meth-(Z)-ylidene]-6-(3-trifluoromethyl-phenyl)-imidazo[2,1-b]thiazol-3-one
6-(3-Chloro-phenyl)-2-[1-(3-chloro-4-hydroxy-5-methoxy-phenyl)-meth-(Z)-ylidene]-imidazo[2,1-b]thiazol-3-one
6-(4-Chloro-phenyl)-2-[1-(3-ethoxy-4-hydroxy-5-methoxy-phenyl)-meth-(Z)-ylidene]-imidazo[2,1-b]thiazol-3-one
6-(4-Chloro-phenyl)-2-[1-(3-(2-fluoro-ethoxy)-4-hydroxy-5-methoxy-phenyl)-meth-(Z)-ylidene]-imidazo[2,1-b]thiazol-3-one
2-[1-(3-Chloro-4-hydroxy-5-methoxy-phenyl)-meth-(Z)-ylidene]-6-(3-fluoro-4-trifluoromethyl-phenyl)-imidazo[2,1-b]thiazol-3-one
2-[1-(3-Hydroxy-3,5-dimethoxy-phenyl)-meth-(Z)-ylidene]-6-(3-fluoro-4-trifluoromethyl-phenyl)-imidazo[2,1-b]thiazol-3-one
6-(4-Chloro-phenyl)-2-[1-(4-hydroxy-3-isopropoxy-5-methoxy-phenyl)-meth-(Z)-ylidene]-imidazo[2,1-b]thiazol-3-one
6-(4-Chloro-phenyl)-2-[1-(4-hydroxy-3-methoxy-5-propoxy-phenyl)-meth-(Z)-ylidene]-imidazo[2,1-b]thiazol-3-one
6-(4-Chloro-phenyl)-2-[1-(4-hydroxy-3-methoxy-5-methyl-phenyl)-meth-(Z)-ylidene]-imidazo[2,1-b]thiazol-3-one
2-[1-(4-Hydroxy-3-methoxy-5-propoxy-phenyl)-meth-(Z)-ylidene]-6-(3-trifluoromethoxy-phenyl)-imidazo[2,1-b]thiazol-3-one
2-[1-(4-Hydroxy-3-methoxy-5-isopropoxy-phenyl)-meth-(Z)-ylidene]-6-(3-trifluoromethoxy-phenyl)-imidazo[2,1-b]thiazol-3-one
2-[1-(3-Fluoro-4-hydroxy-5-methoxy-phenyl)-meth-(Z)-ylidene]-6-(3-trifluoromethoxy-phenyl)-imidazo[2,1-b]thiazol-3-one
2-[1-(4-Hydroxy-3-methoxy-phenyl)-meth-(Z)-ylidene]-6-(3-trifluoromethoxy-phenyl)-imidazo[2,1-b]thiazol-3-one
2-[1-(3-Bromo-4-hydroxy-5-methoxy-phenyl)-meth-(Z)-ylidene]-6-(3-trifluoromethoxy-phenyl)-imidazo[2,1-b]thiazol-3-one
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzonitrile
2-[1-(3-fluoro-4-hydroxy-5-methoxy-phenyl)-meth-(Z)-ylidene]-6-(3-chloro-phenyl)-imidazo[2,1-b]thiazol-3-one
2-[1-(3-Ethyl-4-hydroxy-5-methoxy-phenyl)-meth-(Z)-ylidene]-6-(3-trifluoromethoxy-phenyl)-imidazo[2,1-b]thiazol-3-one
2-[1-(3-fluoro-4-hydroxy-5-methoxy-phenyl)-meth-(Z)-ylidene]-6-(3-(trifluoromethyl)-phenyl)-imidazo[2,1-b]thiazol-3-one
2-[1-(3-fluoro-4-hydroxy-5-methoxy-phenyl)-meth-(Z)-ylidene]-6-(4-fluoro-3-(trifluoromethyl)-phenyl)-imidazo[2,1-b]thiazol-3-one
2-[1-(3-fluoro-4-hydroxy-5-methoxy-phenyl)-meth-(Z)-ylidene]-6-phenyl-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzonitrile
3-{2-[1-(3-Bromo-4-hydroxy-5-methoxy-phenyl)-meth-(Z)-ylidene]-3-oxo-2,3-dihydro-imidazo[2,1-b]thiazol-6-yl}-benzonitrile
2-[1-(3-bromo-4-hydroxy-5-methoxy-phenyl)-meth-(Z)-ylidene]-6-(4-fluoro-3-(trifluoromethyl)-phenyl)-imidazo[2,1-b]thiazol-3-one
2-[1-(3-Fluoro-4-hydroxy-5-methoxy-phenyl)-meth-(Z)-ylidene]-6-(3-methoxy-phenyl)-imidazo[2,1-b]thiazol-3-one
2-[1-(3-Chloro-4-hydroxy-5-methoxy-phenyl)-meth-(Z)-ylidene]-6-(3,5-difluoro-phenyl)-imidazo[2,1-b]thiazol-3-one
3-{2-[1-(4-Hydroxy-3,5-dimethoxy-phenyl)-meth-(Z)-ylidene]-3-oxo-2,3-dihydro-imidazo[2,1-b]thiazol-6-yl}-benzonitrile
3-{2-[1-(4-Hydroxy-3-methoxy-5-isopropoxyphenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-m-tolyl-imidazo[2,1-b]thiazol-3-one
2-[1-(3-Chloro-4-hydroxy-5-methoxy-phenyl)-meth-(Z)-ylidene]-6-(2-fluoro-phenyl)-imidazo[2,1-b]thiazol-3-one
2-[1-(3-Chloro-4-hydroxy-5-methoxy-phenyl)-meth-(Z)-ylidene]-6-(3-ethyl-phenyl)-imidazo[2,1-b]thiazol-3-one
2-[1-(3-Chloro-4-hydroxy-5-methoxy-phenyl)-meth-(Z)-ylidene]-6-(2-methoxy-phenyl)-imidazo[2,1-b]thiazol-3-one
2-[1-(3-Chloro-4-hydroxy-5-methoxy-phenyl)-meth-(Z)-ylidene]-6-(2,5-difluoro-phenyl)-imidazo[2,1-b]thiazol-3-one
2-[1-(3-Chloro-4-hydroxy-5-methoxy-phenyl)-meth-(Z)-ylidene]-6-(3-fluoro-phenyl)-imidazo[2,1-b]thiazol-3-one
2-[1-(3-Fluoro-4-hydroxy-5-methoxy-phenyl)-meth-(Z)-ylidene]-6-(3-fluoro-phenyl)-imidazo[2,1-b]thiazol-3-one
2-[1-(4-Hydroxy-3,5-dimethoxy-phenyl)-meth-(Z)-ylidene]-6-(3-fluoro-phenyl)-imidazo[2,1-b]thiazol-3-one
2-[1-(3-Chloro-4-hydroxy-5-methoxy-phenyl)-meth-(Z)-ylidene]-6-phenyl-imidazo[2,1-b]thiazol-3-one
2-[1-(3-Chloro-4-hydroxy-5-methoxy-phenyl)-meth-(Z)-ylidene]-6-(3-methoxy-phenyl)-imidazo[2,1-b]thiazol-3-one
2-[1-(3-Chloro-4-hydroxy-5-methoxy-phenyl)-meth-(Z)-ylidene]-6-(3-(difluoromethoxy)-phenyl)-imidazo[2,1-b]thiazol-3-one
(Z)-3-(2-(3-fluoro-4-hydroxy-5-methoxybenzylidene)-3-oxo-2,3-dihydroimidazo[2,1-b]thiazol-5-yl)benzonitrile
2-[1-(3-Chloro-4-hydroxy-5-methoxy-phenyl)-meth-(Z)-ylidene]-6-(3-(chloromethyl)phenyl)-imidazo[2,1-b]thiazol-3-one
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-(3-(2-fluoroethoxyl)phenyl)-imidazo[2,1-b]thiazol-3-one
2-[1-(3-Chloro-4-hydroxy-5-methoxy-phenyl)-meth-(Z)-ylidene]-6-(4-(2-fluoroethoxyl)phenyl)-imidazo[2,1-b]thiazol-3-one
2-[1-(3-Chloro-4-hydroxy-5-methoxy-phenyl)-meth-(Z)-ylidene]-6-(2-(2-fluoroethoxyl)phenyl)-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-(4-(difluoromethoxy)phenyl)-imidazo[2,1-b]thiazol-3-one
2-[1-(3-Chloro-4-hydroxy-5-methoxy-phenyl)-meth-(Z)-ylidene]-6-(3-morpholinophenyl)-imidazo[2,1-b]thiazol-3-one
2-[1-(3-Chloro-4-hydroxy-5-methoxy-phenyl)-meth-(Z)-ylidene]-6-(4-(pyrrolidin-1-yl)phenyl)-imidazo[2,1-b]thiazol-3-one
2-[1-(3-Chloro-4-hydroxy-5-methoxy-phenyl)-meth-(Z)-ylidene]-6-(4-(diethylamino)phenyl)-imidazo[2,1-b]thiazol-3-one
2-[1-(3-Chloro-4-hydroxy-5-methoxy-phenyl)-meth-(Z)-ylidene]-6-(4-morpholinophenyl)-imidazo[2,1-b]thiazol-3-one
2-[1-(3-Chloro-4-hydroxy-5-methoxy-phenyl)-meth-(Z)-ylidene]-6-(4-(dimethylamino)phenyl)-imidazo[2,1-b]thiazol-3-one
2-[1-(3-Chloro-4-hydroxy-5-methoxy-phenyl)-meth-(Z)-ylidene]-6-[3-(2-oxo-pyrrolidin-1-yl)-phenyl]-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid
2-[1-(3-Chloro-4-hydroxy-5-methoxy-phenyl)-meth-(Z)-ylidene]-6-(3,4-dimethoxy-phenyl)-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzamide
N-(4-{2-[1-(3-Chloro-4-hydroxy-5-methoxy-phenyl)-meth-(Z)-ylidene]-3-oxo-2,3-dihydro-imidazo[2,1-b]thiazol-6-yl}-phenyl)-acetamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid
2-[1-(3-Chloro-4-hydroxy-5-methoxy-phenyl)-meth-(Z)-ylidene]-6-(2,6-difluoro-phenyl)-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-propyl-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-benzamide
N-Allyl-3-{2-[1-(3-chloro-4-hydroxy-5-methoxy-phenyl)-meth-(Z)-ylidene]-3-oxo-2,3-dihydro-imidazo[2,1-b]thiazol-6-yl}-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-propyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoro-propyl)-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(3-fluoro-phenyl)-ethyl]-N-methyl-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-N-methyl-benzamide
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-N-methyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N,N-dimethyl-benzamide
2-[1-(3-Chloro-4-hydroxy-5-methoxy-phenyl)-meth-(Z)-ylidene]-6-[4-(4-fluoro-piperidine-1-carbonyl)-phenyl]-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-N-propyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoropropoxy)-benzamide
2-[1-(3-Chloro-4-hydroxy-5-methoxy-phenyl)-meth-(Z)-ylidene]-6-[4-(3-fluoro-azetidine-1-carbonyl)-phenyl]-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoropropoxy)-N-methyl-benzamide
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzoic acid
6-[4-(3-Fluoro-azetidine-1-carbonyl)-phenyl]-2-[1-(3-fluoro-4-hydroxy-5-methoxy-phenyl)-meth-(Z)-ylidene]-imidazo[2,1-b]thiazol-3-one
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoroethoxy)-benzamide
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(3-fluoropropoxy)-N-methyl-benzamide
2-[1-(3-Chloro-4-hydroxy-5-methoxy-phenyl)-meth-(Z)-ylidene]-6-[3-(3-fluoro-azetidine-1-carbonyl)-phenyl]-imidazo[2,1-b]thiazol-3-one
2-[1-(3-Chloro-4-hydroxy-5-methoxy-phenyl)-meth-(Z)-ylidene]-6-[3-(4-fluoro-piperidine-1-carbon-phenyl]-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(2-fluoro-ethoxy)-ethyl]-N-methyl-benzamide
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoroethoxy)-N-methyl-benzamide
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoropropoxy)-N-methyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid 2-fluoro-ethyl ester
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid 3-fluoro-propyl ester
[2-(4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoylamino)-ethyl]-carbamic acid tert-butyl ester
N-(2-Amino-ethyl)-4-{2-[1-(3-chloro-4-hydroxy-5-methoxy-phenyl)-meth-(Z)-ylidene]-3-oxo-2,3-dihydro-imidazo[2,1-b]thiazol-6-yl}-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzenesulfonyl fluoride
3-{2-[1-[3-Chloro-5-(3-fluoro-propoxy)-4-hydroxy-phenyl]-meth-(Z)-ylidene]-3-oxo-2,3-dihydro-imidazo[2,1-b]thiazol-6-yl}-benzonitrile
3-Bromo-5-{2-[1-(3-chloro-4-hydroxy-5-methoxy-phenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-(3-{2-[2-(2-fluoro-ethoxy)-ethoxy]-ethoxy}-phenyl)-imidazo[2,1-b]thiazol-3-one or
2-[1-(3-Chloro-4-hydroxy-5-methoxy-phenyl)-meth-(Z)-ylidene]-6-(2-{2-[2-(2-fluoro-ethoxy)-ethoxy]-ethoxy}-phenyl)-imidazo[2,1-b]thiazol-3-one.

One further embodiment of the invention are compounds of formula IB

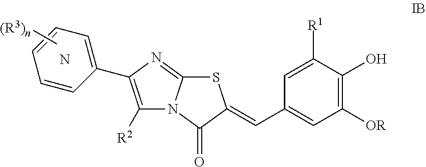

wherein the N-atom of the pyridinyl group may be in different positions, and wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
R³ is hydrogen, halogen, lower alkyl, lower alkyl substituted by halogen, lower alkoxy, lower alkoxy substituted by halogen, O(CH₂)ₘO(CH₂)ₘO-lower alkyl substituted by halogen, cyano, lower alkoxy substituted by hydroxy, lower alkenyloxy, C(O)OH, heterocycloalkyl selected from morpholinyl, pyrrolidinyl or pyrrolidin-2-one, or is heteroaryl selected from imidazolyl substituted by lower alkyl, or is
   NR′R″ and R′/R″ are independently from each other hydrogen or lower alkyl or —C(O)-lower alkyl; or is
   —C(O)NR⁴R⁵ and
   R⁴ is hydrogen or lower alkyl and
   R⁵ is hydrogen, lower alkyl, lower alkenyl, —(CH₂)ₘO-lower alkyl substituted by halogen, lower alkyl substituted by halogen, —(CH₂)ₙ-phenyl optionally substituted by halogen, —CH₂)ₘNHC(O)-lower alkyl, or —(CH₂)ₘNH₂, or
   R⁴ and R⁵ may form together with the N-atom to which they are attached a piperidine or azetidine ring, which may be substituted by halogen; or is
   —C(O)O-lower alkyl substituted by halogen;
n is 1 or 2;
m is 1, 2 or 3;
   or a pharmaceutically acceptable acid addition salt, a racemic mixture or its corresponding enantiomer and/or optical isomers thereof, for example
2-[1-(4-Hydroxy-3,5-dimethoxy-phenyl)-meth-(Z)-ylidene]-6-pyridin-4-yl-imidazo[2,1-b]thiazol-3-one
2-[1-(4-Hydroxy-3-methoxy-phenyl)-meth-(Z)-ylidene]-6-(2-methyl-pyridin-4-yl)-imidazo[2,1-b]thiazol-3-one
2-[1-(4-Hydroxy-3-methoxy-phenyl)-meth-(Z)-ylidene]-6-(2,6-dimethyl-pyridin-4-yl)-imidazo[2,1-b]thiazol-3-one
2-[1-(4-Hydroxy-3,5-dimethoxy-phenyl)-meth-(Z)-ylidene]-6-(2-methyl-pyridin-4-yl)-imidazo[2,1-b]thiazol-3-one
2-[1-(4-Hydroxy-3,5-dimethoxy-phenyl)-meth-(Z)-ylidene]-6-(2,6-dimethyl-pyridin-4-yl)-imidazo[2,1-b]thiazol-3-one
2-[1-(3-Fluoro-4-hydroxy-5-methoxy-phenyl)-meth-(Z)-ylidene]-6-(6-methoxy-pyridin-2-yl)-imidazo[2,1-b]thiazol-3-one
2-[1-(3-Chloro-4-hydroxy-5-methoxy-phenyl)-meth-(Z)-ylidene]-6-(2,6-dimethyl-pyridin-4-yl)-imidazo[2,1-b]thiazol-3-one
2-[1-(3-Fluoro-4-hydroxy-5-methoxy-phenyl)-meth-(Z)-ylidene]-6-(2,6-dimethyl-pyridin-4-yl)-imidazo[2,1-b]thiazol-3-one
2-[1-(3-Bromo-4-hydroxy-5-methoxy-phenyl)-meth-(Z)-ylidene]-6-(2,6-dimethyl-pyridin-4-yl)-imidazo[2,1-b]thiazol-3-one
2-[1-(3-Chloro-4-hydroxy-5-methoxy-phenyl)-meth-(Z)-ylidene]-6-(pyridin-4-yl)-imidazo[2,1-b]thiazol-3-one
2-[1-(3-Fluoro-4-hydroxy-5-methoxy-phenyl)-meth-(Z)-ylidene]-6-(pyridin-4-yl)-imidazo[2,1-b]thiazol-3-one
2-[1-(3-Bromo-4-hydroxy-5-methoxy-phenyl)-meth-(Z)-ylidene]-6-(pyridin-4-yl)-imidazo[2,1-b]thiazol-3-one
2-[1-(3-Fluoro-4-hydroxy-5-methoxy-phenyl)-meth-(Z)-ylidene]-6-(pyridin-3-yl)-imidazo[2,1-b]thiazol-3-one
2-[1-(3-Bromo-4-hydroxy-5-methoxy-phenyl)-meth-(Z)-ylidene]-6-(pyridin-3-yl)-imidazo[2,1-b]thiazol-3-one
2-[1-(3-Chloro-4-hydroxy-5-methoxy-phenyl)-meth-(Z)-ylidene]-6-(2-methylpyridin-4-yl)-imidazo[2,1-b]thiazol-3-one
2-[1-(3-Fluoro-4-hydroxy-5-methoxy-phenyl)-meth-(Z)-ylidene]-6-(2-methylpyridin-4-yl)-imidazo[2,1-b]thiazol-3-one
2-[1-(3-Bromo-4-hydroxy-5-methoxy-phenyl)-meth-(Z)-ylidene]-6-(2-methylpyridin-4-yl)-imidazo[2,1-b]thiazol-3-one
2-[1-(3-Chloro-4-hydroxy-5-methoxy-phenyl)-meth-(Z)-ylidene]-6-(pyridin-3-yl)-imidazo[2,1-b]thiazol-3-one
2-[1-(3-Chloro-4-hydroxy-5-methoxy-phenyl)-meth-(Z)-ylidene]-6-[6-(4-methyl-3H-imidazol-1-yl)-pyridin-3-yl]-imidazo[2,1-b]thiazol-3-one
2-[1-(3-Chloro-4-hydroxy-5-propoxy-phenyl)-meth-(Z)-ylidene]-6-(4-methyl-pyridin-3-yl)-imidazo[2,1-b]thiazol-3-one
2-[1-(3-Chloro-4-hydroxy-5-propoxy-phenyl)-meth-(Z)-ylidene]-6-pyridin-3-yl-imidazo[2,1-b]thiazol-3-one
2-[1-(3-Chloro-4-hydroxy-5-propoxy-phenyl)-meth-(Z)-ylidene]-6-pyridin-4-yl-imidazo[2,1-b]thiazol-3-one
2-[1-(3-Chloro-4-hydroxy-5-propoxy-phenyl)-meth-(Z)-ylidene]-6-[6-(4-methyl-imidazol-1-yl)-pyridin-3-yl]-imidazo[2,1-b]thiazol-3-one
2-[1-(3-Fluoro-4-hydroxy-5-propoxy-phenyl)-meth-(Z)-ylidene]-6-pyridin-4-yl-imidazo[2,1-b]thiazol-3-one
2-[1-(3-Chloro-5-(3-fluoropropoxy)-4-hydroxy-phenyl)-meth-(Z)-ylidene]-6-pyridin-4-yl-imidazo[2,1-b]thiazol-3-one
2-[1-(3-Fluoro-5-(3-fluoropropoxy)-4-hydroxy-phenyl)-meth-(Z)-ylidene]-6-pyridin-4-yl-imidazo[2,1-b]thiazol-3-one
2-[1-(3-Chloro-4-hydroxy-5-propoxy-phenyl)-meth-(Z)-ylidene]-6-pyridin-2-yl-imidazo[2,1-b]thiazol-3-one
2-[1-(3-Chloro-4-hydroxy-5-methoxy-phenyl)-meth-(Z)-ylidene]-6-(2-chloro-pyridin-4-yl)-imidazo[2,1-b]thiazol-3-one or
2-[1-[3-Chloro-5-(3-fluoro-propoxy)-4-hydroxy-phenyl]-meth-(Z)-ylidene]-6-(2,6-dimethyl-pyridin-4-yl)-imidazo[2,1-b]thiazol-3-one.

One further embodiment of the invention are compounds of formula IC

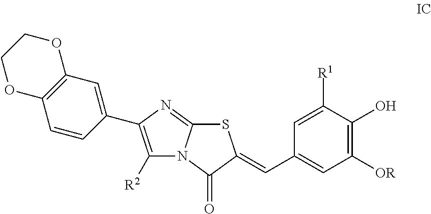

R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   or a pharmaceutically acceptable acid addition salt, a racemic mixture or its corresponding enantiomer and/or optical isomers thereof,
   for example
2-[1-(3-Chloro-4-hydroxy-5-methoxy-phenyl)-meth-(Z)-ylidene]-6-(2,3-dihydro-benzo[1,4]dioxin-6-yl)-imidazo[2,1-b]thiazol-3-one.

One further embodiment of the invention are compounds of formula ID,

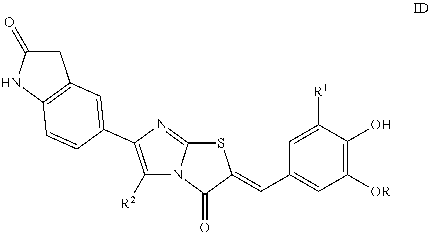

wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   and pharmaceutically acceptable salts thereof,
   for example
5-{2-[1-(3-Chloro-4-hydroxy-5-methoxy-phenyl)-meth-(Z)-ylidene]-3-oxo-2,3-dihydro-imidazo[2,1-b]thiazol-6-yl}-1,3-dihydro-indol-2-one.

One further embodiment of the invention are compounds of formula IE,

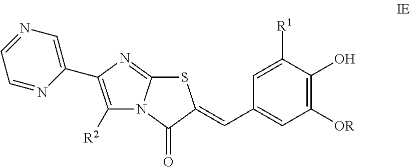

wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   or a pharmaceutically acceptable acid addition salt, a racemic mixture or its corresponding enantiomer and/or optical isomers thereof,
   for example
2-[1-(3-Chloro-4-hydroxy-5-propoxy-phenyl)-meth-(Z)-ylidene]-6-pyrazin-2-yl-imidazo[2,1-b]thiazol-3-one.

One further embodiment of the invention are compounds of formula IF

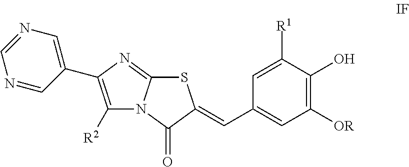

wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   or a pharmaceutically acceptable acid addition salt, a racemic mixture or its corresponding enantiomer and/or optical isomers thereof,
   for example
2-[1-(3-Chloro-4-hydroxy-5-propoxy-phenyl)-meth-(Z)-ylidene]-6-pyrimidin-5-yl-imidazo[2,1-b]thiazol-3-one.

One further embodiment of the invention are compounds of formula IG,

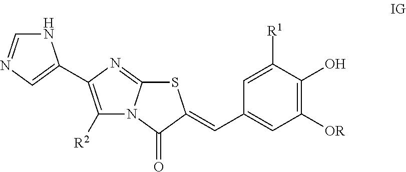

wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   or a pharmaceutically acceptable acid addition salt, a racemic mixture or its corresponding enantiomer and/or optical isomers thereof, for example
2-[1-(3-Chloro-4-hydroxy-5-propoxy-phenyl)-meth-(Z)-ylidene]-6-(3H-imidazol-4-yl)-imidazo[2,1-b]thiazol-3-one

One further embodiment of the invention are compounds of formula IH,

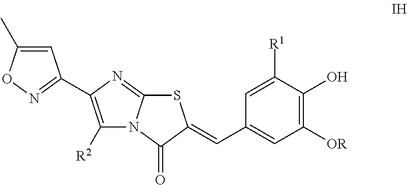

wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   or a pharmaceutically acceptable acid addition salt, a racemic mixture or its corresponding enantiomer and/or optical isomers thereof,
   for example
2-[1-(3-Chloro-4-hydroxy-5-methoxy-phenyl)-meth-(Z)-ylidene]-6-(5-methyl-isoxazol-3-yl)-imidazo[2,1-b]thiazol-3-one.

One further embodiment of the invention are compounds of formula IJ,

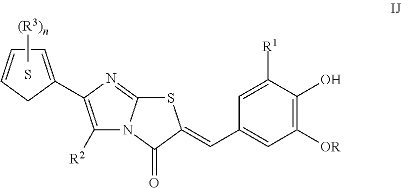

wherein
R³ is hydrogen or lower alkyl
n is 1 or 2
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
   or a pharmaceutically acceptable acid addition salt, a racemic mixture or its corresponding enantiomer and/or optical isomers thereof, for example
2-[1-(3-Chloro-4-hydroxy-5-propoxy-phenyl)-meth-(Z)-ylidene]-6-thiophen-3-yl-imidazo[2,1-b]thiazol-3-one
2-[1-(3-Chloro-4-hydroxy-5-propoxy-phenyl)-meth-(Z)-ylidene]-6-thiophen-2-yl-imidazo[2,1-b]thiazol-3-one or
2-[1-(3-Chloro-4-hydroxy-5-propoxy-phenyl)-meth-(Z)-ylidene]-6-(2,4-dimethylthiophen-3-yl)-imidazo[2,1-b]thiazol-3-one.

The compounds of formula I may be used in binding and imaging tau aggregates, beta-amyloid aggregates, alpha-synuclein aggregates or Huntington aggregates.

The preferred use of compounds of formula I is the use in binding and imaging tau aggregates in Alzheimer patients.

Furthermore, the compounds of formula I may be used in a tau-binding study.

The compounds of formula I are suitable for diagnostic imaging of tau-aggregates in the brain of a mammal.

In addition, the present invention comprises a pharmaceutical composition containing a compound of formula I and a pharmaceutical acceptable carrier.

Furthermore, the invention comprises a method of imaging tau-aggregate deposits, including
introducing into a mammal a detectable quantity of a composition;
allowing sufficient time for the compound of formula I to be associated with tau-aggregate deposit, and
detecting the compound associated with one or more tau-aggregate deposits.

One object of the invention is also the use of a compound of formula I for diagnostic imaging of tau-aggregate deposits in the brain of a mammal.

The present compounds of formulas

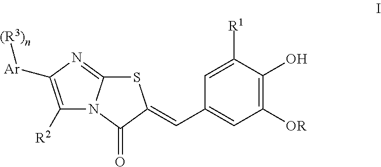

and their pharmaceutically acceptable salts can be prepared by processes described below, which process comprises
a) coupling a compound of formula Ia

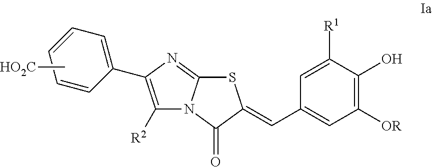

with an suitable amine HNR′R″ to afford a compound of formula I

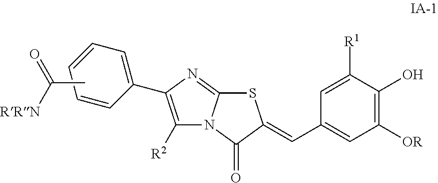

wherein the substituents R, R¹, R², R′ and R″ are as defined above,
and if desired, converting the compounds obtained into pharmaceutically acceptable acid addition salts;
or
b) coupling a compound with formula III

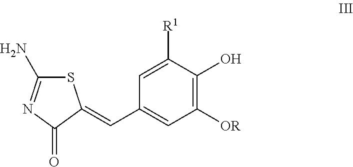

with a corresponding α-activated ketone of formula IVa

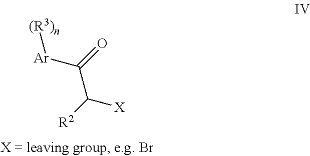

to afford a compound of formula I

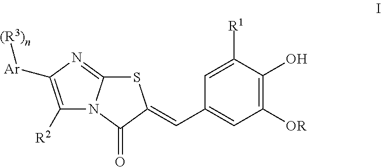

wherein the substituents R, R¹, R² and R³ are as defined above
and if desired, converting the compounds obtained into pharmaceutically acceptable acid addition salts.
or
c) coupling a compound with formula V

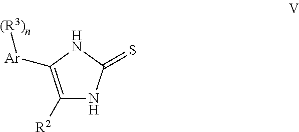

with a suitable acetic acid derivative and a corresponding aldehyde of formula VI

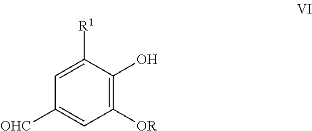

to afford a compound of formula I

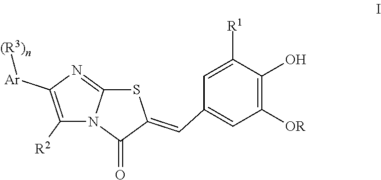

wherein the substituents R, R¹, R² and R³ are as defined above
and if desired, converting the compounds obtained into pharmaceutically acceptable acid addition salts.

The preparation of compounds of formula I of the present invention may be carried out in sequential or convergent synthetic routes. Syntheses of the compounds of the invention are shown in the following schemes 1 to 3. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. The reaction sequence is not limited to the one displayed in schemes 1 to 3, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

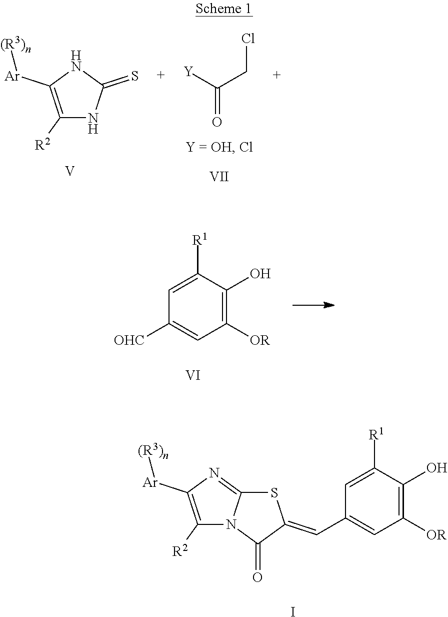

wherein the substituents R, R¹, R² and R³ are as defined above
According to scheme 1, derivatives of imidazothiazolone I are prepared via a condensation reaction of substituted 1,3-dihydro-imidazole-2-thiones V, an activated acetic acid derivative like chloroacetic acid or chloro-acetyl chloride in presence of a base, e.g. ethyldiisopropylamine or sodium acetate, and substituted benzaldehydes VI in a suitable solvent, e.g. acetic acid or dioxane, at elevated temperature.

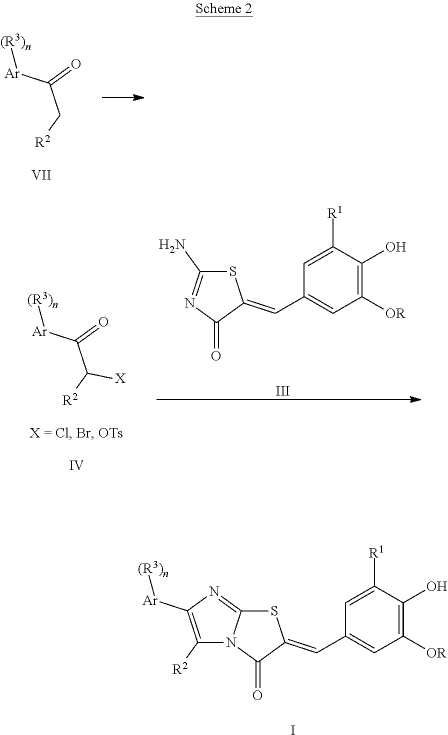

wherein the substituents R, R¹, R² and R³ are as defined above
According to scheme 2, an activated ketone IV with e.g. X═BR, is reacted with amino-thiazolone III in a suitable solvent, e.g. isopropanol, at elevated temperature in an oilbath or in a microwave to afford derivatives of compound I. An activated ketone IV can be synthesized in-situ by reacting ketone VII with an oxidation agent like [hydroxy(tosyloxy)iodo]benzene in a suitable solvent like acetonitrile affording the corresponding activated ketone IV with X═O-tosyl which can then react with aminothiazole III at elevated temperature yielding derivatives of imidazothiazolones I.

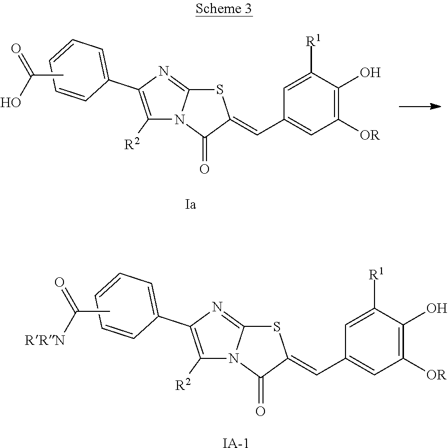

wherein the substituents R, R¹, R², R′ and R″ are as defined above
According to scheme 3, further derivatives of imidazothiazolones I are synthesized by coupling a corresponding carboxylic acid of formula Ia with a corresponding amine HNR′R″ by using a suitable amide bond coupling reagent, e.g. 2-(1H-benzo[d][1,2,3]triazol-1-yl)-1,1,3,3-tetramethylisouronium tetrafluoroborate, in a suitable solvent, e.g. N-methyl-2-pyrrolidinone, at ambient or elevated temperature.

### Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula IA or IB can be separated using chiral HPLC.

### Salts of Compounds of Formula I

The compounds of formula I are basic and may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

The acid addition salts of the basic compounds of formula I may be converted to the corresponding free bases by treatment with at least a stoichiometric equivalent of a suitable base such as sodium or potassium hydroxide, potassium carbonate, sodium bicarbonate, ammonia, and the like.

The compounds were investigated in accordance with the test given hereinafter.

### Assay for Determination of IC₁₅₀ for Tau and A-Beta

Recombinant human-microtubule associated protein Tau purified from E. coli is aggregated at a concentration of 5 μM with Arachidonic Acid (100 μM) in Tris 10 mM pH8, 24 h at 37° C. Synthetic Aβ40 is aggregated at a concentration of 50 μM with Arachidonic Acid (100 μM) in Tris 10 mM pH8, for three days at 37° C., under shaking at 150 rpm.

Human recombinant-Alpha-synuclein-purified from E. coli is aggregated at a concentration of 70 μM with Arachidonic Acid (100 μM) in Tris 10 mM pH 8, for 5 days at 37° C., under shaking at 150 rpm.

A saturation analysis of Thiazin-red R to the aggregated proteins is done to determine the affinity (Kd) of the Thiazin-red R to the aggregated protein. Table 1 shows the affinity constants of Thiazin-red R for aggregated tau, Abeta and alpha-synuclein. The results show that there are two binding sites with different affinity on each aggregated protein for Thiazin-red R.

Thiazin-red R will be added at the concentration corresponding to the Kd to the respective aggregated protein binding site, to induce a fluorescent signal that can be inhibited by the addition of a displacer compound.

To determine the affinity of a displacer compound to the Thiazin-red R binding sites of the aggregated proteins, the compound is added at different concentrations in the assay ranging from 0.3 nM to 10000 nM.

In parallel, auto fluorescence of the compound is measured together with the aggregated proteins, but without Thiazin-red R. As negative control, ligand and aggregated protein is used and as positive control, Thiazin-red R, reference compound with known activity and aggregated protein is used.

Assay is performed in Perkin Elmer OptiPlate 384, black, 45 ul assay volume, assay buffer is DPBS no CaCl₂ no MgCl₂ (GIBCO N. 14020). Tested compounds are diluted in DMSO and 2 μl is added to the assay (5% DMSO final). Assay is started by the addition of the aggregated protein (competitive condition). Plates are shortly shacked (1 min with Sterico variomag teleshake) and incubated for 30 min at room temperature. Measurement are done with En:Vision (Perkin Elmer), at Excitation 531 nm/Emission 595 nm.

TABLE

| | |
|---|---|
| | I |
| 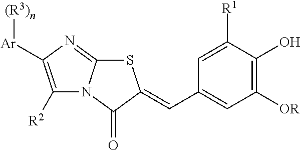 | |

| | | IC₅₀ | IC₅₀ | |
|---|---|---|---|---|
| Structure | Name | Tau | A-beta | Expl. |
| 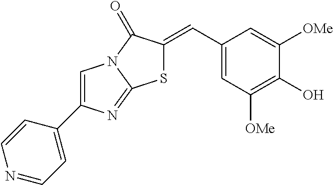 [SMILES: COc1cc(/C=c2\sc3nc(-c4ccncc4)cn3c2=O)cc(OC)c1O] | 2-[1-(4-Hydroxy-3,5- dimethoxy-phenyl)-meth- (Z)-ylidene]-6-pyridin-4- yl-imidazo[2,1-b]thiazol-3- one | 0.005 | 0.093 | 1 |
| 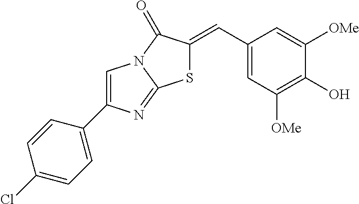 [SMILES: COc1cc(/C=c2\sc3nc(-c4ccc(Cl)cc4)cn3c2=O)cc(OC)c1O] | 6-(4-Chloro-phenyl)-2-[1- (4-hydroxy-3,5-dimethoxy- phenyl)-meth-(Z)-ylidene]- imidazo[2,1-b]thiazol-3- one | 0.002 | 0.007 | 2 |
| 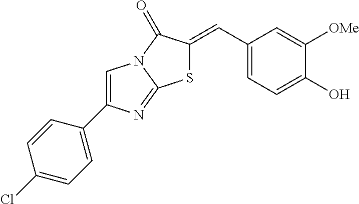 [SMILES: COc1cc(/C=c2\sc3nc(-c4ccc(Cl)cc4)cn3c2=O)ccc1O] | 6-(4-Chloro-phenyl)-2-[1- (4-hydroxy-3-methoxy- phenyl)-meth-(Z)-ylidene]- imidazo[2,1-b]thiazol-3- one | 0.049 | 0.215 | 3 |
| 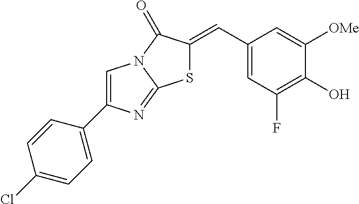 [SMILES: COc1cc(/C=c2\sc3nc(-c4ccc(Cl)cc4)cn3c2=O)cc(F)c1O] | 6-(4-Chloro-phenyl)-2-[1-(3- fluoro-4-hydroxy-5-methoxy- phenyl)-meth-(Z)-ylidene]- imidazo[2,1-b]thiazol-3-one | 0.011 | 0.393 | 4 |
| 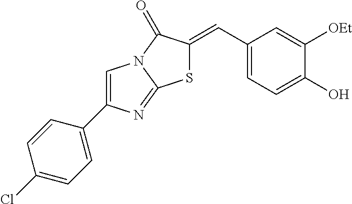 [SMILES: CCOc1cc(/C=c2\sc3nc(-c4ccc(Cl)cc4)cn3c2=O)ccc1O] | 6-(4-Chloro-phenyl)-2-[1- (3-ethoxy-4-hydroxy- phenyl)-meth-(Z)-ylidene]- imidazo[2,1-b]thiazol-3- one | 0.178 | 1.156 | 5 |
| 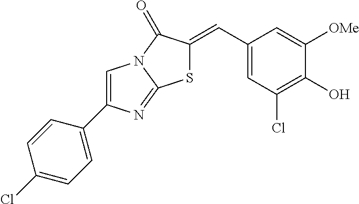 [SMILES: COc1cc(/C=c2\sc3nc(-c4ccc(Cl)cc4)cn3c2=O)cc(Cl)c1O] | 6-(4-Chloro-phenyl)-2-[1- (3-chloro-4-hydroxy-5- methoxy-phenyl)-meth-(Z)- ylidene]-imidazo[2,1- b]thiazol-3-one | 0.002 | 0.032 | 6 |
| 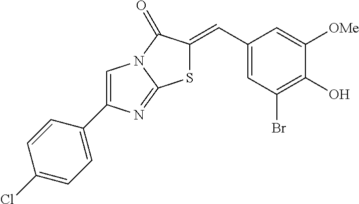 [SMILES: COc1cc(/C=c2\sc3nc(-c4ccc(Cl)cc4)cn3c2=O)cc(Br)c1O] | 6-(4-Chloro-phenyl)-2- [1-(3-bromo-4- hydroxy-5-methoxy- phenyl)-meth-(Z)- ylidene]-imidazo[2,1- b]thiazol-3-one | 0.002 | 0.018 | 7 |
| 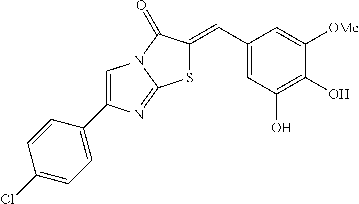 [SMILES: COc1cc(/C=c2\sc3nc(-c4ccc(Cl)cc4)cn3c2=O)cc(O)c1O] | 6-(4-Chloro-phenyl)-2-[1- (3,4-dihydroxy-5-methoxy- phenyl)-meth-(Z)-ylidene]- imidazo[2,1-b]thiazol-3- one | 0.005 | 0.018 | 8 |
| 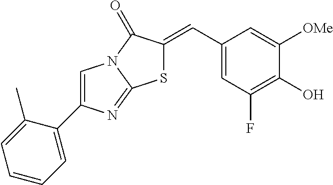 [SMILES: COc1cc(/C=c2\sc3nc(-c4ccccc4C)cn3c2=O)cc(F)c1O] | 2-[1-(3-Fluoro-4-hydroxy- 5-methoxy-phenyl)-meth- (Z)-ylidene]-6-o-tolyl- imidazo[2,1-b]thiazol-3- one | 0.105 | 0.706 | 9 |
| 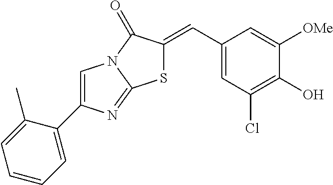 [SMILES: COc1cc(/C=c2\sc3nc(-c4ccccc4C)cn3c2=O)cc(Cl)c1O] | 2-[1-(3-Chloro-4-hydroxy- 5-methoxy-phenyl)-meth- (Z)-ylidene]-6-o-tolyl- imidazo[2,1-b]thiazol-3- one | 0.028 | 1.217 | 10 |
| 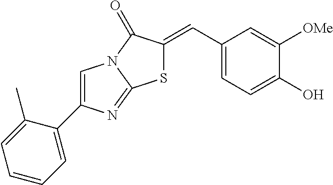 [SMILES: COc1cc(/C=c2\sc3nc(-c4ccccc4C)cn3c2=O)ccc1O] | 2-[1-(4-hydroxy-3- methoxy-phenyl)-meth-(Z)- ylidene]-6-o-tolyl- imidazo[2,1-b]thiazol-3- one | 0.837 | 1.283 | 11 |
| 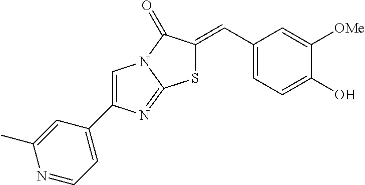 [SMILES: COc1cc(/C=c2\sc3nc(-c4ccnc(C)c4)cn3c2=O)ccc1O] | 2-[1-(4-Hydroxy-3- methoxy-phenyl)-meth-(Z)- ylidene]-6-(2-methyl- pyridin-4-yl)-imidazo[2,1- b]thiazol-3-one | 0.079 | >10 | 12 |
| 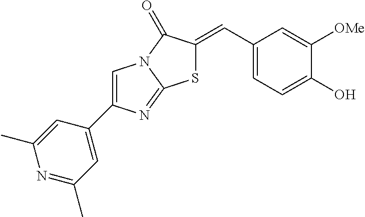 [SMILES: COc1cc(/C=c2\sc3nc(-c4cc(C)nc(C)c4)cn3c2=O)ccc1O] | 2-[1-(4-Hydroxy-3- methoxy-phenyl)-meth-(Z)- ylidene]-6-(2,6-dimethyl- pyridin-4-yl)-imidazo[2,1- b]thiazol-3-one | 0.091 | >10 | 13 |
| 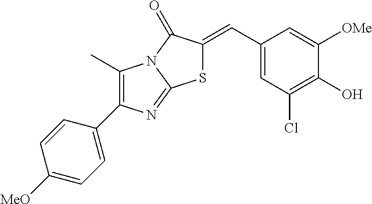 [SMILES: COc1ccc(-c2nc3s/c(=C\c4cc(Cl)c(O)c(OC)c4)c(=O)n3c2C)cc1] | 2-[1-(3-Chloro-4-hydroxy- 5-methoxy-phenyl)-meth- (Z)-ylidene]-6-(4-methoxy- phenyl)-5-methyl- imidazo[2,1-b]thiazol-3- one | 0.037 | 0.466 | 14 |
| 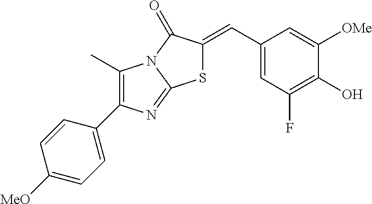 [SMILES: COc1ccc(-c2nc3s/c(=C\c4cc(F)c(O)c(OC)c4)c(=O)n3c2C)cc1] | 2-[1-(3-Fluoro-4-hydroxy- 5-methoxy-phenyl)-meth- (Z)-ylidene]-6-(4-methoxy- phenyl)-5-methyl- imidazo[2,1-b]thiazol-3- one | 0.053 | 0.437 | 15 |
| 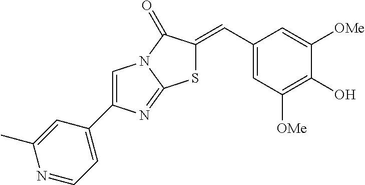 [SMILES: COc1cc(/C=c2\sc3nc(-c4ccnc(C)c4)cn3c2=O)cc(OC)c1O] | 2-[1-(4-Hydroxy-3,5- dimethoxy-phenyl)-meth- (Z)-ylidene]-6-(2-methyl- pyridin-4-yl)-imidazo[2,1- b]thiazol-3-one | 0.008 | 0.032 | 16 |
| 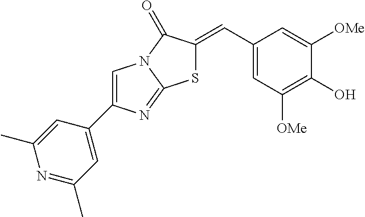 [SMILES: COc1cc(/C=c2\sc3nc(-c4cc(C)nc(C)c4)cn3c2=O)cc(OC)c1O] | 2-[1-(4-Hydroxy-3,5- dimethoxy-phenyl)-meth- (Z)-ylidene]-6-(2,6- dimethyl-pyridin-4-yl)- imidazo[2,1-b]thiazol-3- one | 0.005 | 0.068 | 17 |
| 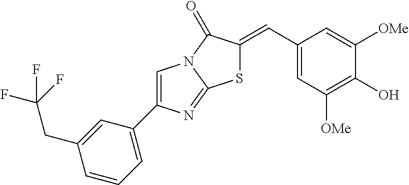 [SMILES: COc1cc(/C=c2\sc3nc(-c4cccc(CC(F)(F)F)c4)cn3c2=O)cc(OC)c1O] | 2-[1-(4-Hydroxy-3,5- dimethoxy-phenyl)-meth- (Z)-ylidene]-6-(3- trifluoromethoxy-phenyl)- imidazo[2,1-b]thiazol-3- one | 0.004 | 0.114 | 18 |
| 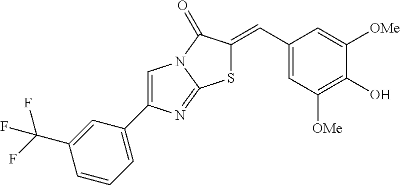 [SMILES: COc1cc(/C=c2\sc3nc(-c4cccc(C(F)(F)F)c4)cn3c2=O)cc(OC)c1O] | 2-[1-(4-Hydroxy-3,5- dimethoxy-phenyl)-meth- (Z)-ylidene]-6-(3- trifluoromethyl-phenyl)- imidazo[2,1-b]thiazol-3- one | 0.001 | 0.115 | 19 |
| 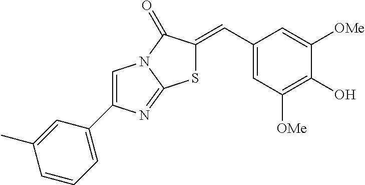 [SMILES: COc1cc(/C=c2\sc3nc(-c4cccc(C)c4)cn3c2=O)cc(OC)c1O] | 2-[1-(4-Hydroxy-3,5- dimethoxy-phenyl)-meth- (Z)-ylidene]-6-m-tolyl- imidazo[2,1-b]thiazol-3- one | 0.003 | 0.020 | 20 |
| 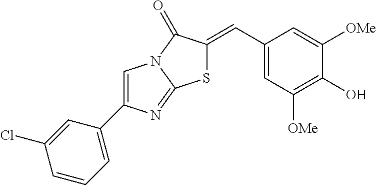 [SMILES: COc1cc(/C=c2\sc3nc(-c4cccc(Cl)c4)cn3c2=O)cc(OC)c1O] | 6-(3-Chloro-phenyl)-2-[1- (4-hydroxy-3,5-dimethoxy- phenyl)-meth-(Z)-ylidene]- imidazo[2,1-b]thiazol-3- one | 0.001 | 0.006 | 21 |
| 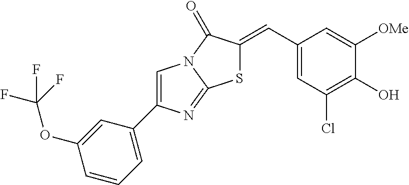 [SMILES: COc1cc(/C=c2\sc3nc(-c4cccc(OC(F)(F)F)c4)cn3c2=O)cc(Cl)c1O] | 2-[1-(3-Chloro-4-hydroxy- 5-methoxy-phenyl)-meth- (Z)-ylidene]-6-(3- trifluoromethoxy-phenyl)- imidazo[2,1-b]thiazol-3- one | 0.003 | 0.198 | 22 |
| 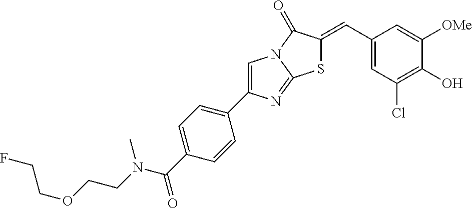 [SMILES: COc1cc(/C=c2\sc3nc(-c4ccc(C(=O)N(C)CCOCCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-[2-(2-fluoro-ethoxy)- ethyl]-N-methyl-benzamide | 0.026 | 0.328 | 23 |
| 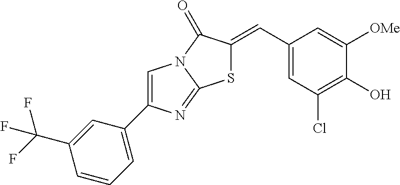 [SMILES: COc1cc(/C=c2\sc3nc(-c4cccc(C(F)(F)F)c4)cn3c2=O)cc(Cl)c1O] | 2-[1-(3-Chloro-4-hydroxy- 5-methoxy-phenyl)-meth- (Z)-ylidene]-6-(3- trifluoromethyl-phenyl)- imidazo[2,1-b]thiazol-3- one | 0.003 | 0.123 | 24 |
| 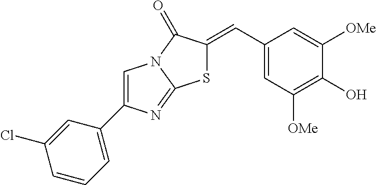 [SMILES: COc1cc(/C=c2\sc3nc(-c4cccc(Cl)c4)cn3c2=O)cc(OC)c1O] | 6-(3-Chloro-phenyl)-2-[1- (3-chloro-4-hydroxy-5- methoxy-phenyl)-meth-(Z)- ylidene]-imidazo[2,1- b]thiazol-3-one | 0.0005 | 0.253 | 25 |
| 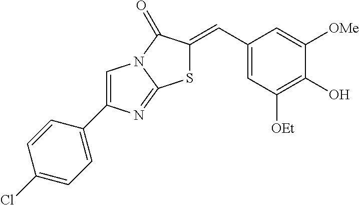 [SMILES: CCOc1cc(/C=c2\sc3nc(-c4ccc(Cl)cc4)cn3c2=O)cc(OC)c1O] | 6-(4-Chloro-phenyl)-2-[1- (3-ethoxy-4-hydroxy-5- methoxy-phenyl)-meth-(Z)- ylidene]-imidazo[2,1- b]thiazol-3-one | 0.002 | 0.004 | 26 |
| 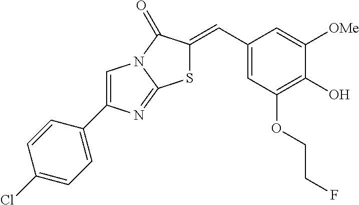 [SMILES: COc1cc(/C=c2\sc3nc(-c4ccc(Cl)cc4)cn3c2=O)cc(OCCF)c1O] | 6-(4-Chloro-phenyl)-2-[1- (3-(2-fluoro-ethoxy)-4- hydroxy-5-methoxy- phenyl)-meth-(Z)-ylidene]- imidazo[2,1-b]thiazol-3- one | 0.001 | 0.007 | 27 |
| 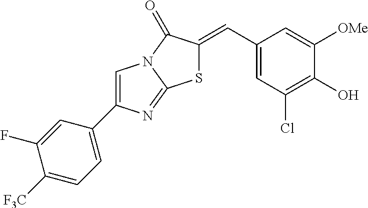 [SMILES: COc1cc(/C=c2\sc3nc(-c4ccc(C(F)(F)F)c(F)c4)cn3c2=O)cc(Cl)c1O] | 2-[1-(3-Chloro-4-hydroxy- 5-methoxy-phenyl)-meth- (Z)-ylidene]-6-(3-fluoro-4- trifluoromethyl-phenyl)- imidazo[2,1-b]thiazol-3- one | 0.023 | 1.056 | 28 |
| 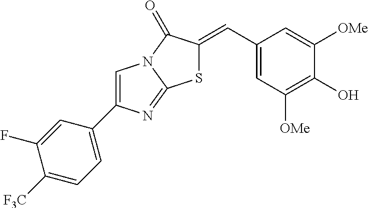 [SMILES: COc1cc(/C=c2\sc3nc(-c4ccc(C(F)(F)F)c(F)c4)cn3c2=O)cc(OC)c1O] | 2-[1-(3-Hydroxy-3,5- dimethoxy-phenyl)-meth- (Z)-ylidene]-6-(3-fluoro-4- trifluoromethyl-phenyl)- imidazo[2,1-b]thiazol-3- one | 0.011 | 0.043 | 29 |
| 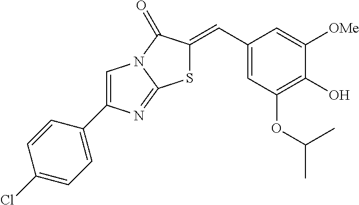 [SMILES: COc1cc(/C=c2\sc3nc(-c4ccc(Cl)cc4)cn3c2=O)cc(OC(C)C)c1O] | 6-(4-Chloro-phenyl)-2-[1- (4-hydroxy-3-isopropoxy- 5-methoxy-phenyl)-meth- (Z)-ylidene]-imidazo[2,1- b]thiazol-3-one | 0.006 | 0.016 | 30 |
| 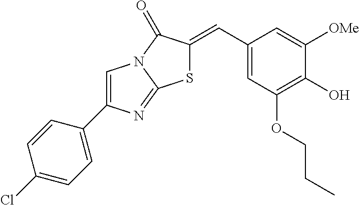 [SMILES: CCCOc1cc(/C=c2\sc3nc(-c4ccc(Cl)cc4)cn3c2=O)cc(OC)c1O] | 6-(4-Chloro-phenyl)-2-[1- (4-hydroxy-3-methoxy-5- propoxy-phenyl)-meth-(Z)- ylidene]-imidazo[2,1- b]thiazol-3-one | 0.003 | 0.012 | 31 |
| 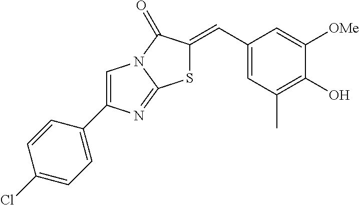 [SMILES: COc1cc(/C=c2\sc3nc(-c4ccc(Cl)cc4)cn3c2=O)cc(C)c1O] | 6-(4-Chloro-phenyl)-2-[1- (4-hydroxy-3-methoxy-5- methyl-phenyl)-meth-(Z)- ylidene]-imidazo[2,1- b]thiazol-3-one | 0.007 | 0.039 | 32 |
| 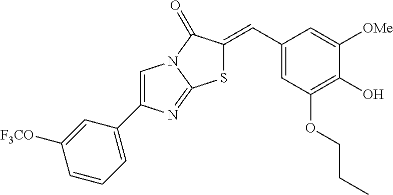 [SMILES: CCCOc1cc(/C=c2\sc3nc(-c4cccc(OC(F)(F)F)c4)cn3c2=O)cc(OC)c1O] | 2-[1-(4-Hydroxy-3- methoxy-5-propoxy- phenyl)-meth-(Z)-ylidene]- 6-(3-trifluoromethoxy- phenyl)-imidazo[2,1- b]thiazol-3-one | 0.025 | 0.256 | 33 |
| 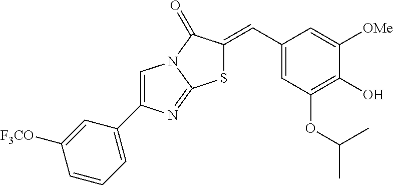 [SMILES: COc1cc(/C=c2\sc3nc(-c4cccc(OC(F)(F)F)c4)cn3c2=O)cc(OC(C)C)c1O] | 2-[1-(4-Hydroxy-3- methoxy-5-isopropoxy- phenyl)-meth-(Z)-ylidene]- 6-(3-trifluoromethoxy- phenyl)-imidazo[2,1- b]thiazol-3-one | 0.026 | 0.437 | 34 |
| 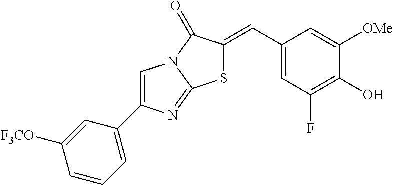 [SMILES: COc1cc(/C=c2\sc3nc(-c4cccc(OC(F)(F)F)c4)cn3c2=O)cc(F)c1O] | 2-[1-(3-Fluoro-4-hydroxy- 5-methoxy-phenyl)-meth- (Z)-ylidene]-6-(3- trifluoromethoxy-phenyl)- imidazo[2,1-b]thiazol-3- one | 0.013 | >10 | 35 |
| 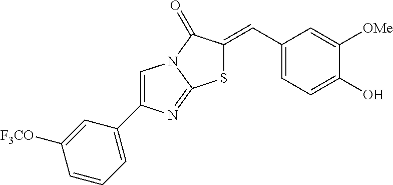 [SMILES: COc1cc(/C=c2\sc3nc(-c4cccc(OC(F)(F)F)c4)cn3c2=O)ccc1O] | 2-[1-(4-Hydroxy-3- methoxy-phenyl)-meth-(Z)- ylidene]-6-(3- trifluoromethoxy-phenyl)- imidazo[2,1-b]thiazol-3- one | 0.401 | >10 | 36 |
| 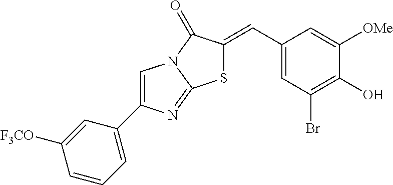 [SMILES: COc1cc(/C=c2\sc3nc(-c4cccc(OC(F)(F)F)c4)cn3c2=O)cc(Br)c1O] | 2-[1-(3-Bromo-4-hydroxy- 5-methoxy-phenyl)-meth- (Z)-ylidene]-6-(3- trifluoromethoxy-phenyl)- imidazo[2,1-b]thiazol-3- one | 0.006 | 0.552 | 37 |
| 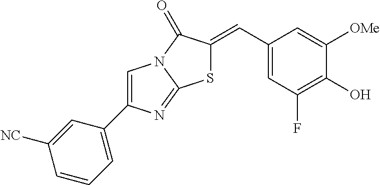 [SMILES: COc1cc(/C=c2\sc3nc(-c4cccc(C#N)c4)cn3c2=O)cc(F)c1O] | 3-{2-[1-(3-Fluoro-4- hydroxy-5-methoxy- phenyl)-meth-(Z)-ylidene]- 3-oxo-2,3-dihydro- imidazo[2,1-b]thiazol-6- yl}-benzonitrile | 0.021 | >10 | 38 |
| 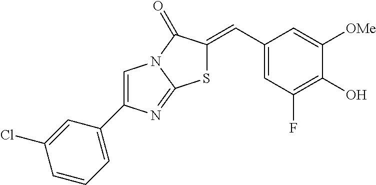 [SMILES: COc1cc(/C=c2\sc3nc(-c4cccc(Cl)c4)cn3c2=O)cc(F)c1O] | 2-[1-(3-Fluoro-4-hydroxy- 5-methoxy-phenyl)-meth- (Z)-ylidene]-6-(3-chloro- phenyl)-imidazo[2,1- b]thiazol-3-one | 0.011 | 0.412 | 39 |
| 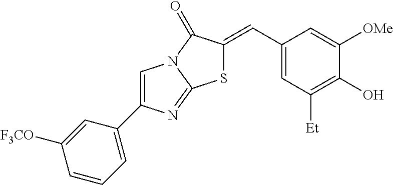 [SMILES: CCc1cc(/C=c2\sc3nc(-c4cccc(OC(F)(F)F)c4)cn3c2=O)cc(OC)c1O] | 2-[1-(3-Ethyl-4-hydroxy-5- methoxy-phenyl)-meth-(Z)- ylidene]-6-(3- trifluoromethoxy-phenyl)- imidazo[2,1-b]thiazol-3- one | 0.701 | >10 | 40 |
| 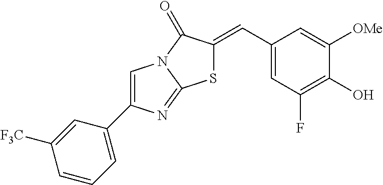 [SMILES: COc1cc(/C=c2\sc3nc(-c4cccc(C(F)(F)F)c4)cn3c2=O)cc(F)c1O] | 2-[1-(3-fluoro-4-hydroxy- 5-methoxy-phenyl)-meth- (Z)-ylidene]-6-(3- (trifluoromethyl)-phenyl)- imidazo[2,1-b]thiazol-3- one | 0.016 | 1.810 | 41 |
| 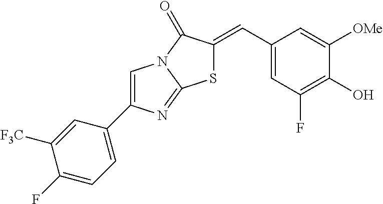 [SMILES: COc1cc(/C=c2\sc3nc(-c4ccc(F)c(C(F)(F)F)c4)cn3c2=O)cc(F)c1O] | 2-[1-(3-fluoro-4-hydroxy- 5-methoxy-phenyl)-meth- (Z)-ylidene]-6-(4-fluoro-3- (trifluoromethyl)-phenyl)- imidazo[2,1-b]thiazol-3- one | 0.024 | 0.198 | 42 |
| 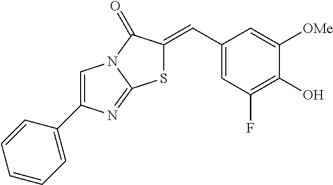 [SMILES: COc1cc(/C=c2\sc3nc(-c4ccccc4)cn3c2=O)cc(F)c1O] | 2-[1-(3-fluoro-4-hydroxy- 5-methoxy-phenyl)-meth- (Z)-ylidene]-6-phenyl- imidazo[2,1-b]thiazol-3- one | 0.051 | 2.320 | 43 |
| 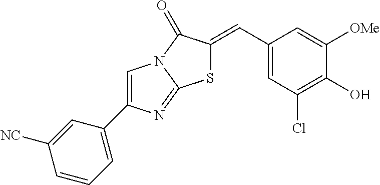 [SMILES: COc1cc(/C=c2\sc3nc(-c4cccc(C#N)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzonitrile | 0.006 | 24.015 | 44 |
| 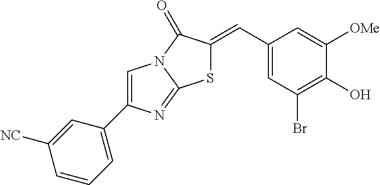 [SMILES: COc1cc(/C=c2\sc3nc(-c4cccc(C#N)c4)cn3c2=O)cc(Br)c1O] | 3-{2-[1-(3-Bromo-4- hydroxy-5-methoxy- phenyl)-meth-(Z)-ylidene]- 3-oxo-2,3-dihydro- imidazo[2,1-b]thiazol-6- yl}-benzonitrile | 0.025 | >10 | 45 |
| 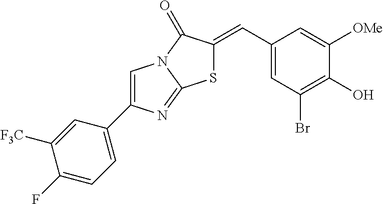 [SMILES: COc1cc(/C=c2\sc3nc(-c4ccc(F)c(C(F)(F)F)c4)cn3c2=O)cc(Br)c1O] | 2-[1-(3-bromo-4-hydroxy- 5-methoxy-phenyl)-meth- (Z)-ylidene]-6-(4-fluoro-3- (trifluoromethyl)-phenyl)- imidazo[2,1-b]thiazol-3- one | 0.028 | 5.9105 | 46 |
| 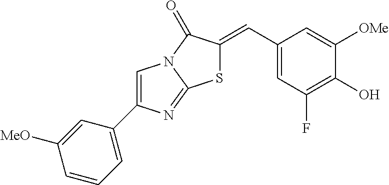 [SMILES: COc1cccc(-c2cn3c(=O)/c(=C/c4cc(F)c(O)c(OC)c4)sc3n2)c1] | 2-[1-(3-Fluoro-4-hydroxy- 5-methoxy-phenyl)-meth- (Z)-ylidene]-6-(3-methoxy- phenyl)-imidazo[2,1- b]thiazol-3-one | 0.017 | 0.540 | 47 |
| 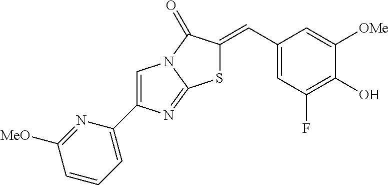 [SMILES: COc1cccc(-c2cn3c(=O)/c(=C/c4cc(F)c(O)c(OC)c4)sc3n2)n1] | 2-[1-(3-Fluoro-4-hydroxy- 5-methoxy-phenyl)-meth- (Z)-ylidene]-6-(6-methoxy- pyridin-2-yl)-imidazo[2,1- b]thiazol-3-one | 0.020 | 1.790 | 48 |
| 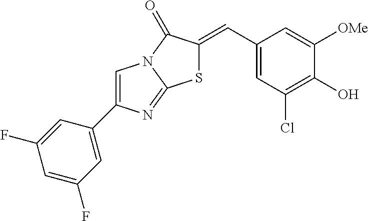 [SMILES: COc1cc(/C=c2\sc3nc(-c4cc(F)cc(F)c4)cn3c2=O)cc(Cl)c1O] | 2-[1-(3-Chloro-4-hydroxy- 5-methoxy-phenyl)-meth- (Z)-ylidene]-6-(3,5- difluoro-phenyl)- imidazo[2,1-b]thiazol-3- one | 0.016 | 1.371 | 49 |
| 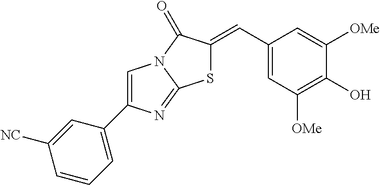 [SMILES: COc1cc(/C=c2\sc3nc(-c4cccc(C#N)c4)cn3c2=O)cc(OC)c1O] | 3-{2-[1-(4-Hydroxy-3,5- dimethoxy-phenyl)-meth- (Z)-ylidene]-3-oxo-2,3- dihydro-imidazo[2,1- b]thiazol-6-yl}-benzonitrile | 0.007 | | 50 |
| 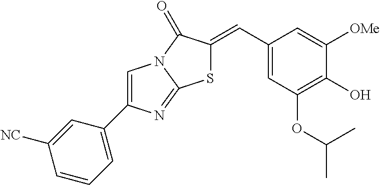 [SMILES: COc1cc(/C=c2\sc3nc(-c4cccc(C#N)c4)cn3c2=O)cc(OC(C)C)c1O] | 3-{2-[1-(4-Hydroxy-3- methoxy-5- isopropoxyphenyl)-meth- (Z)-ylidene]-3-oxo-2,3- dihydro-imidazo[2,1- b]thiazol-6-yl}-benzonitrile | 0.275 | >10 | 51 |
| 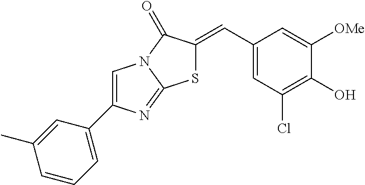 [SMILES: COc1cc(/C=c2\sc3nc(-c4cccc(C)c4)cn3c2=O)cc(Cl)c1O] | 2-[1-(3-Chloro-4-hydroxy- 5-methoxy-phenyl)-meth- (Z)-ylidene]-6-m-tolyl- imidazo[2,1-b]thiazol-3- one | 0.007 | 1.023 | 52 |
| 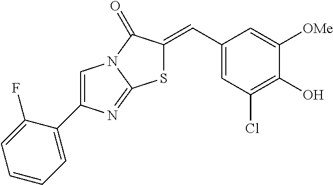 [SMILES: COc1cc(/C=c2\sc3nc(-c4ccccc4F)cn3c2=O)cc(Cl)c1O] | 2-[1-(3-Chloro-4-hydroxy- 5-methoxy-phenyl)-meth- (Z)-ylidene]-6-(2-fluoro- phenyl)-imidazo[2,1- b]thiazol-3-one | 0.016 | 5.649 | 53 |
| 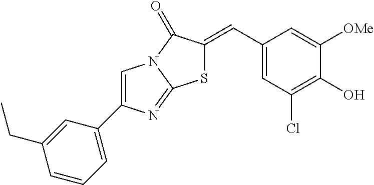 [SMILES: CCc1cccc(-c2cn3c(=O)/c(=C/c4cc(Cl)c(O)c(OC)c4)sc3n2)c1] | 2-[1-(3-Chloro-4-hydroxy- 5-methoxy-phenyl)-meth- (Z)-ylidene]-6-(3-ethyl- phenyl)-imidazo[2,1- b]thiazol-3-one | 0.025 | 0.653 | 54 |
| 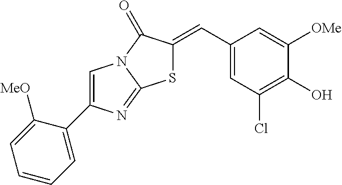 [SMILES: COc1ccccc1-c1cn2c(=O)/c(=C/c3cc(Cl)c(O)c(OC)c3)sc2n1] | 2-[1-(3-Chloro-4-hydroxy- 5-methoxy-phenyl)-meth- (Z)-ylidene]-6-(2-methoxy- phenyl)-imidazo[2,1- b]thiazol-3-one | 0.103 | | 55 |
| 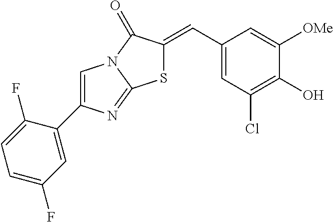 [SMILES: COc1cc(/C=c2\sc3nc(-c4cc(F)ccc4F)cn3c2=O)cc(Cl)c1O] | 2-[1-(3-Chloro-4-hydroxy- 5-methoxy-phenyl)-meth- (Z)-ylidene]-6-(2,5- difluoro-phenyl)- imidazo[2,1-b]thiazol-3- one | 0.027 | | 56 |
| 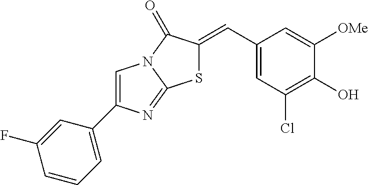 [SMILES: COc1cc(/C=c2\sc3nc(-c4cccc(F)c4)cn3c2=O)cc(Cl)c1O] | 2-[1-(3-Chloro-4-hydroxy- 5-methoxy-phenyl)-meth- (Z)-ylidene]-6-(3-fluoro- phenyl)-imidazo[2,1- b]thiazol-3-one | 0.006 | 3.323 | 57 |
| 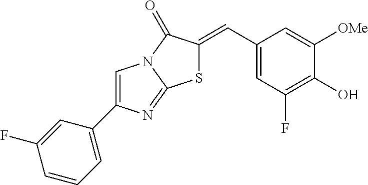 [SMILES: COc1cc(/C=c2\sc3nc(-c4cccc(F)c4)cn3c2=O)cc(F)c1O] | 2-[1-(3-Fluoro-4-hydroxy- 5-methoxy-phenyl)-meth- (Z)-ylidene]-6-(3-fluoro- phenyl)-imidazo[2,1- b]thiazol-3-one | 0.019 | 0.752 | 58 |
| 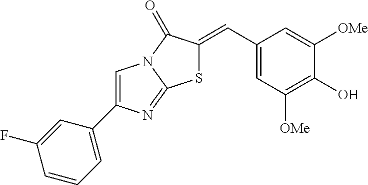 [SMILES: COc1cc(/C=c2\sc3nc(-c4cccc(F)c4)cn3c2=O)cc(OC)c1O] | 2-[1-(4-Hydroxy-3,5- dimethoxy-phenyl)-meth- (Z)-ylidene]-6-(3-fluoro- phenyl)-imidazo[2,1- b]thiazol-3-one | 0.002 | 0.05 | 59 |
| 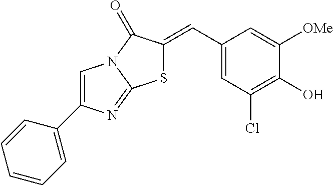 [SMILES: COc1cc(/C=c2\sc3nc(-c4ccccc4)cn3c2=O)cc(Cl)c1O] | 2-[1-(3-Chloro-4-hydroxy- 5-methoxy-phenyl)-meth- (Z)-ylidene]-6-phenyl- imidazo[2,1-b]thiazol-3- one | 0.025 | 0.851 | 60 |
| 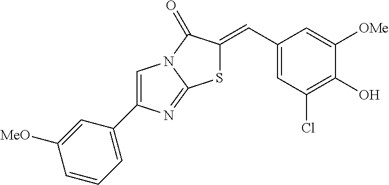 [SMILES: COc1cccc(-c2cn3c(=O)/c(=C/c4cc(Cl)c(O)c(OC)c4)sc3n2)c1] | 2-[1-(3-Chloro-4-hydroxy- 5-methoxy-phenyl)-meth- (Z)-ylidene]-6-(3-methoxy- phenyl)-imidazo[2,1- b]thiazol-3-one | 0.004 | 0.395 | 61 |
| 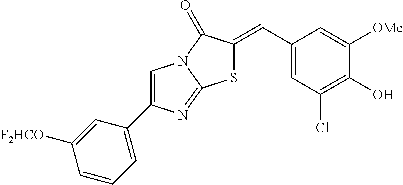 [SMILES: COc1cc(/C=c2\sc3nc(-c4cccc(OC(F)F)c4)cn3c2=O)cc(Cl)c1O] | 2-[1-(3-Chloro-4-hydroxy- 5-methoxy-phenyl)-meth- (Z)-ylidene]-6-(3- (difluoromethoxy)-phenyl)- imidazo[2,1-b]thiazol-3- one | 0.006 | 4.161 | 62 |
| 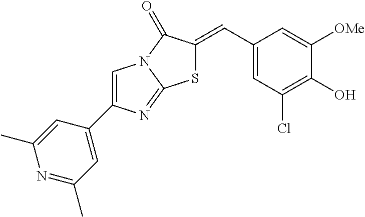 [SMILES: COc1cc(/C=c2\sc3nc(-c4cc(C)nc(C)c4)cn3c2=O)cc(Cl)c1O] | 2-[1-(3-Chloro-4-hydroxy- 5-methoxy-phenyl)-meth- (Z)-ylidene]-6-(2,6- dimethyl-pyridin-4-yl)- imidazo[2,1-b]thiazol-3- one | 0.013 | 0.418 | 63 |
| 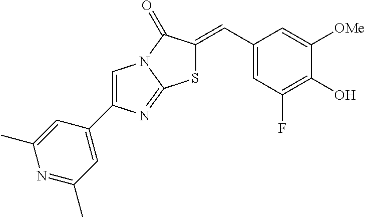 [SMILES: COc1cc(/C=c2\sc3nc(-c4cc(C)nc(C)c4)cn3c2=O)cc(F)c1O] | 2-[1-(3-Fluoro-4-hydroxy- 5-methoxy-phenyl)-meth- (Z)-ylidene]-6-(2,6- dimethyl-pyridin-4-yl)- imidazo[2,1-b]thiazol-3- one | 0.032 | 0.406 | 64 |
| 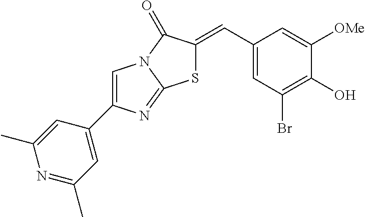 [SMILES: COc1cc(/C=c2\sc3nc(-c4cc(C)nc(C)c4)cn3c2=O)cc(Br)c1O] | 2-[1-(3-Bromo-4-hydroxy- 5-methoxy-phenyl)-meth- (Z)-ylidene]-6-(2,6- dimethyl-pyridin-4-yl)- imidazo[2,1-b]thiazol-3- one | 0.007 | 0.304 | 65 |
| 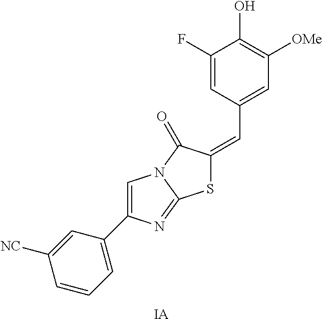 [SMILES: COc1cc(/C=c2/sc3nc(-c4cccc(C#N)c4)cn3c2=O)cc(F)c1O ; "IA"] | (Z)-3-(2-(3-fluoro-4- hydroxy-5- methoxybenzylidene)-3- oxo-2,3- dihydroimidazo[2,1-b] thiazol-5-yl)benzonitrile | 0.004 | 0.008 | 66 |
| 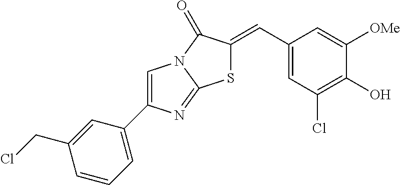 [SMILES: COc1cc(/C=c2\sc3nc(-c4cccc(CCl)c4)cn3c2=O)cc(Cl)c1O] | 2-[1-(3-Chloro-4-hydroxy- 5-methoxy-phenyl)-meth- (Z)-ylidene]-6-(3- (chloromethyl)phenyl)- imidazo[2,1-b]thiazol-3- one | 0.0008 | 0.009 | 67 |
| 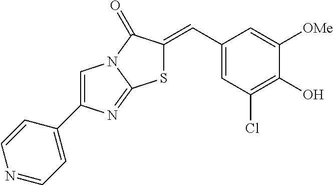 [SMILES: COc1cc(/C=c2\sc3nc(-c4ccncc4)cn3c2=O)cc(Cl)c1O] | 2-[1-(3-Chloro-4-hydroxy- 5-methoxy-phenyl)-meth- (Z)-ylidene]-6-(pyridin-4- yl)-imidazo[2,1-b]thiazol- 3-one | 0.003 | 0.300 | 68 |
| 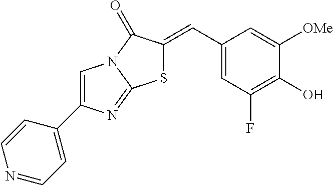 [SMILES: COc1cc(/C=c2\sc3nc(-c4ccncc4)cn3c2=O)cc(F)c1O] | 2-[1-(3-Fluoro-4-hydroxy- 5-methoxy-phenyl)-meth- (Z)-ylidene]-6-(pyridin-4- yl)-imidazo[2,1-b]thiazol- 3-one | 0.020 | 0.62 | 69 |
| 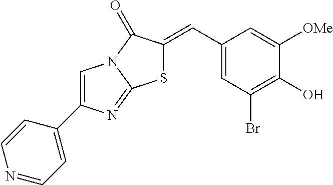 [SMILES: COc1cc(/C=c2\sc3nc(-c4ccncc4)cn3c2=O)cc(Br)c1O] | 2-[1-(3-Bromo-4-hydroxy- 5-methoxy-phenyl)-meth- (Z)-ylidene]-6-(pyridin-3- yl)-imidazo[2,1-b]thiazol- 3-one | 0.0013 | 0.103 | 70 |
| 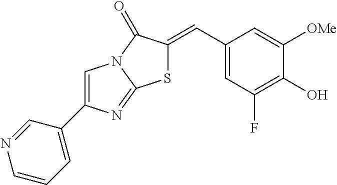 [SMILES: COc1cc(/C=c2\sc3nc(-c4cccnc4)cn3c2=O)cc(F)c1O] | 2-[1-(3-Fluoro-4-hydroxy- 5-methoxy-phenyl)-meth- (Z)-ylidene]-6-(pyridin-3- yl)-imidazo[2,1-b]thiazol- 3-one | 0.020 | 0.238 | 71 |
| 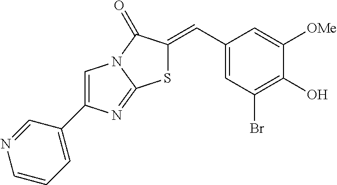 [SMILES: COc1cc(/C=c2\sc3nc(-c4cccnc4)cn3c2=O)cc(Br)c1O] | 2-[1-(3-Bromo-4-hydroxy- 5-methoxy-phenyl)-meth- (Z)-ylidene]-6-(pyridin-3- yl)-imidazo[2,1-b]thiazol- 3-one | 0.003 | 0.229 | 72 |
| 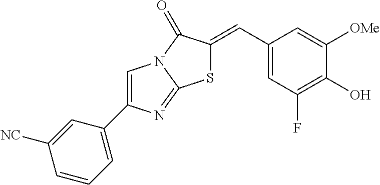 [SMILES: COc1cc(/C=c2\sc3nc(-c4cccc(C#N)c4)cn3c2=O)cc(F)c1O] | 3-{2-[1-(3-Fluoro-4- hydroxy-5-methoxy- phenyl)-meth-(Z)-ylidene]- 3-oxo-2,3-dihydro- imidazo[2,1-b]thiazol-6- yl}-benzonitrile | 0.003 | 0.013 | 73 |
| 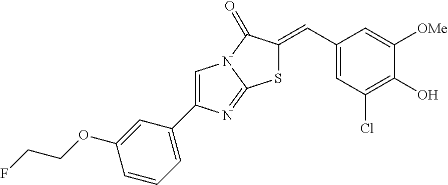 [SMILES: COc1cc(/C=c2\sc3nc(-c4cccc(OCCF)c4)cn3c2=O)cc(Cl)c1O] | 2-[1-(3-Chloro-4-hydroxy- 5-methoxy-phenyl)-meth- (Z)-ylidene]-6-(3-(2- fluoroethoxy)phenyl)- imidazo[2,1-b]thiazol-3- one | 0.005 | 0.055 | 74 |
| 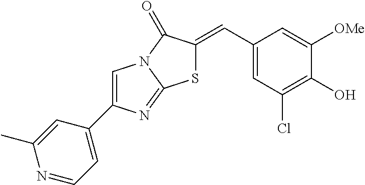 [SMILES: COc1cc(/C=c2\sc3nc(-c4ccnc(C)c4)cn3c2=O)cc(Cl)c1O] | 2-[1-(3-Chloro-4-hydroxy- 5-methoxy-phenyl)-meth- (Z)-ylidene]-6-(2- methylpyridin-4-yl)- imidazo[2,1-b]thiazol-3- one | 0.0013 | 0.122 | 75 |
| 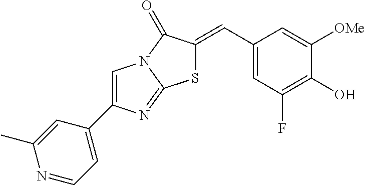 [SMILES: COc1cc(/C=c2\sc3nc(-c4ccnc(C)c4)cn3c2=O)cc(F)c1O] | 2-[1-(3-Fluoro-4-hydroxy- 5-methoxy-phenyl)-meth- (Z)-ylidene]-6-(2- methylpyridin-4-yl)- imidazo[2,1-b]thiazol-3- one | 0.006 | 0.260 | 76 |
| 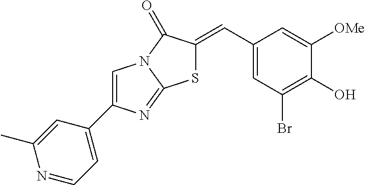 [SMILES: COc1cc(/C=c2\sc3nc(-c4ccnc(C)c4)cn3c2=O)cc(Br)c1O] | 2-[1-(3-Bromo-4-hydroxy- 5-methoxy-phenyl)-meth- (Z)-ylidene]-6-(2- methylpyridin-4-yl)- imidazo[2,1-b]thiazol-3- one | 0.001 | 0.076 | 77 |
| 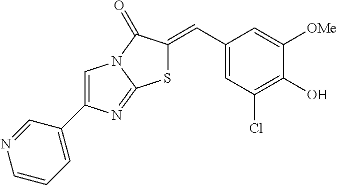 [SMILES: COc1cc(/C=c2\sc3nc(-c4cccnc4)cn3c2=O)cc(Cl)c1O] | 2-[1-(3-Chloro-4-hydroxy- 5-methoxy-phenyl)-meth- (Z)-ylidene]-6-(pyridin-3- yl)-imidazo[2,1-b]thiazol- 3-one | 0.0104 | 0.700 | 78 |
| 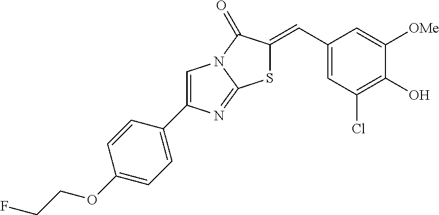 [SMILES: COc1cc(/C=c2\sc3nc(-c4ccc(OCCF)cc4)cn3c2=O)cc(Cl)c1O] | 2-[1-(3-Chloro-4-hydroxy- 5-methoxy-phenyl)-meth- (Z)-ylidene]-6-(4-(2- fluoroethoxy)phenyl)- imidazo[2,1-b]thiazol-3- one | 0.006 | 0.025 | 79 |
| 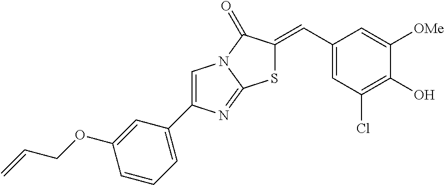 [SMILES: C=CCOc1cccc(-c2cn3c(=O)/c(=C/c4cc(Cl)c(O)c(OC)c4)sc3n2)c1] | 2-[1-(3-Chloro-4-hydroxy- 5-methoxy-phenyl)-meth- (Z)-ylidene]-6-(3- (allyloxy)phenyl)- imidazo[2,1-b]thiazol-3- one | 0.006 | 0.020 | 80 |
| 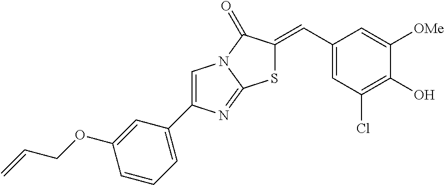 [SMILES: C=CCOc1cccc(-c2cn3c(=O)/c(=C/c4cc(Cl)c(O)c(OC)c4)sc3n2)c1] | 2-[1-(3-Chloro-4-hydroxy- 5-methoxy-phenyl)-meth- (Z)-ylidene]-6-(3- propoxyphenyl)- imidazo[2,1-b]thiazol-3- one | 0.011 | 1.498 | 81 |
| 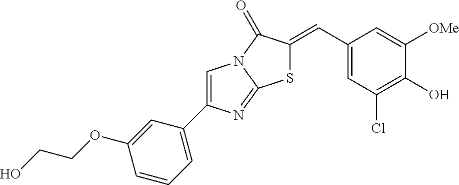 [SMILES: COc1cc(/C=c2\sc3nc(-c4cccc(OCCO)c4)cn3c2=O)cc(Cl)c1O] | 2-[1-(3-Chloro-4-hydroxy- 5-methoxy-phenyl)-meth- (Z)-ylidene]-6-(2- (hydroxyethoxy)phenyl)- imidazo[2,1-b]thiazol-3- one | 0.003 | 0.174 | 82 |
| 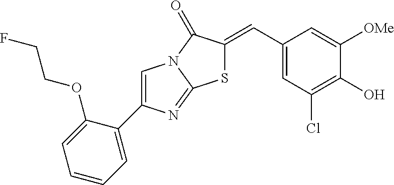 [SMILES: COc1cc(/C=c2\sc3nc(-c4ccccc4OCCF)cn3c2=O)cc(Cl)c1O] | 2-[1-(3-Chloro-4-hydroxy- 5-methoxy-phenyl)-meth- (Z)-ylidene]-6-(2-(2- fluoroethoxy)phenyl)- imidazo[2,1-b]thiazol-3- one | 0.173 | | 83 |
| 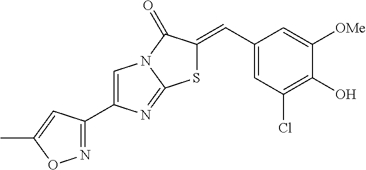 [SMILES: COc1cc(/C=c2\sc3nc(-c4cc(C)on4)cn3c2=O)cc(Cl)c1O] | 2-[1-(3-Chloro-4-hydroxy- 5-methoxy-phenyl)-meth- (Z)-ylidene]-6-(5-methyl- isoxazol-3-yl)-imidazo[2,1- b]thiazol-3-one | 0.018 | 0.990 | 84 |
| 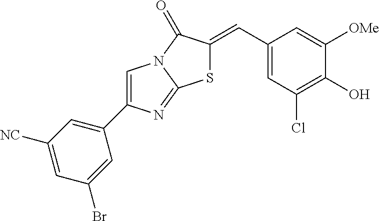 [SMILES: COc1cc(/C=c2\sc3nc(-c4cc(Br)cc(C#N)c4)cn3c2=O)cc(Cl)c1O] | 3-Bromo-5-{2-[1-(3- chloro-4-hydroxy-5- methoxy-phenyl)-meth-(Z)- ylidene]-3-oxo-2,3- dihydro-imidazo[2,1- b]thiazol-6-yl}-benzonitrile | 0.0008 | 1.730 | 85 |
| 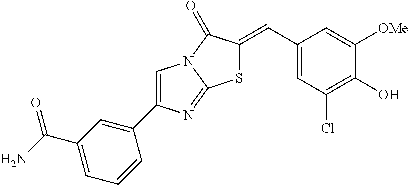 [SMILES: COc1cc(/C=c2\sc3nc(-c4cccc(C(N)=O)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzamide | 0.001 | 1.242 | 86 |
| 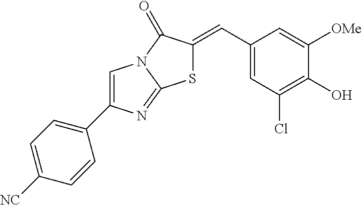 [SMILES: COc1cc(/C=c2\sc3nc(-c4ccc(C#N)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzonitrile | 0.0003 | 0.006 | 87 |
| 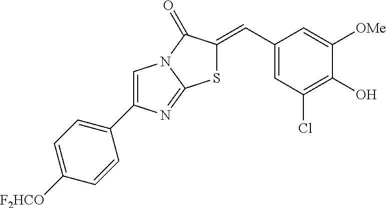 [SMILES: COc1cc(/C=c2\sc3nc(-c4ccc(OC(F)F)cc4)cn3c2=O)cc(Cl)c1O] | 2-[1-(3-Chloro-4-hydroxy- 5-methoxy-phenyl)-meth- (Z)-ylidene]-6-(4- (difluoromethoxy)phenyl)- imidazo[2,1-b]thiazol-3- one | 0.0006 | 0.014 | 88 |
| 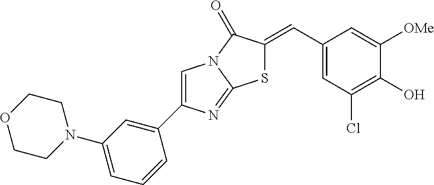 [SMILES: COc1cc(/C=c2\sc3nc(-c4cccc(N5CCOCC5)c4)cn3c2=O)cc(Cl)c1O] | 2-[1-(3-Chloro-4-hydroxy- 5-methoxy-phenyl)-meth- (Z)-ylidene]-6-(3- morpholinophenyl)- imidazo[2,1-b]thiazol-3- one | 0.024 | 0.356 | 89 |
| 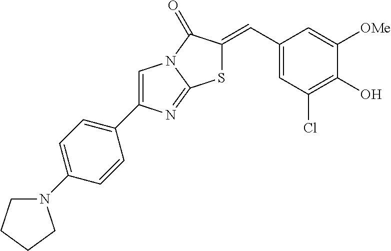 [SMILES: COc1cc(/C=c2\sc3nc(-c4ccc(N5CCCC5)cc4)cn3c2=O)cc(Cl)c1O] | 2-[1-(3-Chloro-4-hydroxy- 5-methoxy-phenyl)-meth- (Z)-ylidene]-6-(4- (pyrrolidin-1-yl)phenyl)- imidazo[2,1-b]thiazol-3- one | 0.037 | 0.016 | 90 |
| 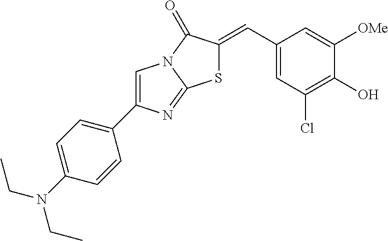 [SMILES: CCN(CC)c1ccc(-c2cn3c(=O)/c(=C/c4cc(Cl)c(O)c(OC)c4)sc3n2)cc1] | 2-[1-(3-Chloro-4-hydroxy- 5-methoxy-phenyl)-meth- (Z)-ylidene]-6-(4- (diethylamino)phenyl)- imidazo[2,1-b]thiazol-3- one | 0.020 | 0.184 | 91 |
| 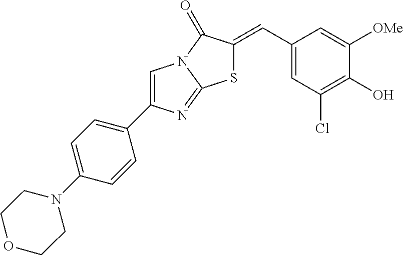 [SMILES: COc1cc(/C=c2\sc3nc(-c4ccc(N5CCOCC5)cc4)cn3c2=O)cc(Cl)c1O] | 2-[1-(3-Chloro-4-hydroxy- 5-methoxy-phenyl)-meth- (Z)-ylidene]-6-(4- morpholinophenyl)- imidazo[2,1-b]thiazol-3- one | 0.039 | 0.482 | 92 |
| 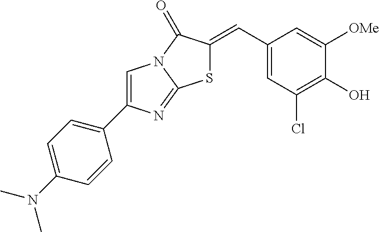 [SMILES: COc1cc(/C=c2\sc3nc(-c4ccc(N(C)C)cc4)cn3c2=O)cc(Cl)c1O] | 2-[1-(3-Chloro-4-hydroxy- 5-methoxy-phenyl)-meth- (Z)-ylidene]-6-(4- (dimethylamino)phenyl)- imidazo[2,1-b]thiazol-3- one | 0.030 | 0.029 | 93 |
| 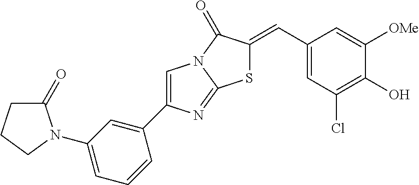 [SMILES: COc1cc(/C=c2\sc3nc(-c4cccc(N5CCCC5=O)c4)cn3c2=O)cc(Cl)c1O] | 2-[1-(3-Chloro-4-hydroxy- 5-methoxy-phenyl)-meth- (Z)-ylidene]-6-(3-(2-oxo- pyrrolidin-1-yl)-phenyl]- imidazo[2,1-b]thiazol-3- one | 0.042 | 3.892 | 94 |
| 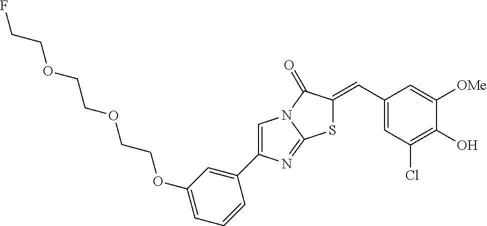 [SMILES: COc1cc(/C=c2\sc3nc(-c4cccc(OCCOCCOCCF)c4)cn3c2=O)cc(Cl)c1O] | 2-[1-(3-Chloro-4-hydroxy- 5-methoxy-phenyl)-meth- (Z)-ylidene]-6-(3-{2-[2-(2- fluoro-ethoxy)-ethoxy]- ethoxy}-phenyl)- imidazo[2,1-b]thiazol-3- one | 0.028 | 2.147 | 95 |
| 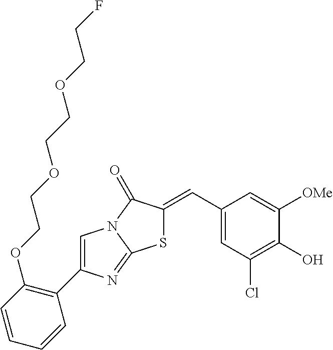 [SMILES: COc1cc(/C=c2\sc3nc(-c4ccccc4OCCOCCOCCF)cn3c2=O)cc(Cl)c1O] | 2-[1-(3-Chloro-4-hydroxy- 5-methoxy-phenyl)-meth- (Z)-ylidene]-6-(2-{2-[2-(2- fluoro-ethoxy)-ethoxy]- ethoxy}-phenyl)- imidazo[2,1-b]thiazol-3- one | 0.102 | | 96 |
| 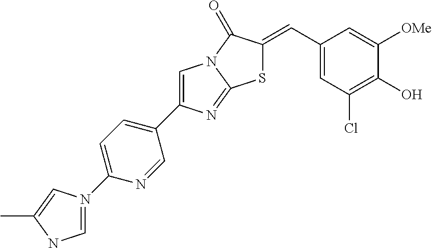 | 2-[1-(3-Chloro-4-hydroxy- 5-methoxy-phenyl)-meth- (Z)-ylidene]-6-[6-(4- methyl-3H-imidazol-1-yl)- pyridin-3-yl]-imidazo[2,1- b]thiazol-3-one | 0.0017 | 0.185 | 97 |
| 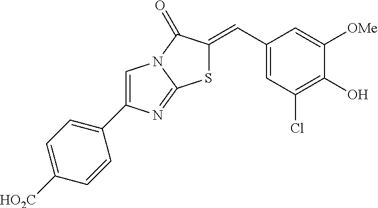 [SMILES: COc1cc(/C=c2\sc3nc(-c4ccc(C(=O)O)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzoic acid | 0.021 | 1.846 | 98 |
| 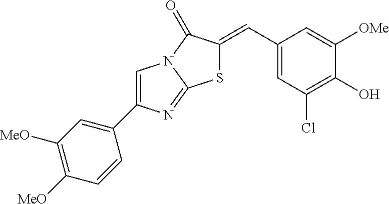 [SMILES: COc1ccc(-c2cn3c(=O)/c(=C/c4cc(Cl)c(O)c(OC)c4)sc3n2)cc1OC] | 2-[1-(3-Chloro-4-hydroxy- 5-methoxy-phenyl)-meth- (Z)-ylidene]-6-(3,4- dimethoxy-phenyl)- imidazo[2,1-b]thiazol-3- one | 0.014 | 0.325 | 99 |
| 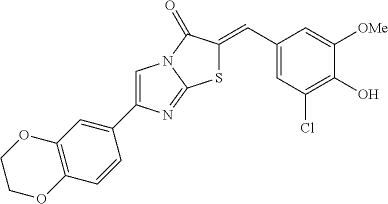 [SMILES: COc1cc(/C=c2\sc3nc(-c4ccc5c(c4)OCCO5)cn3c2=O)cc(Cl)c1O] | 2-[1-(3-Chloro-4-hydroxy- 5-methoxy-phenyl)-meth- (Z)-ylidene]-6-(2,3- dihydro-benzo[1,4]dioxin- 6-yl)-imidazo[2,1- b]thiazol-3-one | 0.009 | 1.061 | 100 |
| 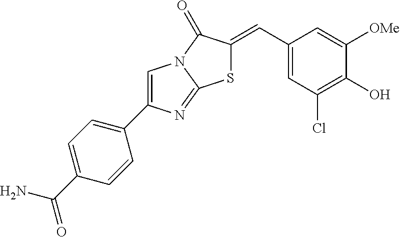 [SMILES: COc1cc(/C=c2\sc3nc(-c4ccc(C(N)=O)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzamide | 0.002 | 0.792 | 101 |
| 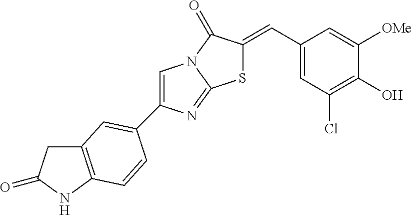 [SMILES: COc1cc(/C=c2\sc3nc(-c4ccc5c(c4)CC(=O)N5)cn3c2=O)cc(Cl)c1O] | 5-{2-[1-(3-Chloro-4- hydroxy-5-methoxy- phenyl)-meth-(Z)-ylidene]- 3-oxo-2,3-dihydro- imidazo[2,1-b]thiazol-6- yl}-1,3-dihydro-indol-2- one | 0.012 | 0.226 | 102 |
| 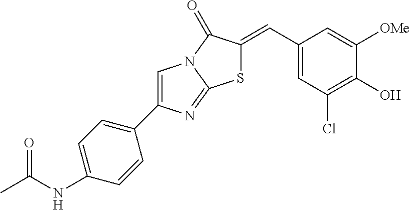 [SMILES: COc1cc(/C=c2\sc3nc(-c4ccc(NC(C)=O)cc4)cn3c2=O)cc(Cl)c1O] | N-(4-{2-[1-(3-Chloro-4- hydroxy-5-methoxy- phenyl)-meth-(Z)-ylidene]- 3-oxo-2,3-dihydro- imidazo[2,1-b]thiazol-6- yl}-phenyl)-acetamide | 0.024 | 1.165 | 103 |
| 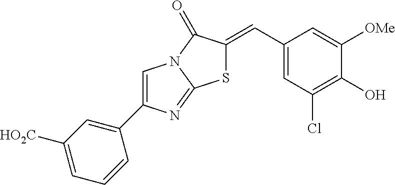 [SMILES: COc1cc(/C=c2\sc3nc(-c4cccc(C(=O)O)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzoic acid | 0.019 | >10 | 104 |
| 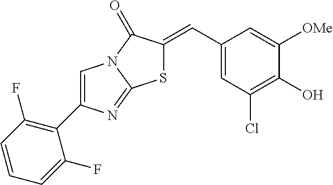 [SMILES: COc1cc(/C=c2\sc3nc(-c4c(F)cccc4F)cn3c2=O)cc(Cl)c1O] | 2-[1-(3-Chloro-4-hydroxy- 5-methoxy-phenyl)-meth- (Z)-ylidene]-6-(2,6- difluoro-phenyl)- imidazo[2,1-b]thiazol-3- one | 0.013 | 5.551 | 105 |
| 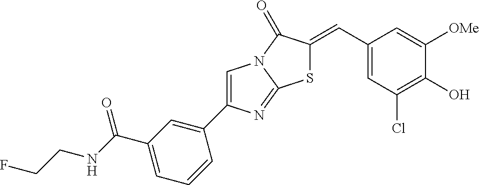 [SMILES: COc1cc(/C=c2\sc3nc(-c4cccc(C(=O)NCCF)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(2-fluoro-ethyl)- benzamide | 0.008 | 1.080 | 106 |
| 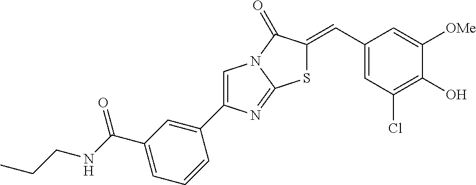 [SMILES: CCCNC(=O)c1cccc(-c2cn3c(=O)/c(=C/c4cc(Cl)c(O)c(OC)c4)sc3n2)c1] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-propyl-benzamide | 0.003 | 2.630 | 107 |
| 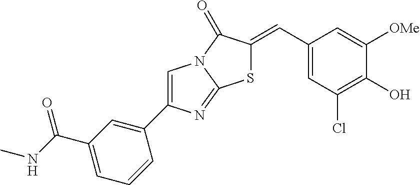 [SMILES: CNC(=O)c1cccc(-c2cn3c(=O)/c(=C/c4cc(Cl)c(O)c(OC)c4)sc3n2)c1] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-methyl-benzamide | 0.004 | 0.395 | 108 |
| 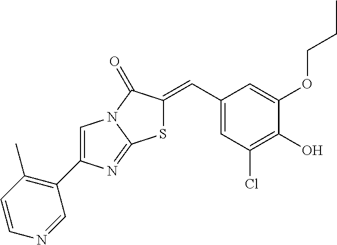 [SMILES: CCCOc1cc(/C=c2\sc3nc(-c4cnccc4C)cn3c2=O)cc(Cl)c1O] | 2-[1-(3-Chloro-4-hydroxy- 5-propoxy-phenyl)-meth- (Z)-ylidene]-6-(4-methyl- pyridin-3-yl)-imidazo[2,1- b]thiazol-3-one | 0.033 | 4.917 | 109 |
| 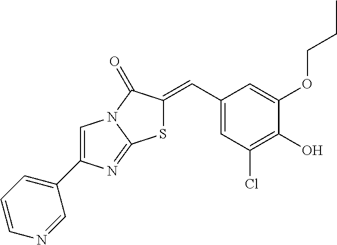 [SMILES: CCCOc1cc(/C=c2\sc3nc(-c4cccnc4)cn3c2=O)cc(Cl)c1O] | 2-[1-(3-Chloro-4-hydroxy- 5-propoxy-phenyl)-meth- (Z)-ylidene]-6-pyridin-3- yl-imidazo[2,1-b]thiazol-3- one | 0.017 | 0.425 | 110 |
| 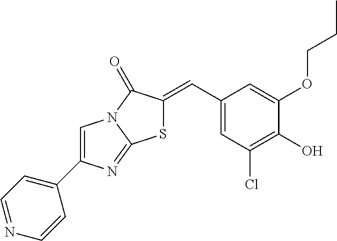 [SMILES: CCCOc1cc(/C=c2\sc3nc(-c4ccncc4)cn3c2=O)cc(Cl)c1O] | 2-[1-(3-Chloro-4-hydroxy- 5-propoxy-phenyl)-meth- (Z)-ylidene]-6-pyridin-4- yl-imidazo[2,1-b]thiazol-3- one | 0.017 | 1.449 | 111 |
| 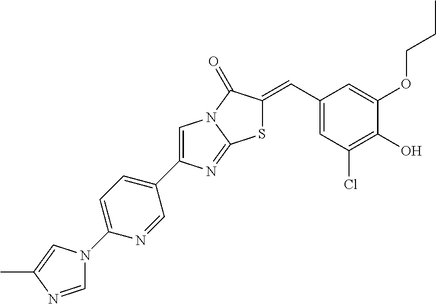 [SMILES: CCCOc1cc(/C=c2\sc3nc(-c4ccc(-n5cnc(C)c5)nc4)cn3c2=O)cc(Cl)c1O] | 2-[1-(3-Chloro-4-hydroxy- 5-propoxy-phenyl)-meth- (Z)-ylidene]-6-[6-(4- methyl-imidazol-1-yl)- pyridin-3-yl]-imidazo[2,1- b]thiazol-3-one | 0.008 | 0.493 | 112 |
| 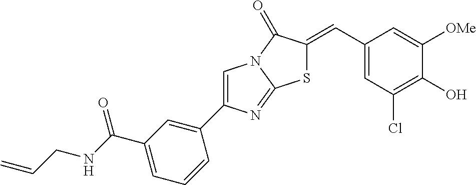 [SMILES: C=CCNC(=O)c1cccc(-c2cn3c(=O)/c(=C/c4cc(Cl)c(O)c(OC)c4)sc3n2)c1] | N-Allyl-3-{2-[1-(3-chloro- 4-hydroxy-5-methoxy- phenyl)-meth-(Z)-ylidene]- 3-oxo-2,3-dihydro- imidazo[2,1-b]thiazol-6- yl}-benzamide | 0.016 | 1.164 | 113 |
| 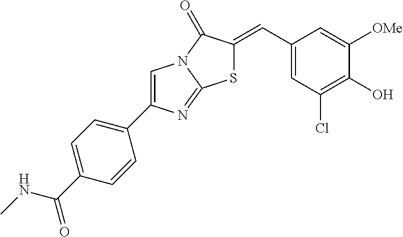 [SMILES: CNC(=O)c1ccc(-c2cn3c(=O)/c(=C/c4cc(Cl)c(O)c(OC)c4)sc3n2)cc1] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-methyl-benzamide | 0.006 | 0.352 | 114 |
| 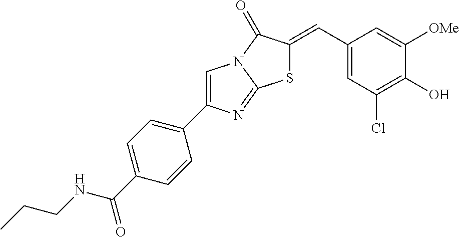 [SMILES: CCCNC(=O)c1ccc(-c2cn3c(=O)/c(=C/c4cc(Cl)c(O)c(OC)c4)sc3n2)cc1] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-propyl-benzamide | 0.002 | 0.356 | 115 |
| 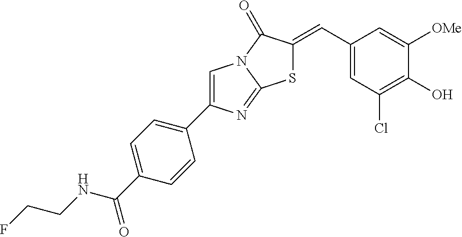 [SMILES: COc1cc(/C=c2\sc3nc(-c4ccc(C(=O)NCCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(2-fluoro-ethyl)- benzamide | 0.003 | 0.820 | 116 |
| 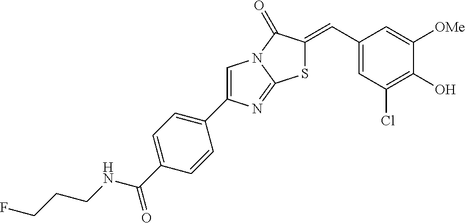 [SMILES: COc1cc(/C=c2\sc3nc(-c4ccc(C(=O)NCCCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(3-fluoro-propyl)- benzamide | 0.005 | 0.643 | 117 |
| 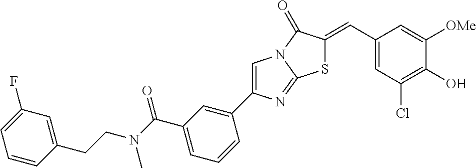 [SMILES: COc1cc(/C=c2\sc3nc(-c4cccc(C(=O)N(C)CCc5cccc(F)c5)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-[2-(3-fluoro-phenyl)- ethyl]-N-methyl-benzamide | 0.0129 | >10 | 118 |
| 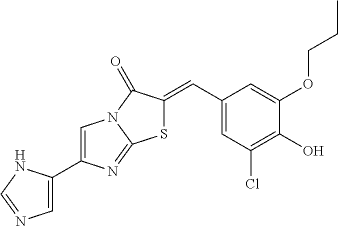 [SMILES: CCCOc1cc(/C=c2\sc3nc(-c4cnc[nH]4)cn3c2=O)cc(Cl)c1O] | 2-[1-(3-Chloro-4-hydroxy- 5-propoxy-phenyl)-meth- (Z)-ylidene]-6-(3H- imidazol-4-yl)- imidazo[2,1-b]thiazol-3- one | 0.060 | 3.117 | 119 |
| 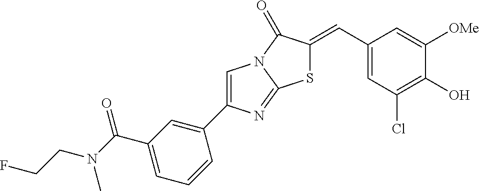 [SMILES: COc1cc(/C=c2\sc3nc(-c4cccc(C(=O)N(C)CCF)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(2-fluoro-ethyl)-N- methyl-benzamide | 0.049 | | 120 |
| 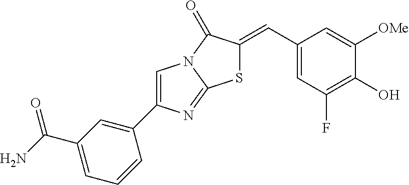 [SMILES: COc1cc(/C=c2\sc3nc(-c4cccc(C(N)=O)c4)cn3c2=O)cc(F)c1O] | 3-{2-[1-(3-Fluoro-4- hydroxy-5-methoxy- phenyl)-meth-(Z)-ylidene]- 3-oxo-2,3-dihydro- imidazo[2,1-b]thiazol-6- yl}-benzamide | 0.019 | 0.591 | 121 |
| 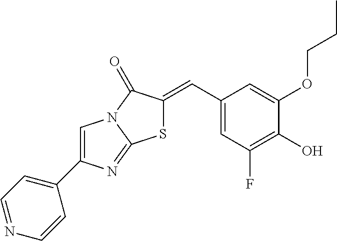 [SMILES: CCCOc1cc(/C=c2\sc3nc(-c4ccncc4)cn3c2=O)cc(F)c1O] | 2-[1-(3-Fluoro-4-hydroxy- 5-propoxy-phenyl)-meth- (Z)-ylidene]-6-pyridin-4- yl-imidazo[2,1-b]thiazol-3- one | 0.0235 | 2.673 | 122 |
| 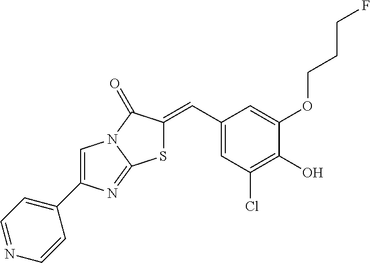 [SMILES: O=c1/c(=C/c2cc(Cl)c(O)c(OCCCF)c2)sc2nc(-c3ccncc3)cn12] | 2-[1-(3-Chloro-5-(3- fluoropropoxy)-4-hydroxy- phenyl)-meth-(Z)-ylidene]- 6-pyridin-4-yl-imidazo[2,1- b]thiazol-3-one | 0.012 | 1.061 | 123 |
| 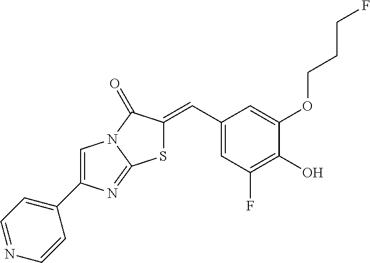 [SMILES: O=c1/c(=C/c2cc(F)c(O)c(OCCCF)c2)sc2nc(-c3ccncc3)cn12] | 2-[1-(3-Fluoro-5-(3- fluoropropoxy)-4-hydroxy- phenyl)-meth-(Z)-ylidene]- 6-pyridin-4-yl-imidazo[2,1- b]thiazol-3-one | 0.017 | 1.225 | 124 |
| 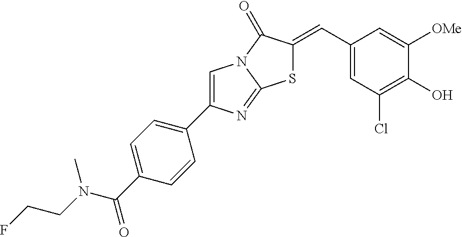 [SMILES: COc1cc(/C=c2\sc3nc(-c4ccc(C(=O)N(C)CCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(2-fluoro-ethyl)-N- methyl-benzamide | 0.017 | 0.621 | 125 |
| 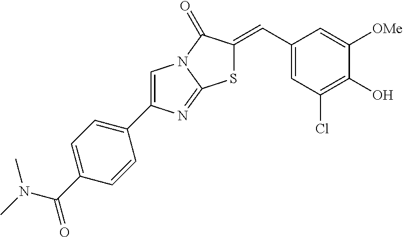 [SMILES: COc1cc(/C=c2\sc3nc(-c4ccc(C(=O)N(C)C)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N,N-dimethyl- benzamide | 0.028 | 0.550 | 126 |
| 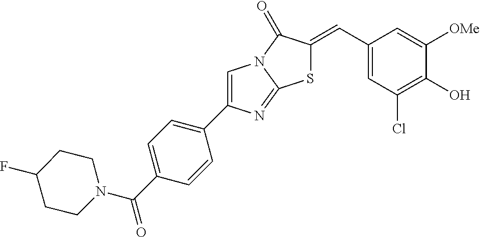 [SMILES: COc1cc(/C=c2\sc3nc(-c4ccc(C(=O)N5CCC(F)CC5)cc4)cn3c2=O)cc(Cl)c1O] | 2-[1-(3-Chloro-4-hydroxy- 5-methoxy-phenyl)-meth- (Z)-ylidene]-6-[4-(4-fluoro- piperidine-1-carbonyl)- phenyl]-imidazo[2,1- b]thiazol-3-one | 0.006 | 0.118 | 127 |
| 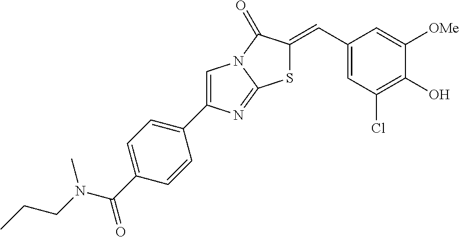 [SMILES: CCCN(C)C(=O)c1ccc(-c2cn3c(=O)/c(=C/c4cc(Cl)c(O)c(OC)c4)sc3n2)cc1] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-methyl-N-propyl- benzamide | 0.009 | 0.218 | 128 |
| 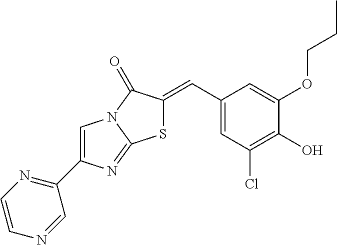 [SMILES: CCCOc1cc(/C=c2\sc3nc(-c4cnccn4)cn3c2=O)cc(Cl)c1O] | 2-[1-(3-Chloro-4-hydroxy- 5-propoxy-phenyl)-meth- (Z)-ylidene]-6-pyrazin-2- yl-imidazo[2,1-b]thiazol-3- one | 0.064 | 2.216 | 129 |
| 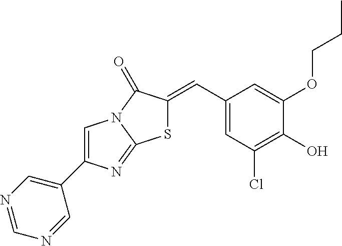 [SMILES: CCCOc1cc(/C=c2\sc3nc(-c4cncnc4)cn3c2=O)cc(Cl)c1O] | 2-[1-(3-Chloro-4-hydroxy- 5-propoxy-phenyl)-meth- (Z)-ylidene]-6-pyrimidin-5- yl-imidazo[2,1-b]thiazol-3- one | 0.067 | 1.945 | 130 |
| 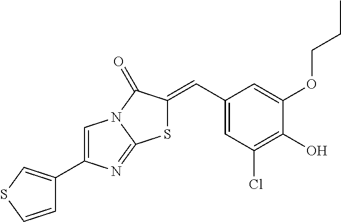 [SMILES: CCCOc1cc(/C=c2\sc3nc(-c4ccsc4)cn3c2=O)cc(Cl)c1O] | 2-[1-(3-Chloro-4-hydroxy- 5-propoxy-phenyl)-meth- (Z)-ylidene]-6-thiophen-3- yl-imidazo[2,1-b]thiazol-3- one | 0.026 | 0.979 | 131 |
| 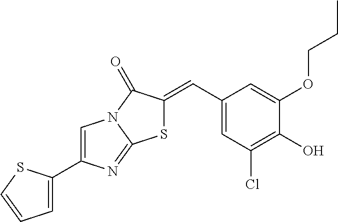 [SMILES: CCCOc1cc(/C=c2\sc3nc(-c4cccs4)cn3c2=O)cc(Cl)c1O] | 2-[1-(3-Chloro-4-hydroxy- 5-propoxy-phenyl)-meth- (Z)-ylidene]-6-thiophen-2- yl-imidazo[2,1-b]thiazol-3- one | 0.041 | 0.718 | 132 |
| 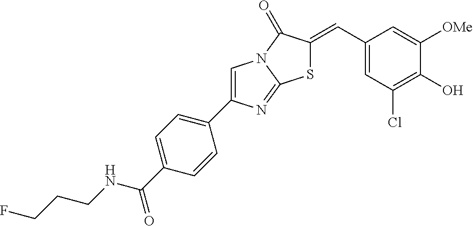 [SMILES: COc1cc(/C=c2\sc3nc(-c4ccc(C(=O)NCCCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(3-fluoropropoxy)- benzamide | 0.002 | 0.086 | 133 |
| 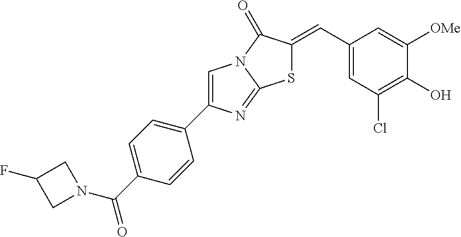 [SMILES: COc1cc(/C=c2\sc3nc(-c4ccc(C(=O)N5CC(F)C5)cc4)cn3c2=O)cc(Cl)c1O] | 2-[1-(3-Chloro-4-hydroxy- 5-methoxy-phenyl)-meth- (Z)-ylidene]-6-[4-(3-fluoro- azetidine-1-carbonyl)- phenyl]-imidazo[2,1- b]thiazol-3-one | 0.002 | 0.133 | 134 |
| 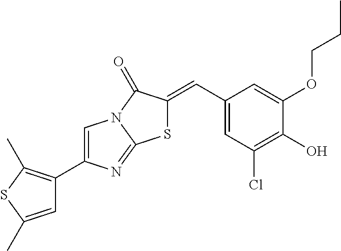 [SMILES: CCCOc1cc(/C=c2\sc3nc(-c4cc(C)sc4C)cn3c2=O)cc(Cl)c1O] | 2-[1-(3-Chloro-4-hydroxy- 5-propoxy-phenyl)-meth- (Z)-ylidene]-6-(2,4- dimethylthiophen-3-yl)- imidazo[2,1-b]thiazol-3- one | 0.043 | 1.058 | 135 |
| 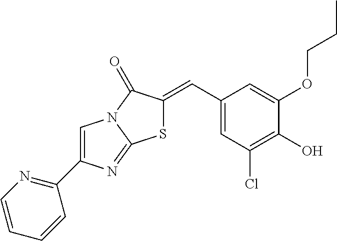 [SMILES: CCCOc1cc(/C=c2\sc3nc(-c4ccccn4)cn3c2=O)cc(Cl)c1O] | 2-[1-(3-Chloro-4-hydroxy- 5-propoxy-phenyl)-meth- (Z)-ylidene]-6-pyridin-2- yl-imidazo[2,1-b]thiazol-3- one | 0.030 | 0.293 | 136 |
| 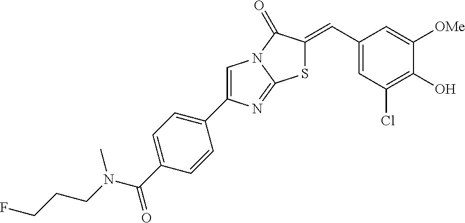 [SMILES: COc1cc(/C=c2\sc3nc(-c4ccc(C(=O)N(C)CCCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-(3-fluoropropoxy)- N-methyl-benzamide | 0.007 | 0.332 | 137 |
| 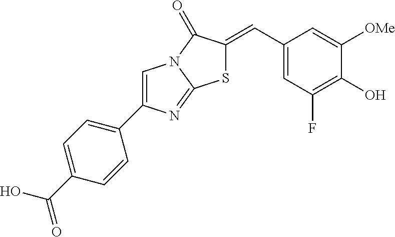 [SMILES: COc1cc(/C=c2\sc3nc(-c4ccc(C(=O)O)cc4)cn3c2=O)cc(F)c1O] | 4-{2-[1-(3-Fluoro-4- hydroxy-5-methoxy- phenyl)-meth-(Z)-ylidene]- 3-oxo-2,3-dihydro- imidazo[2,1-b]thiazol-6- yl}-benzoic acid | 0.014 | 0.898 | 138 |
| 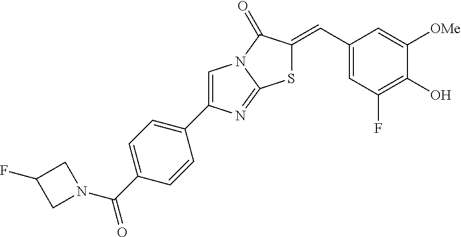 [SMILES: COc1cc(/C=c2\sc3nc(-c4ccc(C(=O)N5CC(F)C5)cc4)cn3c2=O)cc(F)c1O] | 6-[4-(3-Fluoro-azetidine-1- carbonyl)-phenyl]-2-[1-(3- fluoro-4-hydroxy-5- methoxy-phenyl)-meth-(Z)- ylidene]-imidazo[2,1- b]thiazol-3-one | | | 139 |
| 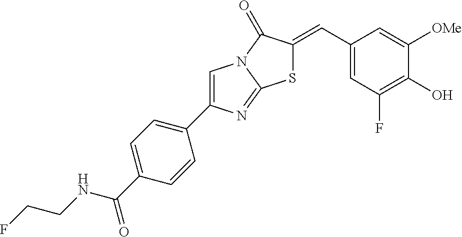 [SMILES: COc1cc(/C=c2\sc3nc(-c4ccc(C(=O)NCCF)cc4)cn3c2=O)cc(F)c1O] | 4-{2-[1-(3-Fluoro-4- hydroxy-5-methoxy- phenyl)-meth-(Z)-ylidene]- 3-oxo-2,3-dihydro- imidazo[2,1-b]thiazol-6- yl}-N-(2-fluoroethoxy)- benzamide | | | 140 |
| 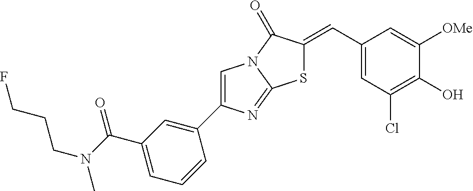 [SMILES: COc1cc(/C=c2\sc3nc(-c4cccc(C(=O)N(C)CCCF)c4)cn3c2=O)cc(Cl)c1O] | 3-{2-[1-(3-Fluoro-4- hydroxy-5-methoxy- phenyl)-meth-(Z)-ylidene]- 3-oxo-2,3-dihydro- imidazo[2,1-b]thiazol-6- yl}-N-(3-fluoropropoxy)- N-methyl-benzamide | | | 141 |
| 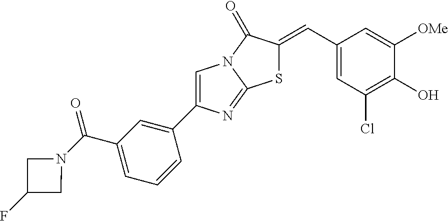 [SMILES: COc1cc(/C=c2\sc3nc(-c4cccc(C(=O)N5CC(F)C5)c4)cn3c2=O)cc(Cl)c1O] | 2-[1-(3-Chloro-4-hydroxy- 5-methoxy-phenyl)-meth- (Z)-ylidene]-6-[3-(3-fluoro- azetidine-1-carbonyl)- phenyl]-imidazo[2,1- b]thiazol-3-one | | | 142 |
| 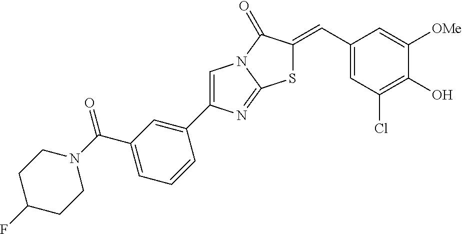 [SMILES: COc1cc(/C=c2\sc3nc(-c4cccc(C(=O)N5CCC(F)CC5)c4)cn3c2=O)cc(Cl)c1O] | 2-[1-(3-Chloro-4-hydroxy- 5-methoxy-phenyl)-meth- (Z)-ylidene]-6-[3-(4- fluoro-piperidine-1-carbon- phenyl]-imidazo[2,1- b]thiazol-3-one | | | 143 |
| 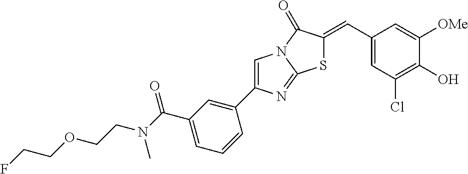 [SMILES: COc1cc(/C=c2\sc3nc(-c4cccc(C(=O)N(C)CCOCCF)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-N-[2-(2-fluoro-ethoxy)- ethyl]-N-methyl-benzamide | | | 144 |
| 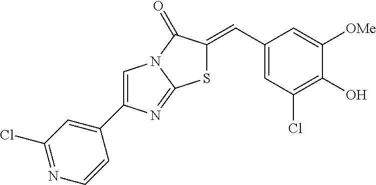 [SMILES: COc1cc(/C=c2\sc3nc(-c4ccnc(Cl)c4)cn3c2=O)cc(Cl)c1O] | 2-[1-(3-Chloro-4-hydroxy- 5-methoxy-phenyl)-meth- (Z)-ylidene]-6-(2-chloro- pyridin-4-yl)-imidazo[2,1- b]thiazol-3-one | | | 145 |
| 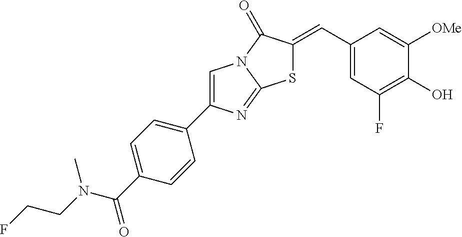 [SMILES: COc1cc(/C=c2\sc3nc(-c4ccc(C(=O)N(C)CCF)cc4)cn3c2=O)cc(F)c1O] | 4-{2-[1-(3-Fluoro-4- hydroxy-5-methoxy- phenyl)-meth-(Z)-ylidene]- 3-oxo-2,3-dihydro- imidazo[2,1-b]thiazol-6- yl}-N-(2-fluoroethoxy)-N- methyl-benzamide | 0.048 | 1.010 | 146 |
| 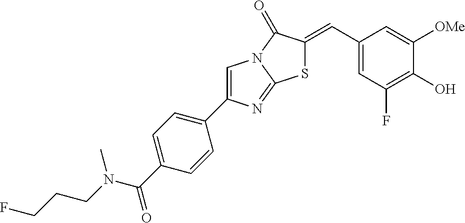 [SMILES: COc1cc(/C=c2\sc3nc(-c4ccc(C(=O)N(C)CCCF)cc4)cn3c2=O)cc(F)c1O] | 4-{2-[1-(3-Fluoro-4- hydroxy-5-methoxy- phenyl)-meth-(Z)-ylidene]- 3-oxo-2,3-dihydro- imidazo[2,1-b]thiazol-6- yl}-N-(2-fluoropropoxy)- N-methyl-benzamide | 0.041 | 0.448 | 147 |
| 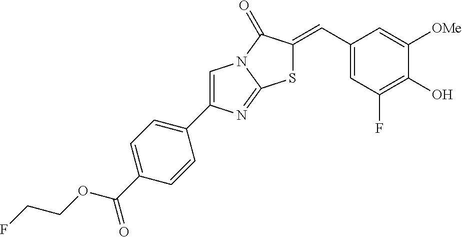 [SMILES: COc1cc(/C=c2\sc3nc(-c4ccc(C(=O)OCCF)cc4)cn3c2=O)cc(F)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzoic acid 2-fluoro- ethyl ester | 0.004 | 0.038 | 148 |
| 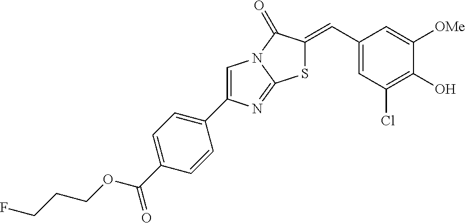 [SMILES: COc1cc(/C=c2\sc3nc(-c4ccc(C(=O)OCCCF)cc4)cn3c2=O)cc(Cl)c1O] | 4-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzoic acid 3-fluoro- propyl ester | 0.002 | 0.168 | 149 |
| 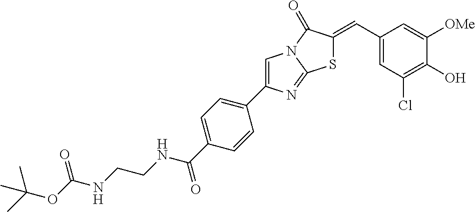 [SMILES: COc1cc(/C=c2\sc3nc(-c4ccc(C(=O)NCCNC(=O)OC(C)(C)C)cc4)cn3c2=O)cc(Cl)c1O] | [2-(4-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzoylamino)-ethyl]- carbamic acid ter-butyl ester | 0.0018 | 0.118 | 150 |
| 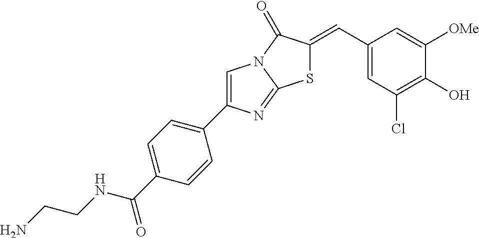 [SMILES: COc1cc(/C=c2\sc3nc(-c4ccc(C(=O)NCCN)cc4)cn3c2=O)cc(Cl)c1O] | N-(2-Amino-ethyl)-4-{2- [1-(3-chloro-4-hydroxy-5- methoxy-phenyl)-meth-(Z)- ylidene]-3-oxo-2,3- dihydro-imidazo[2,1- b]thiazol-6-yl}-benzamide | 0.012 | 0.024 | 151 |
| 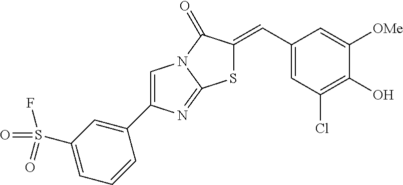 [SMILES: COc1cc(/C=c2\sc3nc(-c4cccc(S(=O)(=O)F)c4)cn3c2=O)cc(Cl)c1O] | 3-{2-[1-(3-Chloro-4- hydroxy-5-methoxy- phenyl)-meth-(Z)-ylidene]- 3-oxo-2,3-dihydro- imidazo[2,1-b]thiazol-6- yl}-benzenesulfonyl fluoride | 0.007 | 0.019 | 152 |
| 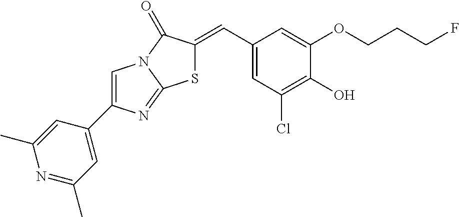 [SMILES: Cc1cc(-c2cn3c(=O)/c(=C/c4cc(Cl)c(O)c(OCCCF)c4)sc3n2)cc(C)n1] | 2-[1-[3-Chloro-5-(3-fluoro- propoxy)-4-hydroxy- phenyl]-meth-(Z)-ylidene]- 6-(2,6-dimethyl-pyridin-4- yl)-imidazo[2,1-b]thiazol- 3-one | 0.046 | 0.605 | 153 |
| 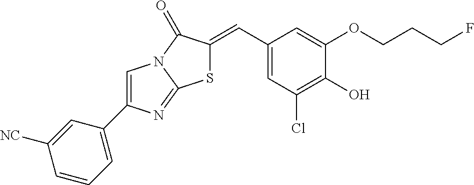 [SMILES: N#Cc1cccc(-c2cn3c(=O)/c(=C/c4cc(Cl)c(O)c(OCCCF)c4)sc3n2)c1] | 3-{2-[1-[3-Chloro-5-(3- fluoro-propoxy)-4- hydroxy-phenyl]-meth-(Z)- ylidene]-3-oxo-2,3- dihydro-imidazo[2,1- b]thiazol-6-yl}-benzonitrile | | | 154 |

### EXAMPLE 1

### 2-[1-(4-Hydroxy-3,5-dimethoxy-phenyl)-meth-(Z)-ylidene]-6-pyridin-4-yl-imidazo[2,1-b]thiazol-3-one

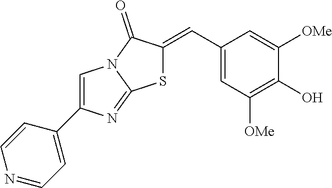 [SMILES: COc1cc(/C=c2\sc3nc(-c4ccncc4)cn3c2=O)cc(OC)c1O]

To a mixture of 5-(pyridin-4-yl)-1H-imidazole-2-thiol (70 mg, 395 μmol), 4-hydroxy-3,5-dimethoxybenzaldehyde (72.0 mg, 395 μmol), chloroacetic acid (37.3 mg, 26.6 μl, 395 μmol) and sodium acetate (64.8 mg, 790 μmol) was added acetic acid (1.5 mL). The resulting suspension was warmed to 130° C. and stirred overnight at this temperature in a sealed tube. After cooling to ambient temperature water (2 mL) was added and the reaction mixture was centrifuged. The supernatant was removed and the residue washed with water (2 mL). Suspension in boiling ethanol (2 mL) was followed by filtering. Washing of the remaining solid with ethanol (2 mL) afforded the title compound (20 mg, 13%) as a yellow solid. MS m/e: 382.1 [M+H]⁺

### EXAMPLE 2

### 6-(4-Chloro-phenyl)-2-[1-(4-hydroxy-3,5-dimethoxy-phenyl)-meth-(Z)-ylidene]-imidazo[2,1-b]thiazol-3-one

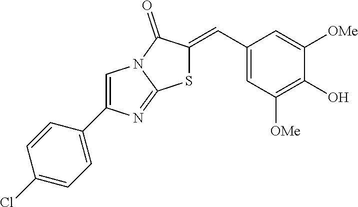 [SMILES: COc1cc(/C=c2\sc3nc(-c4ccc(Cl)cc4)cn3c2=O)cc(OC)c1O]

In analogy to the experimental procedure of example 1) 5-(4-chlorophenyl)-1H-imidazole-2-thiol instead of 5-(pyridin-4-yl)-1H-imidazole-2-thiol was converted into the title compound (23 mg, 17%) which was obtained as a yellow solid. MS m/e: 415.1 [M+H]⁺

### EXAMPLE 3

### 6-(4-Chloro-phenyl)-2-[1-(4-hydroxy-3-methoxy-phenyl)-meth-(Z)-ylidene]-imidazo[2,1-b]thiazol-3-one

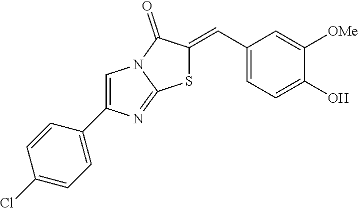 [SMILES: COc1cc(/C=c2\sc3nc(-c4ccc(Cl)cc4)cn3c2=O)ccc1O]

In analogy to the experimental procedure of example 1) 5-(4-chlorophenyl)-1H-imidazole-2-thiol was converted using 4-hydroxy-3-methoxybenzaldehyde instead of 4-hydroxy-3,5-dimethoxybenzaldehyde into the title compound (82 mg, 45%) which was obtained as a light yellow solid. MS m/e: 385.0 [M+H]⁺

### EXAMPLE 4

### 6-(4-Chloro-phenyl)-2-[1-(3-fluoro-4-hydroxy-5-methoxy-phenyl)-meth-(Z)-ylidene]-imidazo[2,1-b]thiazol-3-one

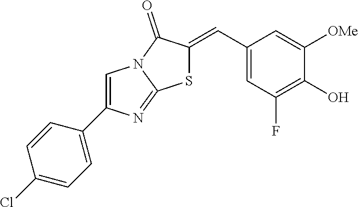 [SMILES: COc1cc(/C=c2\sc3nc(-c4ccc(Cl)cc4)cn3c2=O)cc(F)c1O]

In analogy to the experimental procedure of example 1) 5-(4-chlorophenyl)-1H-imidazole-2-thiol was converted using 3-fluoro-4-hydroxy-5-methoxybenzaldehyde instead of 4-hydroxy-3,5-dimethoxybenzaldehyde into the title compound (64 mg, 34%) which was obtained as a light yellow solid. MS m/e: 403.0 [M+H]⁺

### EXAMPLE 5

### 6-(4-Chloro-phenyl)-2-[1-(3-ethoxy-4-hydroxy-phenyl)-meth-(Z)-ylidene]-imidazo[2,1-b]thiazol-3-one

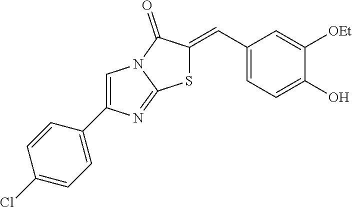 [SMILES: CCOc1cc(/C=c2\sc3nc(-c4ccc(Cl)cc4)cn3c2=O)ccc1O]

In analogy to the experimental procedure of example 1) 5-(4-chlorophenyl)-1H-imidazole-2-thiol was converted using 3-ethoxy-4-hydroxy-benzaldehyde instead of 4-hydroxy-3,5-dimethoxybenzaldehyde into the title compound (58 mg, 31%) which was obtained as a light yellow solid. MS m/e: 399.1 [M+H]⁺

### EXAMPLE 6

### 6-(4-Chloro-phenyl)-2-[1-(3-chloro-4-hydroxy-5-methoxy-phenyl)-meth-(Z)-ylidene]-imidazo[2,1-b]thiazol-3-one

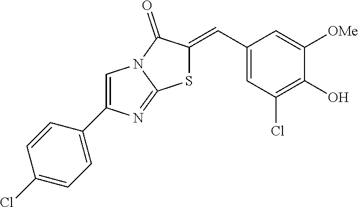 [SMILES: COc1cc(/C=c2\sc3nc(-c4ccc(Cl)cc4)cn3c2=O)cc(Cl)c1O]

In analogy to the experimental procedure of example 1) 5-(4-chlorophenyl)-1H-imidazole-2-thiol was converted using 3-chloro-4-hydroxy-5-methoxybenzaldehyde instead of 4-hydroxy-3,5-dimethoxybenzaldehyde into the title compound (75 mg, 38%) which was obtained as a light brown solid. MS m/e: 419.0 [M+H]⁺

### EXAMPLE 7

### 6-(4-Chloro-phenyl)-2-[1-(3-bromo-4-hydroxy-5-methoxy-phenyl)-meth-(Z)-ylidene]-imidazo[2,1-b]thiazol-3-one

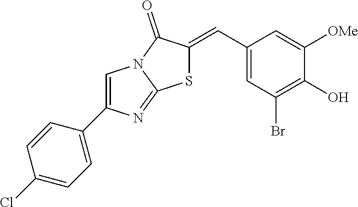 [SMILES: COc1cc(/C=c2\sc3nc(-c4ccc(Cl)cc4)cn3c2=O)cc(Br)c1O]

In analogy to the experimental procedure of example 1) 5-(4-chlorophenyl)-1H-imidazole-2-thiol was converted using 3-bromo-4-hydroxy-5-methoxybenzaldehyde instead of 4-hydroxy-3,5-dimethoxybenzaldehyde into the title compound (75 mg, 34%) which was obtained as a light brown solid. MS m/e: 465.1 [M+H]⁺

### EXAMPLE 8

### 6-(4-Chloro-phenyl)-2-[1-(3,4-dihydroxy-5-methoxy-phenyl)-meth-(Z)-ylidene]-imidazo[2,1-b]thiazol-3-one

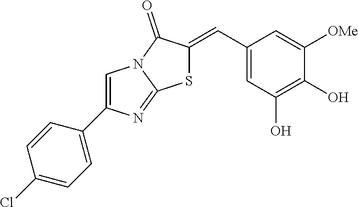 [SMILES: COc1cc(/C=c2\sc3nc(-c4ccc(Cl)cc4)cn3c2=O)cc(O)c1O]

In analogy to the experimental procedure of example 1) 5-(4-chlorophenyl)-1H-imidazole-2-thiol was converted using 3,4-dihydroxy-5-methoxybenzaldehyde instead of 4-hydroxy-3,5-dimethoxybenzaldehyde into the title compound (135 mg, 71%) which was obtained as a light brown solid. MS m/e: 401.0 [M+H]⁺

### EXAMPLE 9

### 2-[1-(3-Fluoro-4-hydroxy-5-methoxy-phenyl)-meth-(Z)-ylidene]-6-o-tolyl-imidazo[2,1-b]thiazol-3-one

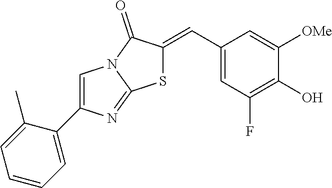 [SMILES: COc1cc(/C=c2\sc3nc(-c4ccccc4C)cn3c2=O)cc(F)c1O]

To a suspension of 4-o-tolyl-1H-imidazole-2(3H)-thione (100 mg, 526 μmol) and N,N-diisopropylethylamine (102 mg, 138 μl, 788 μmol) in dioxane (1.5 mL) was added at 10° C. dropwise chloroacetyl chloride (71.2 mg, 50.5 μl, 631 μmol). After stirring for 2 min 3-fluoro-4-hydroxy-5-methoxybenzaldehyde (93.9 mg, 552 μmol) was added and the reaction mixture stirred for 18 h at 110° C. After cooling to ambient temperature water (3 mL) was added and stirred for 15 min. The suspension was centrifuged and the upper layer was pipetted off and the solid was washed with water (2 mL). After the addition of ethanol (3 mL) it was stirred for 1 h at 80° C. It was centrifuged and the upper layer was pipetted off. The residue was washed twice with ethanol (2 mL) affording the title compound (65 mg, 32%) as a light brown solid. MS m/e: 383.2 [M+H]⁺

### EXAMPLE 10

### 2-[1-(3-Chloro-4-hydroxy-5-methoxy-phenyl)-meth-(Z)-ylidene]-6-o-tolyl-imidazo[2,1-b]thiazol-3-one

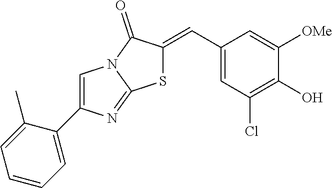 [SMILES: COc1cc(/C=c2\sc3nc(-c4ccccc4C)cn3c2=O)cc(Cl)c1O]

In analogy to the experimental procedure of example 9) 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (86 mg, 41%) which was obtained as a light brown solid. MS m/e: 399.1 [M+H]⁺

### EXAMPLE 11

### 2-[1-(4-hydroxy-3-methoxy-phenyl)-meth-(Z)-ylidene]-6-o-tolyl-imidazo[2,1-b]thiazol-3-one

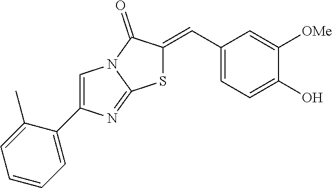 [SMILES: COc1cc(/C=c2\sc3nc(-c4ccccc4C)cn3c2=O)ccc1O]

In analogy to the experimental procedure of example 9) 4-o-tolyl-1H-imidazole-2(3H)-thione was converted using 4-hydroxy-3-methoxybenzaldehyde instead of 3-fluoro-4-hydroxy-5-methoxybenzaldehyde into the title compound (94 mg, 49%) which was obtained as a yellow solid. MS m/e: 365.1 [M+H]⁺

### EXAMPLE 12

### 2-[1-(4-Hydroxy-3-methoxy-phenyl)-meth-(Z)-ylidene]-6-(2-methyl-pyridin-4-yl)-imidazo[2,1-b]thiazol-3-one

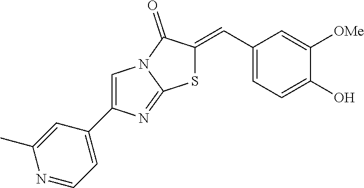 [SMILES: COc1cc(/C=c2\sc3nc(-c4ccnc(C)c4)cn3c2=O)ccc1O]

In analogy to the experimental procedure of example 9) 4-(2-methylpyridin-4-yl)-1H-imidazole-2(3H)-thione instead of 4-o-tolyl-1H-imidazole-2(3H)-thione was converted using 4-hydroxy-3-methoxybenzaldehyde instead of 3-fluoro-4-hydroxy-5-methoxybenzaldehyde into the title compound (22 mg, 8%) which was obtained as a light brown solid. MS m/e: 364.1 [M+H]⁺

### EXAMPLE 13

### 2-[1-(4-Hydroxy-3-methoxy-phenyl)-meth-(Z)-ylidene]-6-(2,6-dimethyl-pyridin-4-yl)-imidazo[2,1-b]thiazol-3-one

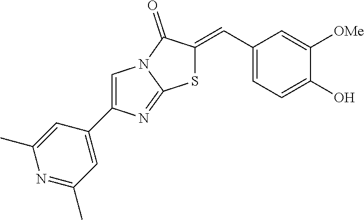 [SMILES: COc1cc(/C=c2\sc3nc(-c4cc(C)nc(C)c4)cn3c2=O)ccc1O]

In analogy to the experimental procedure of example 9) 4-(2,6-dimethylpyridin-4-yl)-1H-imidazole-2(3H)-thione instead of 4-o-tolyl-1H-imidazole-2(3H)-thione was converted using 4-hydroxy-3-methoxybenzaldehyde instead of 3-fluoro-4-hydroxy-5-methoxybenzaldehyde into the title compound (5 mg, 3%) which was obtained as a brown solid. MS m/e: 380.1 [M+H]⁺

### EXAMPLE 14

### 2-[1-(3-Chloro-4-hydroxy-5-methoxy-phenyl)-meth-(Z)-ylidene]-6-(4-methoxy-phenyl)-5-methyl-imidazo[2,1-b]thiazol-3-one

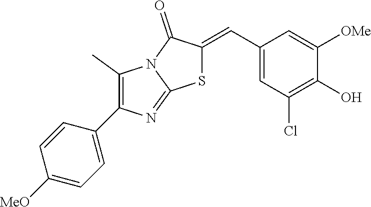 [SMILES: COc1ccc(-c2nc3s/c(=C\c4cc(Cl)c(O)c(OC)c4)c(=O)n3c2C)cc1]

In analogy to the experimental procedure of example 9) 4-(4-methoxyphenyl)-5-methyl-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (46 mg, 24%) which was obtained as a brown solid. MS m/e: 429.1 [M+H]⁺

### EXAMPLE 15

### 2-[1-(3-Fluoro-4-hydroxy-5-methoxy-phenyl)-meth-(Z)-ylidene]-6-(4-methoxy-phenyl)-5-methyl-imidazo[2,1-b]thiazol-3-one

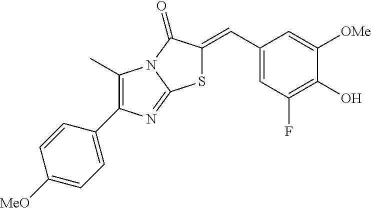 [SMILES: COc1ccc(-c2nc3s/c(=C\c4cc(F)c(O)c(OC)c4)c(=O)n3c2C)cc1]

In analogy to the experimental procedure of example 9) 4-(4-methoxyphenyl)-5-methyl-1H-imidazole-2(3H)-thione instead of 4-o-tolyl-1H-imidazole-2(3H)-thione was converted into the title compound (35 mg, 19%) which was obtained as a brown solid. MS m/e: 413.1 [M+H]⁺

### EXAMPLE 16

### 2-[1-(4-Hydroxy-3,5-dimethoxy-phenyl)-meth-(Z)-ylidene]-6-(2-methyl-pyridin-4-yl)-imidazo[2,1-b]thiazol-3-one

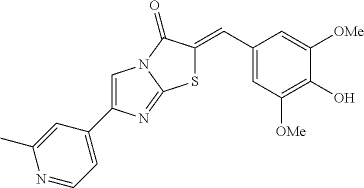 [SMILES: COc1cc(/C=c2\sc3nc(-c4ccnc(C)c4)cn3c2=O)cc(OC)c1O]

In analogy to the experimental procedure of example 9) 4-(2-methylpyridin-4-yl)-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (25 mg, 17%) which was obtained as an orange solid. MS m/e: 396.1 [M+H]⁺

### EXAMPLE 17

### 2-[1-(4-Hydroxy-3,5-dimethoxy-phenyl)-meth-(Z)-ylidene]-6-(2,6-dimethyl-pyridin-4-yl)-imidazo[2,1-b]thiazol-3-one

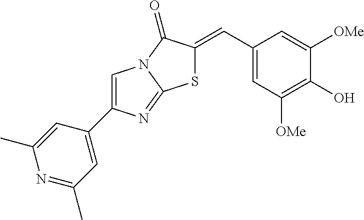 [SMILES: COc1cc(/C=c2\sc3nc(-c4cc(C)nc(C)c4)cn3c2=O)cc(OC)c1O]

In analogy to the experimental procedure of example 9) 4-(2,6-dimethylpyridin-4-yl)-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (14 mg, 7%) which was obtained as a red solid. MS m/e: 410.1 [M+H]⁺

### EXAMPLE 18

### 2-[1-(4-Hydroxy-3,5-dimethoxy-phenyl)-meth-(Z)-ylidene]-6-(3-trifluoromethoxy-phenyl)-imidazo[2,1-b]thiazol-3-one

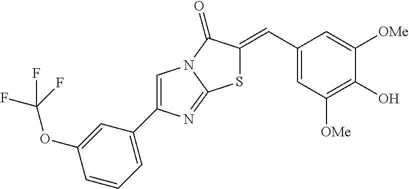 [SMILES: COc1cc(/C=c2\sc3nc(-c4cccc(OC(F)(F)F)c4)cn3c2=O)cc(OC)c1O]

In analogy to the experimental procedure of example 9) 4-(3-(trifluoromethoxy)phenyl)-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (35 mg, 20%) which was obtained as a yellow solid. MS m/e: 465.1 [M+H]⁺

### EXAMPLE 19

### 2-[1-(4-Hydroxy-3,5-dimethoxy-phenyl)-meth-(Z)-ylidene]-6-(3-trifluoromethyl-phenyl)-imidazo[2,1-b]thiazol-3-one

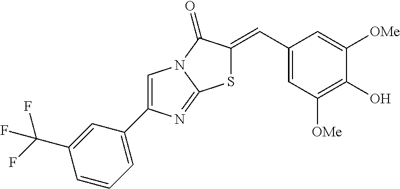 [SMILES: COc1cc(/C=c2\sc3nc(-c4cccc(C(F)(F)F)c4)cn3c2=O)cc(OC)c1O]

In analogy to the experimental procedure of example 9) 4-(3-(trifluoromethyl)phenyl)-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (16 mg, 9%) which was obtained as an orange solid. MS m/e: 449.1 [M+H]⁺

### EXAMPLE 20

### 2-[1-(4-Hydroxy-3,5-dimethoxy-phenyl)-meth-(Z)-ylidene]-6-m-tolyl-imidazo[2,1-b]thiazol-3-one

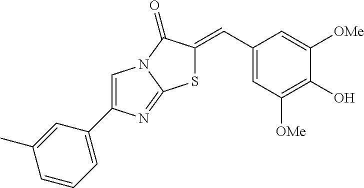 [SMILES: COc1cc(/C=c2\sc3nc(-c4cccc(C)c4)cn3c2=O)cc(OC)c1O]

In analogy to the experimental procedure of example 9) 4-m-tolyl-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (50 mg, 28%) which was obtained as a yellow solid. MS m/e: 395.1 [M+H]⁺

### EXAMPLE 21

### 6-(3-Chloro-phenyl)-2-[1-(4-hydroxy-3,5-dimethoxy-phenyl)-meth-(Z)-ylidene]-imidazo[2,1-b]thiazol-3-one

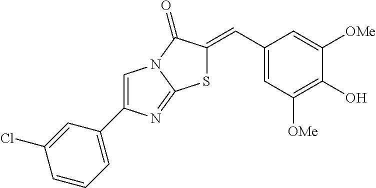 [SMILES: COc1cc(/C=c2\sc3nc(-c4cccc(Cl)c4)cn3c2=O)cc(OC)c1O]

In analogy to the experimental procedure of example 9) 4-(3-chlorophenyl)-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (60 mg, 31%) which was obtained as a yellow solid. MS m/e: 415.1 [M+H]⁺

### EXAMPLE 22

### 2-[1-(3-Chloro-4-hydroxy-5-methoxy-phenyl)-meth-(Z)-ylidene]-6-(3-trifluoromethoxy-phenyl)-imidazo[2,1-b]thiazol-3-one

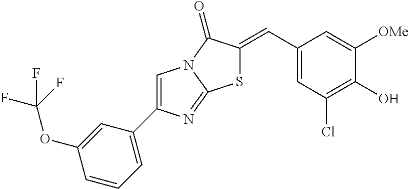 [SMILES: COc1cc(/C=c2\sc3nc(-c4cccc(OC(F)(F)F)c4)cn3c2=O)cc(Cl)c1O]

In analogy to the experimental procedure of example 9) 4-(3-(trifluoromethoxy)phenyl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (19 mg, 11%) which was obtained as a orange solid. MS m/e: 469.1 [M+H]⁺

### EXAMPLE 23

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(2-fluoro-ethoxy)-ethyl]-N-methyl-benzamide

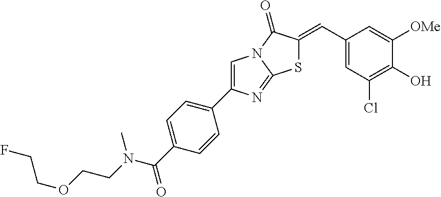 [SMILES: COc1cc(/C=c2\sc3nc(-c4ccc(C(=O)N(C)CCOCCF)cc4)cn3c2=O)cc(Cl)c1O]

In analogy to the experimental procedure of example 105) of (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 2-(2-fluoroethoxy)-N-methylethanamine hydrochloride hydrochloride instead of 2-fluoroethylamine hydrochloride into the title compound (33 mg, 24%) which was obtained as a yellow solid. MS m/e: 532.1 [M+H]⁺

### EXAMPLE 24

### 2-[1-(3-Chloro-4-hydroxy-5-methoxy-phenyl)-meth-(Z)-ylidene]-6-(3-trifluoromethyl-phenyl)-imidazo[2,1-b]thiazol-3-one

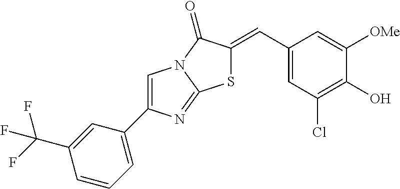 [SMILES: COc1cc(/C=c2\sc3nc(-c4cccc(C(F)(F)F)c4)cn3c2=O)cc(Cl)c1O]

In analogy to the experimental procedure of example 9) 4-(3-(trifluoromethyl)phenyl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (29 mg, 16%) which was obtained as a yellow solid. MS m/e: 453.1 [M+H]⁺

### EXAMPLE 25

### 6-(3-Chloro-phenyl)-2-[1-(3-chloro-4-hydroxy-5-methoxy-phenyl)-meth-(Z)-ylidene]-imidazo[2,1-b]thiazol-3-one

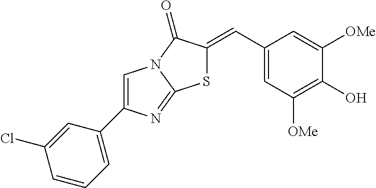 [SMILES: COc1cc(/C=c2\sc3nc(-c4cccc(Cl)c4)cn3c2=O)cc(OC)c1O]

In analogy to the experimental procedure of example 9) 4-(3-chlorophenyl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (61 mg, 31%) which was obtained as a yellow solid. MS m/e: 419.1 [M+H]⁺

### EXAMPLE 26

### 6-(4-Chloro-phenyl)-2-[1-(3-ethoxy-4-hydroxy-5-methoxy-phenyl)-meth-(Z)-ylidene]-imidazo[2,1-b]thiazol-3-one

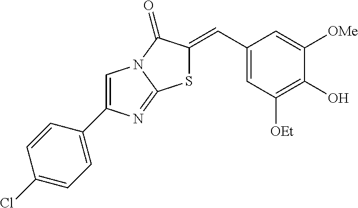 [SMILES: CCOc1cc(/C=c2\sc3nc(-c4ccc(Cl)cc4)cn3c2=O)cc(OC)c1O]

In analogy to the experimental procedure of example 9) 4-(4-chlorophenyl)-1H-imidazole-2(3H)-thione instead of 4-o-tolyl-1H-imidazole-2(3H)-thione was converted using 3-ethoxy-4-hydroxy-5-methoxybenzaldehyde instead of 3-fluoro-4-hydroxy-5-methoxybenzaldehyde into the title compound (58 mg, 29%) which was obtained as a yellow solid. MS m/e: 429.1 [M+H]⁺

### EXAMPLE 27

### 6-(4-Chloro-phenyl)-2-[1-(3-(2-fluoro-ethoxy)-4-hydroxy-5-methoxy-phenyl)-meth-(Z)-ylidene]-imidazo[2,1-b]thiazol-3-one

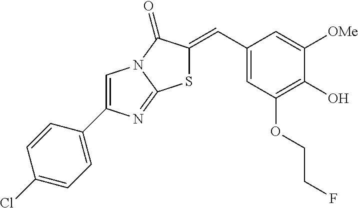 [SMILES: COc1cc(/C=c2\sc3nc(-c4ccc(Cl)cc4)cn3c2=O)cc(OCCF)c1O]

In analogy to the experimental procedure of example 9) 4-(4-chlorophenyl)-1H-imidazole-2(3H)-thione instead of 4-o-tolyl-1H-imidazole-2(3H)-thione was converted using 3-(2-fluoro-ethoxy)-4-hydroxy-5-methoxybenzaldehyde instead of 3-fluoro-4-hydroxy-5-methoxybenzaldehyde into the title compound (71 mg, 34%) which was obtained as a yellow solid. MS m/e: 447.1 [M+H]⁺

### EXAMPLE 28

### 2-[1-(3-Chloro-4-hydroxy-5-methoxy-phenyl)-meth-(Z)-ylidene]-6-(3-fluoro-4-trifluoromethyl-phenyl)-imidazo[2,1-b]thiazol-3-one

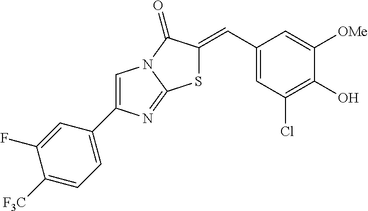 [SMILES: COc1cc(/C=c2\sc3nc(-c4ccc(C(F)(F)F)c(F)c4)cn3c2=O)cc(Cl)c1O]

In analogy to the experimental procedure of example 9) 4-(3-fluoro-4-trifluoromethyl-phenyl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (62 mg, 35%) which was obtained as an orange solid. MS m/e: 469.1 [M+H]⁺

### EXAMPLE 29

### 2-[1-(3-Hydroxy-3,5-dimethoxy-phenyl)-meth-(Z)-ylidene]-6-(3-fluoro-4-trifluoromethyl-phenyl)-imidazo[2,1-b]thiazol-3-one

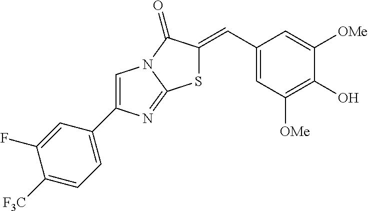 [SMILES: COc1cc(/C=c2\sc3nc(-c4ccc(C(F)(F)F)c(F)c4)cn3c2=O)cc(OC)c1O]

In analogy to the experimental procedure of example 9) 4-(3-fluoro-4-trifluoromethyl-phenyl)-1H-imidazole-2(3H)-thione instead of 4-o-tolyl-1H-imidazole-2(3H)-thione was converted using 4-hydroxy-3,5-dimethoxybenzaldehyde instead of 3-fluoro-4-hydroxy-5-methoxybenzaldehyde into the title compound (50 mg, 28%) which was obtained as a yellow solid. MS m/e: 467.1 [M+H]⁺

### EXAMPLE 30

### 6-(4-Chloro-phenyl)-2-[1-(4-hydroxy-3-isopropoxy-5-methoxy-phenyl)-meth-(Z)-ylidene]-imidazo[2,1-b]thiazol-3-one

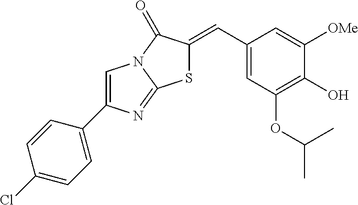 [SMILES: COc1cc(/C=c2\sc3nc(-c4ccc(Cl)cc4)cn3c2=O)cc(OC(C)C)c1O]

In analogy to the experimental procedure of example 9) 4-(4-chloro-phenyl)-1H-imidazole-2(3H)-thione instead of 4-o-tolyl-1H-imidazole-2(3H)-thione was converted using 4-hydroxy-3-isopropoxy-5-methoxybenzaldehyde instead of 3-fluoro-4-hydroxy-5-methoxybenzaldehyde into the title compound (37 mg, 18%) which was obtained as a yellow solid. MS m/e: 443.1 [M+H]⁺

### EXAMPLE 31

### 6-(4-Chloro-phenyl)-2-[1-(4-hydroxy-3-methoxy-5-propoxy-phenyl)-meth-(Z)-ylidene]-imidazo[2,1-b]thiazol-3-one

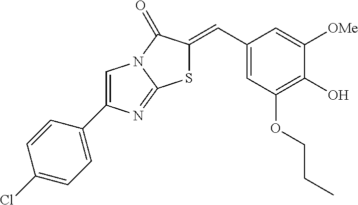 [SMILES: CCCOc1cc(/C=c2\sc3nc(-c4ccc(Cl)cc4)cn3c2=O)cc(OC)c1O]

In analogy to the experimental procedure of example 9) 4-(4-chloro-phenyl)-1H-imidazole-2(3H)-thione instead of 4-o-tolyl-1H-imidazole-2(3H)-thione was converted using 4-hydroxy-3-methoxy-5-propoxybenzaldehyde instead of 3-fluoro-4-hydroxy-5-methoxybenzaldehyde into the title compound (45 mg, 21%) which was obtained as a yellow solid. MS m/e: 443.1 [M+H]⁺

### EXAMPLE 32

### 6-(4-Chloro-phenyl)-2-[1-(4-hydroxy-3-methoxy-5-methyl-phenyl)-meth-(Z)-ylidene]-imidazo[2,1-b]thiazol-3-one

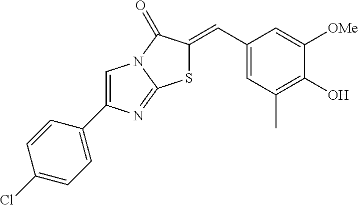 [SMILES: COc1cc(/C=c2\sc3nc(-c4ccc(Cl)cc4)cn3c2=O)cc(C)c1O]

In analogy to the experimental procedure of example 1) 5-(4-chlorophenyl)-1H-imidazole-2-thiol instead of 5-(pyridin-4-yl)-1H-imidazole-2-thiol was converted using 4-hydroxy-3-methoxy-5-methylbenzaldehyde instead of 4-hydroxy-3,5-dimethoxybenzaldehyde into the title compound (31 mg, 26%) which was obtained as a yellow solid. MS m/e: 399.1 [M+H]⁺

### EXAMPLE 33

### 2-[1-(4-Hydroxy-3-methoxy-5-propoxy-phenyl)-meth-(Z)-ylidene]-6-(3-trifluoromethoxy-phenyl)-imidazo[2,1-b]thiazol-3-one

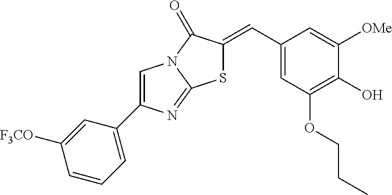 [SMILES: CCCOc1cc(/C=c2\sc3nc(-c4cccc(OC(F)(F)F)c4)cn3c2=O)cc(OC)c1O]

In analogy to the experimental procedure of example 1) 5-(3-trifluoromethoxy-phenyl)-1H-imidazole-2-thiol instead of 5-(pyridin-4-yl)-1H-imidazole-2-thiol was converted using 4-hydroxy-3-methoxy-5-propoxybenzaldehyde instead of 4-hydroxy-3,5-dimethoxybenzaldehyde into the title compound (65 mg, 34%) which was obtained as a yellow solid. MS m/e: 493.1 [M+H]⁺

### EXAMPLE 34

### 2-[1-(4-Hydroxy-3-methoxy-5-isopropoxy-phenyl)-meth-(Z)-ylidene]-6-(3-trifluoromethoxy-phenyl)-imidazo[2,1-b]thiazol-3-one

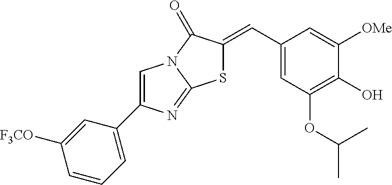 [SMILES: COc1cc(/C=c2\sc3nc(-c4cccc(OC(F)(F)F)c4)cn3c2=O)cc(OC(C)C)c1O]

In analogy to the experimental procedure of example 1) 5-(3-trifluoromethoxy-phenyl)-1H-imidazole-2-thiol instead of 5-(pyridin-4-yl)-1H-imidazole-2-thiol was converted using 4-hydroxy-3-methoxy-5-isopropoxybenzaldehyde instead of 4-hydroxy-3,5-dimethoxybenzaldehyde into the title compound (29 mg, 15%) which was obtained as a yellow solid. MS m/e: 493.1 [M+H]⁺

### EXAMPLE 35

### 2-[1-(3-Fluoro-4-hydroxy-5-methoxy-phenyl)-meth-(Z)-ylidene]-6-(3-trifluoromethoxy-phenyl)-imidazo[2,1-b]thiazol-3-one

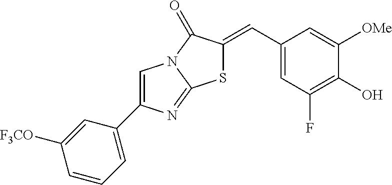 [SMILES: COc1cc(/C=c2\sc3nc(-c4cccc(OC(F)(F)F)c4)cn3c2=O)cc(F)c1O]

In analogy to the experimental procedure of example 1) 5-(3-trifluoromethoxy-phenyl)-1H-imidazole-2-thiol instead of 5-(pyridin-4-yl)-1H-imidazole-2-thiol was converted using 3-fluoro-4-hydroxy-5-methoxybenz aldehyde instead of 4-hydroxy-3,5-dimethoxybenzaldehyde into the title compound (39 mg, 22%) which was obtained as a yellow solid. MS m/e: 453.1 [M+H]⁺

### EXAMPLE 36

### 2-[1-(4-Hydroxy-3-methoxy-phenyl)-meth-(Z)-ylidene]-6-(3-trifluoromethoxy-phenyl)-imidazo[2,1-b]thiazol-3-one

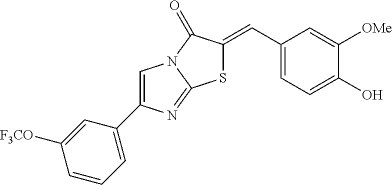 [SMILES: COc1cc(/C=c2\sc3nc(-c4cccc(OC(F)(F)F)c4)cn3c2=O)ccc1O]

In analogy to the experimental procedure of example 1) 5-(3-trifluoromethoxy-phenyl)-1H-imidazole-2-thiol instead of 5-(pyridin-4-yl)-1H-imidazole-2-thiol was converted using 4-hydroxy-3-methoxybenzaldehyde instead of 4-hydroxy-3,5-dimethoxybenzaldehyde into the title compound (35 mg, 21%) which was obtained as a yellow solid. MS m/e: 435.1 [M+H]⁺

### EXAMPLE 37

### 2-[1-(3-Bromo-4-hydroxy-5-methoxy-phenyl)-meth-(Z)-ylidene]-6-(3-trifluoromethoxy-phenyl)-imidazo[2,1-b]thiazol-3-one

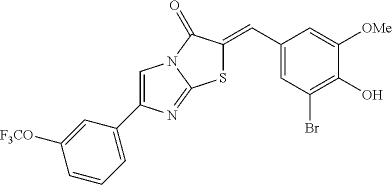 [SMILES: COc1cc(/C=c2\sc3nc(-c4cccc(OC(F)(F)F)c4)cn3c2=O)cc(Br)c1O]

In analogy to the experimental procedure of example 1) 5-(3-trifluoromethoxy-phenyl)-1H-imidazole-2-thiol instead of 5-(pyridin-4-yl)-1H-imidazole-2-thiol was converted using 3-bromo-4-hydroxy-5-methoxybenzaldehyde instead of 4-hydroxy-3,5-dimethoxybenzaldehyde into the title compound (62 mg, 31%) which was obtained as a yellow solid. MS m/e: 514.9 [M+H]⁺

### EXAMPLE 38

### 3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzonitrile

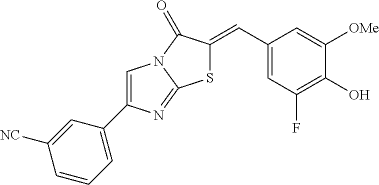 [SMILES: COc1cc(/C=c2\sc3nc(-c4cccc(C#N)c4)cn3c2=O)cc(F)c1O]

In analogy to the experimental procedure of example 1) 3-(2-thioxo-2,3-dihydro-1H-imidazol-4-yl)benzonitrile instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (62 mg, 32%) which was obtained as a yellow solid. MS m/e: 394.1 [M+H]⁺

### EXAMPLE 39

### 2-[1-(3-fluoro-4-hydroxy-5-methoxy-phenyl)-meth-(Z)-ylidene]-6-(3-chloro-phenyl)-imidazo[2,1-b]thiazol-3-one

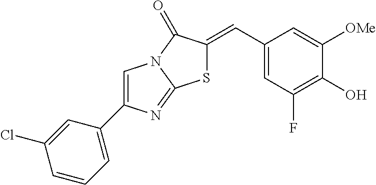 [SMILES: COc1cc(/C=c2\sc3nc(-c4cccc(Cl)c4)cn3c2=O)cc(F)c1O]

In analogy to the experimental procedure of example 1) 4-(3-chlorophenyl)-1H-imidazole-2(3H)-thione instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (58 mg, 30%) which was obtained as a yellow solid. MS m/e: 403.0 [M+H]⁺

### EXAMPLE 40

### 2-[1-(3-Ethyl-4-hydroxy-5-methoxy-phenyl)-meth-(Z)-ylidene]-6-(3-trifluoromethoxy-phenyl)-imidazo[2,1-b]thiazol-3-one

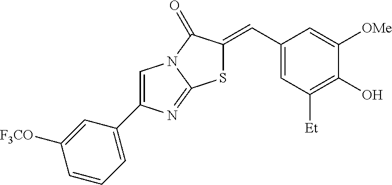 [SMILES: CCc1cc(/C=c2\sc3nc(-c4cccc(OC(F)(F)F)c4)cn3c2=O)cc(OC)c1O]

In analogy to the experimental procedure of example 1) 5-(3-trifluoromethoxy-phenyl)-1H-imidazole-2-thiol instead of 5-(pyridin-4-yl)-1H-imidazole-2-thiol was converted using 3-ethyl-4-hydroxy-5-methoxybenz aldehyde instead of 4-hydroxy-3,5-dimethoxybenz aldehyde into the title compound (64 mg, 36%) which was obtained as a yellow solid. MS m/e: 463.1 [M+H]⁺

### EXAMPLE 41

### 2-[1-(3-fluoro-4-hydroxy-5-methoxy-phenyl)-meth-(Z)-ylidene]-6-(3-(trifluoromethyl)-phenyl)-imidazo[2,1-b]thiazol-3-one

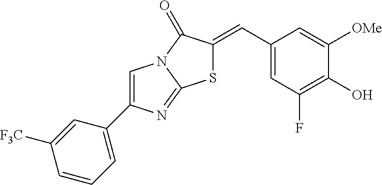 [SMILES: COc1cc(/C=c2\sc3nc(-c4cccc(C(F)(F)F)c4)cn3c2=O)cc(F)c1O]

In analogy to the experimental procedure of example 1) 4-(3-(trifluoromethyl)phenyl)-1H-imidazole-2(3H)-thione instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (56 mg, 31%) which was obtained as a yellow solid. MS m/e: 437.1 [M+H]⁺

### EXAMPLE 42

### 2-[1-(3-fluoro-4-hydroxy-5-methoxy-phenyl)-meth-(Z)-ylidene]-6-(4-fluoro-3-(trifluoromethyl)-phenyl)-imidazo[2,1-b]thiazol-3-one

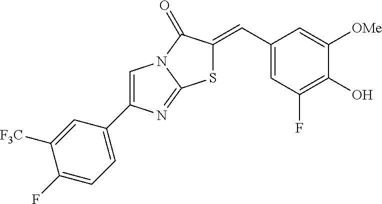 [SMILES: COc1cc(/C=c2\sc3nc(-c4ccc(F)c(C(F)(F)F)c4)cn3c2=O)cc(F)c1O]

In analogy to the experimental procedure of example 1) 4-(4-fluoro-3-(trifluoromethyl)phenyl)-1H-imidazole-2(3H)-thione instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (45 mg, 19%) which was obtained as a yellow solid. MS m/e: 455.1 [M+H]⁺

### EXAMPLE 43

### 2-[1-(3-fluoro-4-hydroxy-5-methoxy-phenyl)-meth-(Z)-ylidene]-6-phenyl-imidazo[2,1-b]thiazol-3-one

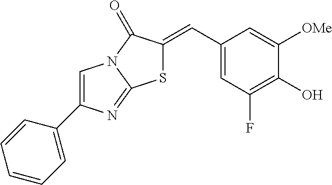 [SMILES: COc1cc(/C=c2\sc3nc(-c4ccccc4)cn3c2=O)cc(F)c1O]

To a solution of acetophenone (19.7 mg, 19.1 μl, 164 μmol) in acetonitrile (1 mL) was added under an atmosphere of nitrogen [hydroxy(tosyloxy)iodo]benzene (64.3 mg, 164 μmol). The reaction mixture was stirred at 80° C. for 3 h. The resulting solution was concentrated in vacuo and a suspension of (Z)-2-amino-5-(3-fluoro-4-hydroxy-5-methoxybenzylidene)thiazol-4(5H)-one (44 mg, 164 μmol) in ethanol (3 mL) was added. The yellow suspension was stirred at 80° C. for 3 h. It was diluted with tetrahydrofurane (2 mL) and the resulting solution was stirred at 80° C. for 18 h. The reaction mixture was furthermore irradiated in the microwave at 140° C. for 60 min. At ambient temperature the solution was diluted with water (10 mL), centrifuged and the upper layer was pipetted off. Purification of the residue by flash chromatography (SiO₂, eluent: Heptane/dichloromethane/methanol=20:80:0 to 0:95:5) afforded the title compound (4 mg, 8%) as a yellow solid. MS m/e: 369.07 [M+H]⁺

### EXAMPLE 44

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzonitrile

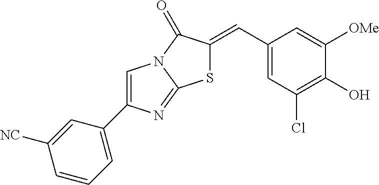 [SMILES: COc1cc(/C=c2\sc3nc(-c4cccc(C#N)c4)cn3c2=O)cc(Cl)c1O]

In analogy to the experimental procedure of example 1) 3-(2-thioxo-2,3-dihydro-1H-imidazol-4-yl)benzonitrile instead of 5-(pyridin-4-yl)-1H-imidazole-2-thiol was converted using 3-chloro-4-hydroxy-5-methoxybenzaldehyde instead of 4-hydroxy-3,5-dimethoxybenzaldehyde into the title compound (34 mg, 17%) which was obtained as a yellow solid. MS m/e: 410.0 [M+H]⁺

### EXAMPLE 45

### 3-{2-[1-(3-Bromo-4-hydroxy-5-methoxy-phenyl)-meth-(Z)-ylidene]-3-oxo-2,3-dihydro-imidazo[2,1-b]thiazol-6-yl}-benzonitrile

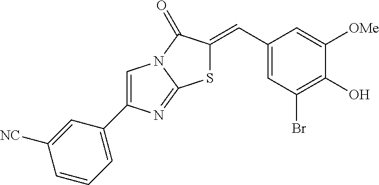 [SMILES: COc1cc(/C=c2\sc3nc(-c4cccc(C#N)c4)cn3c2=O)cc(Br)c1O]

In analogy to the experimental procedure of example 1) 3-(2-thioxo-2,3-dihydro-1H-imidazol-4-yl)benzonitrile instead of 5-(pyridin-4-yl)-1H-imidazole-2-thiol was converted using 3-bromo-4-hydroxy-5-methoxybenzaldehyde instead of 4-hydroxy-3,5-dimethoxybenzaldehyde into the title compound (80 mg, 35%) which was obtained as a yellow solid. MS m/e: 455.9 [M+H]⁺

### EXAMPLE 46

### 2-[1-(3-bromo-4-hydroxy-5-methoxy-phenyl)-meth-(Z)-ylidene]-6-(4-fluoro-3-(trifluoromethyl)-phenyl)-imidazo[2,1-b]thiazol-3-one

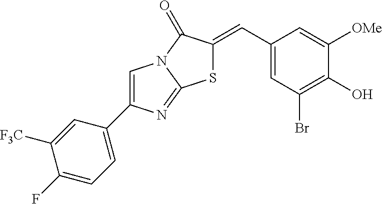 [SMILES: COc1cc(/C=c2\sc3nc(-c4ccc(F)c(C(F)(F)F)c4)cn3c2=O)cc(Br)c1O]

In analogy to the experimental procedure of example 1) 4-(4-fluoro-3-(trifluoromethyl)phenyl)-1H-imidazole-2(3H)-thione instead of 5-(pyridin-4-yl)-1H-imidazole-2-thiol was converted using 3-bromo-4-hydroxy-5-methoxybenzaldehyde instead of 4-hydroxy-3,5-dimethoxybenzaldehyde into the title compound (57 mg, 29%) which was obtained as a yellow solid. MS m/e: 516.9 [M+H]⁺

### EXAMPLE 47

### 2-[1-(3-Fluoro-4-hydroxy-5-methoxy-phenyl)-meth-(Z)-ylidene]-6-(3-methoxy-phenyl)-imidazo[2,1-b]thiazol-3-one

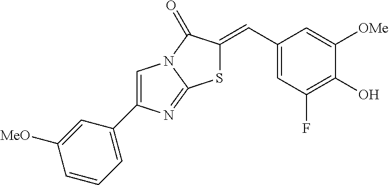 [SMILES: COc1cccc(-c2cn3c(=O)/c(=C/c4cc(F)c(O)c(OC)c4)sc3n2)c1]

In analogy to the experimental procedure of example 43) 1-(3-methoxy-phenyl)-ethanone instead of acetophenone was converted into the title compound (17 mg, 11%) which was obtained as a yellow solid. MS m/e: 399.1 [M+H]⁺

### EXAMPLE 48

### 2-[1-(3-Fluoro-4-hydroxy-5-methoxy-phenyl)-meth-(Z)-ylidene]-6-(6-methoxy-pyridin-2-yl)-imidazo[2,1-b]thiazol-3-one

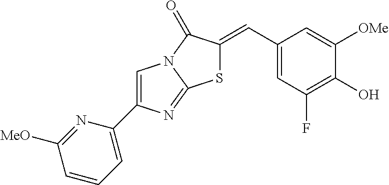 [SMILES: COc1cccc(-c2cn3c(=O)/c(=C/c4cc(F)c(O)c(OC)c4)sc3n2)n1]

In analogy to the experimental procedure of example 43) 1-(6-methoxypyridin-2-yl)ethanone instead of acetophenone was converted into the title compound (5 mg, 3%) which was obtained as a yellow solid. MS m/e: 400.1 [M+H]⁺

### EXAMPLE 49

### 2-[1-(3-Chloro-4-hydroxy-5-methoxy-phenyl)-meth-(Z)-ylidene]-6-(3,5-difluoro-phenyl)-imidazo[2,1-b]thiazol-3-one

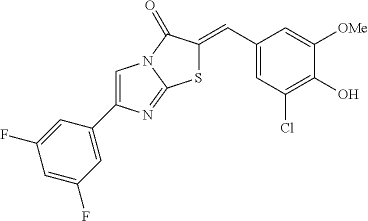 [SMILES: COc1cc(/C=c2\sc3nc(-c4cc(F)cc(F)c4)cn3c2=O)cc(Cl)c1O]

In analogy to the experimental procedure of example 43) 1-(3,5-difluorophenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (24 mg, 16%) which was obtained as a yellow solid. MS m/e: 421.1 [M+H]⁺

### EXAMPLE 50

### 3-{2-[1-(4-Hydroxy-3,5-dimethoxy-phenyl)-meth-(Z)-ylidene]-3-oxo-2,3-dihydro-imidazo[2,1-b]thiazol-6-yl}-benzonitrile

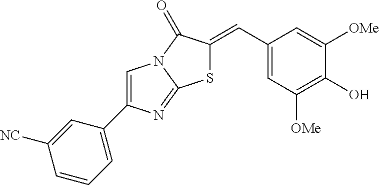 [SMILES: COc1cc(/C=c2\sc3nc(-c4cccc(C#N)c4)cn3c2=O)cc(OC)c1O]

In analogy to the experimental procedure of example 1) 3-(2-thioxo-2,3-dihydro-1H-imidazol-4-yl)benzonitrile instead of 5-(pyridin-4-yl)-1H-imidazole-2-thiol was converted into the title compound (53 mg, 26%) which was obtained as a yellow solid. MS m/e: 406.1 [M+H]⁺

### EXAMPLE 51

### 3-{2-[1-(4-Hydroxy-3-methoxy-5-isopropoxyphenyl)-meth-(Z)-ylidene]-3-oxo-2,3-dihydro-imidazo[2,1-b]thiazol-6-yl}-benzonitrile

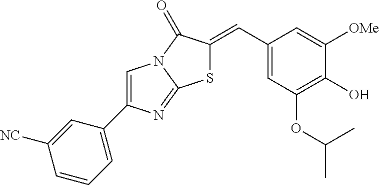 [SMILES: COc1cc(/C=c2\sc3nc(-c4cccc(C#N)c4)cn3c2=O)cc(OC(C)C)c1O]

In analogy to the experimental procedure of example 1) 3-(2-thioxo-2,3-dihydro-1H-imidazol-4-yl)benzonitrile instead of 5-(pyridin-4-yl)-1H-imidazole-2-thiol was converted using 4-hydroxy-3-methoxy-5-isopropoxy-benzaldehyde instead of 4-hydroxy-3,5-dimethoxybenzaldehyde into the title compound (27 mg, 13%) which was obtained as a yellow solid. MS m/e: 434.1 [M+H]⁺

### EXAMPLE 52

### 2-[1-(3-Chloro-4-hydroxy-5-methoxy-phenyl)-meth-(Z)-ylidene]-6-m-tolyl-imidazo[2,1-b]thiazol-3-one

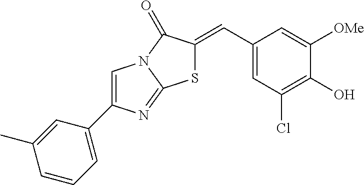 [SMILES: COc1cc(/C=c2\sc3nc(-c4cccc(C)c4)cn3c2=O)cc(Cl)c1O]

In analogy to the experimental procedure of example 43) 1-m-tolylethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (16 mg, 11%) which was obtained as a yellow solid. MS m/e: 399.1 [M+H]⁺

### EXAMPLE 53

### 2-[1-(3-Chloro-4-hydroxy-5-methoxy-phenyl)-meth-(Z)-ylidene]-6-(2-fluoro-phenyl)-imidazo[2,1-b]thiazol-3-one

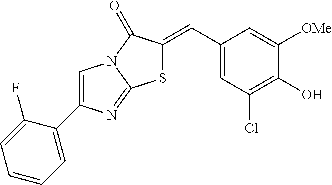 [SMILES: COc1cc(/C=c2\sc3nc(-c4ccccc4F)cn3c2=O)cc(Cl)c1O]

In analogy to the experimental procedure of example 43) 1-(2-fluorophenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (11 mg, 8%) which was obtained as a yellow solid. MS m/e: 403.0 [M+H]⁺

### EXAMPLE 54

### 2-[1-(3-Chloro-4-hydroxy-5-methoxy-phenyl)-meth-(Z)-ylidene]-6-(3-ethyl-phenyl)-imidazo[2,1-b]thiazol-3-one

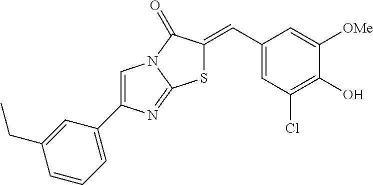 [SMILES: CCc1cccc(-c2cn3c(=O)/c(=C/c4cc(Cl)c(O)c(OC)c4)sc3n2)c1]

In analogy to the experimental procedure of example 43) 1-(3-ethylphenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (17 mg, 12%) which was obtained as a yellow solid. MS m/e: 412.1 [M+H]⁺

### EXAMPLE 55

### 2-[1-(3-Chloro-4-hydroxy-5-methoxy-phenyl)-meth-(Z)-ylidene]-6-(2-methoxy-phenyl)-imidazo[2,1-b]thiazol-3-one

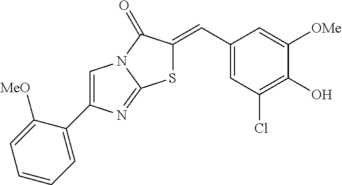 [SMILES: COc1ccccc1-c1cn2c(=O)/c(=C/c3cc(Cl)c(O)c(OC)c3)sc2n1]

In analogy to the experimental procedure of example 43) 1-(2-methoxyphenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (3 mg, 2%) which was obtained as a yellow solid. MS m/e: 415.1 [M+H]⁺

### EXAMPLE 56

### 2-[1-(3-Chloro-4-hydroxy-5-methoxy-phenyl)-meth-(Z)-ylidene]-6-(2,5-difluoro-phenyl)-imidazo[2,1-b]thiazol-3-one

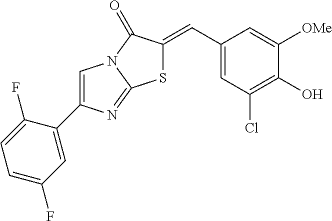 [SMILES: COc1cc(/C=c2\sc3nc(-c4cc(F)ccc4F)cn3c2=O)cc(Cl)c1O]

In analogy to the experimental procedure of example 43) 1-(2,5-difluorophenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (5 mg, 4%) which was obtained as a yellow solid. MS m/e: 421.0 [M+H]⁺

### EXAMPLE 57

### 2-[1-(3-Chloro-4-hydroxy-5-methoxy-phenyl)-meth-(Z)-ylidene]-6-(3-fluoro-phenyl)-imidazo[2,1-b]thiazol-3-one

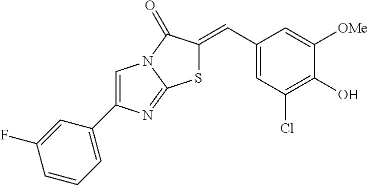 [SMILES: COc1cc(/C=c2\sc3nc(-c4cccc(F)c4)cn3c2=O)cc(Cl)c1O]

In analogy to the experimental procedure of example 1) 4-(3-fluorophenyl)-1H-imidazole-2(3H)-thione instead of 5-(pyridin-4-yl)-1H-imidazole-2-thiol was converted using 3-chloro-4-hydroxy-5-methoxybenzaldehyde instead of 4-hydroxy-3,5-dimethoxybenzaldehyde into the title compound (58 mg, 28%) which was obtained as a yellow solid. MS m/e: 403.1 [M+H]⁺

### EXAMPLE 58

### 2-[1-(3-Fluoro-4-hydroxy-5-methoxy-phenyl)-meth-(Z)-ylidene]-6-(3-fluoro-phenyl)-imidazo[2,1-b]thiazol-3-one

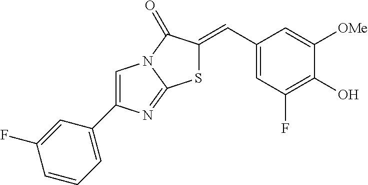 [SMILES: COc1cc(/C=c2\sc3nc(-c4cccc(F)c4)cn3c2=O)cc(F)c1O]

In analogy to the experimental procedure of example 1) 4-(3-fluorophenyl)-1H-imidazole-2(3H)-thione instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (54 mg, 27%) which was obtained as a yellow solid. MS m/e: 387.1 [M+H]⁺

### EXAMPLE 59

### 2-[1-(4-Hydroxy-3,5-dimethoxy-phenyl)-meth-(Z)-ylidene]-6-(3-fluoro-phenyl)-imidazo[2,1-b]thiazol-3-one

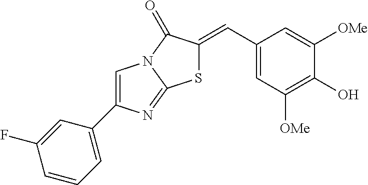 [SMILES: COc1cc(/C=c2\sc3nc(-c4cccc(F)c4)cn3c2=O)cc(OC)c1O]

In analogy to the experimental procedure of example 1) 4-(3-fluorophenyl)-1H-imidazole-2(3H)-thione instead of 5-(pyridin-4-yl)-1H-imidazole-2-thiol was converted into the title compound (26 mg, 13%) which was obtained as a yellow solid. MS m/e: 380.1 [M+H]⁺

### EXAMPLE 60

### 2-[1-(3-Chloro-4-hydroxy-5-methoxy-phenyl)-meth-(Z)-ylidene]-6-phenyl-imidazo[2,1-b]thiazol-3-one

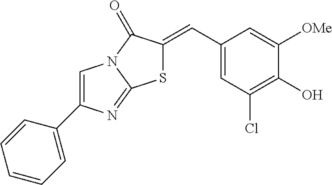 [SMILES: COc1cc(/C=c2\sc3nc(-c4ccccc4)cn3c2=O)cc(Cl)c1O]

In analogy to the experimental procedure of example 43) acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (17 mg, 13%) which was obtained as a yellow solid. MS m/e: 385.0 [M+H]⁺

### EXAMPLE 61

### 2-[1-(3-Chloro-4-hydroxy-5-methoxy-phenyl)-meth-(Z)-ylidene]-6-(3-methoxy-phenyl)-imidazo[2,1-b]thiazol-3-one

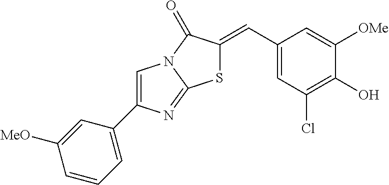 [SMILES: COc1cccc(-c2cn3c(=O)/c(=C/c4cc(Cl)c(O)c(OC)c4)sc3n2)c1]

In analogy to the experimental procedure of example 43) 1-(3-methoxyphenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (20 mg, 14%) which was obtained as a yellow solid. MS m/e: 415.1 [M+H]⁺

### EXAMPLE 62

### 2-[1-(3-Chloro-4-hydroxy-5-methoxy-phenyl)-meth-(Z)-ylidene]-6-(3-(difluoromethoxy)-phenyl)-imidazo[2,1-b]thiazol-3-one

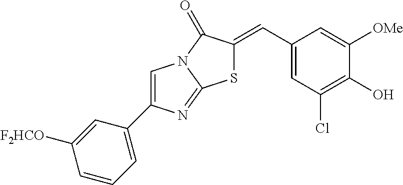 [SMILES: COc1cc(/C=c2\sc3nc(-c4cccc(OC(F)F)c4)cn3c2=O)cc(Cl)c1O]

In analogy to the experimental procedure of example 43) 1-(3-(difluoro)methoxyphenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (32 mg, 20%) which was obtained as a yellow solid. MS m/e: 451.0 [M+H]⁺

### EXAMPLE 63

### 2-[1-(3-Chloro-4-hydroxy-5-methoxy-phenyl)-meth-(Z)-ylidene]-6-(2,6-dimethyl-pyridin-4-yl)-imidazo[2,1-b]thiazol-3-one

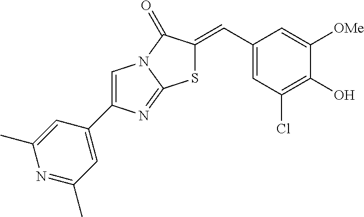 [SMILES: COc1cc(/C=c2\sc3nc(-c4cc(C)nc(C)c4)cn3c2=O)cc(Cl)c1O]

In analogy to the experimental procedure of example 9) 4-(2,6-dimethylpyridin-4-yl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (172 mg, 57%) which was obtained as a brown solid. MS m/e: 414.1 [M+H]⁺

### EXAMPLE 64

### 2-[1-(3-Fluoro-4-hydroxy-5-methoxy-phenyl)-meth-(Z)-ylidene]-6-(2,6-dimethyl-pyridin-4-yl)-imidazo[2,1-b]thiazol-3-one

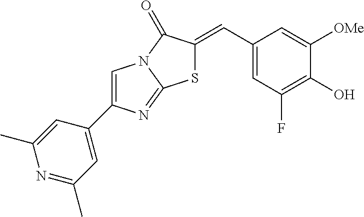 [SMILES: COc1cc(/C=c2\sc3nc(-c4cc(C)nc(C)c4)cn3c2=O)cc(F)c1O]

In analogy to the experimental procedure of example 9) 4-(2,6-dimethylpyridin-4-yl)-1H-imidazole-2(3H)-thione instead of 4-o-tolyl-1H-imidazole-2(3H)-thione was converted into the title compound (69 mg, 24%) which was obtained as a brown solid. MS m/e: 398.1 [M+H]⁺

### EXAMPLE 65

### 2-[1-(3-Bromo-4-hydroxy-5-methoxy-phenyl)-meth-(Z)-ylidene]-6-(2,6-dimethyl-pyridin-4-yl)-imidazo[2,1-b]thiazol-3-one

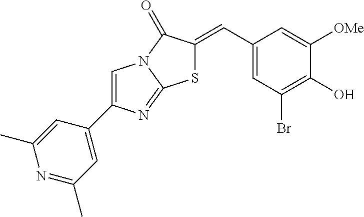 [SMILES: COc1cc(/C=c2\sc3nc(-c4cc(C)nc(C)c4)cn3c2=O)cc(Br)c1O]

In analogy to the experimental procedure of example 9) 4-(2,6-dimethylpyridin-4-yl)-1H-imidazole-2(3H)-thione instead of 4-o-tolyl-1H-imidazole-2(3H)-thione was converted using 3-bromo-4-hydroxy-5-methoxybenzaldehyde instead of 3-fluoro-4-hydroxy-5-methoxybenzaldehyde into the title compound (217 mg, 65%) which was obtained as a brown solid. MS m/e: 459.1 [M+H]⁺

### EXAMPLE 66

### (Z)-3-(2-(3-fluoro-4-hydroxy-5-methoxybenzylidene)-3-oxo-2,3-dihydroimidazo[2,1-b]thiazol-5-yl)benzonitrile

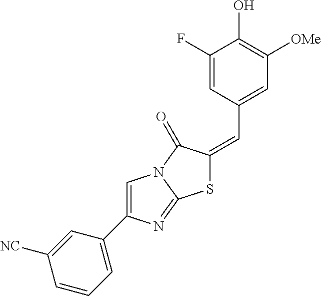 [SMILES: COc1cc(/C=c2/sc3nc(-c4cccc(C#N)c4)cn3c2=O)cc(F)c1O]

A suspension of (Z)-3-(2-(3-fluoro-4-hydroxy-5-methoxybenzylidene)-3-oxo-2,3-dihydroimidazo[2,1-b]thiazol-5-yl)benzonitrile (40 mg, 102 μmol) in methanol (30 mL) was stirred for 1 h at ambient temperature and then for 24 h at 50° C. The suspension was concentrated in vacuo and dried. The resulting red solid was suspended in acetonitrile (30 mL) at 50° C. After cooling to ambient temperature it was filtered over a satorius filter. Concentration in vacuo afforded the title compound (33 mg, 83%) as a red solid. MS m/e: 394.1 [M+H]⁺

### EXAMPLE 67

### 2-[1-(3-Chloro-4-hydroxy-5-methoxy-phenyl)-meth-(Z)-ylidene]-6-(3-(chloromethyl)phenyl)-imidazo[2,1-b]thiazol-3-one

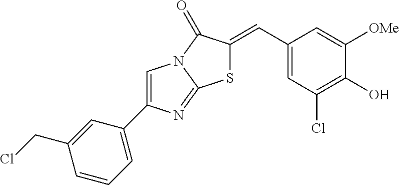 [SMILES: COc1cc(/C=c2\sc3nc(-c4cccc(CCl)c4)cn3c2=O)cc(Cl)c1O]

In analogy to the experimental procedure of example 43) 1-(3-(chloromethyl)phenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (26 mg, 17%) which was obtained as a yellow solid. MS m/e: 433.2 [M+H]⁺

### EXAMPLE 68

### 2-[1-(3-Chloro-4-hydroxy-5-methoxy-phenyl)-meth-(Z)-ylidene]-6-(pyridin-4-yl)-imidazo[2,1-b]thiazol-3-one

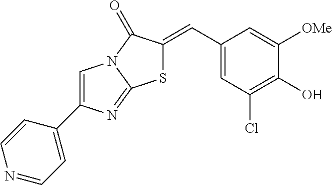 [SMILES: COc1cc(/C=c2\sc3nc(-c4ccncc4)cn3c2=O)cc(Cl)c1O]

In analogy to the experimental procedure of example 9) 4-(pyridin-4-yl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (74 mg, 23%) which was obtained as a brown solid. MS m/e: 486.1 [M+H]⁺

### EXAMPLE 69

### 2-[1-(3-Fluoro-4-hydroxy-5-methoxy-phenyl)-meth-(Z)-ylidene]-6-(pyridin-4-yl)-imidazo[2,1-b]thiazol-3-one

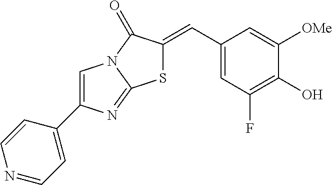 [SMILES: COc1cc(/C=c2\sc3nc(-c4ccncc4)cn3c2=O)cc(F)c1O]

In analogy to the experimental procedure of example 9) 4-(pyridin-4-yl)-1H-imidazole-2(3H)-thione instead of 4-o-tolyl-1H-imidazole-2(3H)-thione was converted into the title compound (74 mg, 24%) which was obtained as a brown solid. MS m/e: 370.1 [M+H]⁺

### EXAMPLE 70

### 2-[1-(3-Bromo-4-hydroxy-5-methoxy-phenyl)-meth-(Z)-ylidene]-6-(pyridin-4-yl)-imidazo[2,1-b]thiazol-3-one

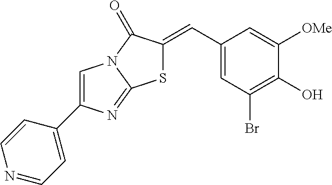 [SMILES: COc1cc(/C=c2\sc3nc(-c4ccncc4)cn3c2=O)cc(Br)c1O]

In analogy to the experimental procedure of example 9) 4-(pyridin-4-yl)-1H-imidazole-2(3H)-thione instead of 4-o-tolyl-1H-imidazole-2(3H)-thione was converted using 3-bromo-4-hydroxy-5-methoxybenzaldehyde instead of 3-fluoro-4-hydroxy-5-methoxybenzaldehyde into the title compound (95 mg, 26%) which was obtained as a brown solid. MS m/e: 431.1 [M+H]⁺

### EXAMPLE 71

### 2-[1-(3-Fluoro-4-hydroxy-5-methoxy-phenyl)-meth-(Z)-ylidene]-6-(pyridin-3-yl)-imidazo[2,1-b]thiazol-3-one

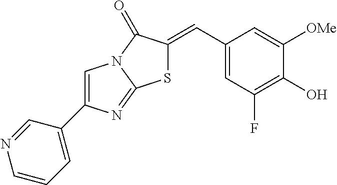 [SMILES: COc1cc(/C=c2\sc3nc(-c4cccnc4)cn3c2=O)cc(F)c1O]

In analogy to the experimental procedure of example 9) 4-(pyridin-3-yl)-1H-imidazole-2(3H)-thione instead of 4-o-tolyl-1H-imidazole-2(3H)-thione was converted into the title compound (101 mg, 32%) which was obtained as a brown solid. MS m/e: 370.1 [M+H]⁺

### EXAMPLE 72

### 2-[1-(3-Bromo-4-hydroxy-5-methoxy-phenyl)-meth-(Z)-ylidene]-6-(pyridin-3-yl)-imidazo[2,1-b]thiazol-3-one

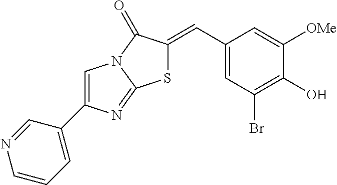 [SMILES: COc1cc(/C=c2\sc3nc(-c4cccnc4)cn3c2=O)cc(Br)c1O]

In analogy to the experimental procedure of example 9) 4-(pyridin-3-yl)-1H-imidazole-2(3H)-thione instead of 4-o-tolyl-1H-imidazole-2(3H)-thione was converted using 3-bromo-4-hydroxy-5-methoxybenz aldehyde instead of 3-fluoro-4-hydroxy-5-methoxybenzaldehyde into the title compound (149 mg, 41%) which was obtained as a brown solid. MS m/e: 431.1 [M+H]⁺

### EXAMPLE 73

### 3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzonitrile

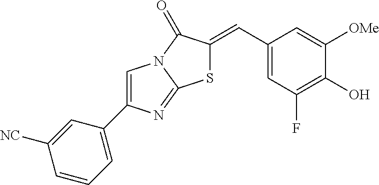 [SMILES: COc1cc(/C=c2\sc3nc(-c4cccc(C#N)c4)cn3c2=O)cc(F)c1O]

In analogy to the experimental procedure of example 1) 3-(2-thioxo-2,3-dihydro-1H-imidazol-4-yl)benzonitrile instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (126 mg, 17%) which was obtained as a brown solid. MS m/e: 378.9 [M+H]⁺

### EXAMPLE 74

### 2-[1-(3-Chloro-4-hydroxy-5-methoxy-phenyl)-meth-(Z)-ylidene]-6-(3-(2-fluoroethoxyl)phenyl)-imidazo[2,1-b]thiazol-3-one

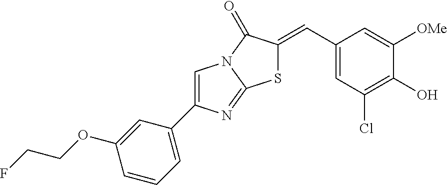 [SMILES: COc1cc(/C=c2\sc3nc(-c4cccc(OCCF)c4)cn3c2=O)cc(Cl)c1O]

In analogy to the experimental procedure of example 43) 1-(3-(2-fluoroethoxyl)phenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (4 mg, 3%) which was obtained as a yellow solid. MS m/e: 447.1 [M+H]⁺

### EXAMPLE 75

### 2-[1-(3-Chloro-4-hydroxy-5-methoxy-phenyl)-meth-(Z)-ylidene]-6-(2-methylpyridin-4-yl)-imidazo[2,1-b]thiazol-3-one

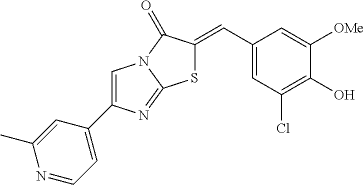 [SMILES: COc1cc(/C=c2\sc3nc(-c4ccnc(C)c4)cn3c2=O)cc(Cl)c1O]

In analogy to the experimental procedure of example 9) 4-(2-methylpyridin-4-yl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (38 mg, 12%) which was obtained as a brown solid. MS m/e: 400.1 [M+H]⁺

### EXAMPLE 76

### 2-[1-(3-Fluoro-4-hydroxy-5-methoxy-phenyl)-meth-(Z)-ylidene]-6-(2-methylpyridin-4-yl)-imidazo[2,1-b]thiazol-3-one

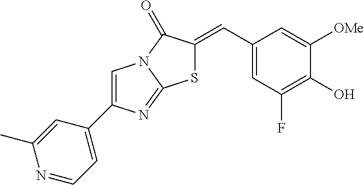 [SMILES: COc1cc(/C=c2\sc3nc(-c4ccnc(C)c4)cn3c2=O)cc(F)c1O]

In analogy to the experimental procedure of example 9) 4-(2-methylpyridin-4-yl)-1H-imidazole-2(3H)-thione instead of 4-o-tolyl-1H-imidazole-2(3H)-thione was converted into the title compound (60 mg, 20%) which was obtained as a brown solid. MS m/e: 384.1 [M+H]⁺

### EXAMPLE 77

### 2-[1-(3-Bromo-4-hydroxy-5-methoxy-phenyl)-meth-(Z)-ylidene]-6-(2-methylpyridin-4-yl)-imidazo[2,1-b]thiazol-3-one

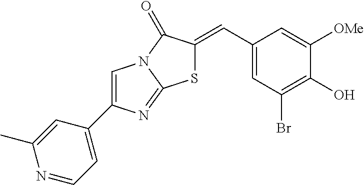 [SMILES: COc1cc(/C=c2\sc3nc(-c4ccnc(C)c4)cn3c2=O)cc(Br)c1O]

In analogy to the experimental procedure of example 9) 4-(2-methylpyridin-4-yl)-1H-imidazole-2(3H)-thione instead of 4-o-tolyl-1H-imidazole-2(3H)-thione was converted using 3-bromo-4-hydroxy-5-methoxybenzaldehyde instead of 3-fluoro-4-hydroxy-5-methoxybenzaldehyde into the title compound (111 mg, 32%) which was obtained as a brown solid. MS m/e: 445.1 [M+H]⁺

### EXAMPLE 78

### 2-[1-(3-Chloro-4-hydroxy-5-methoxy-phenyl)-meth-(Z)-ylidene]-6-(pyridin-3-yl)-imidazo[2,1-b]thiazol-3-one

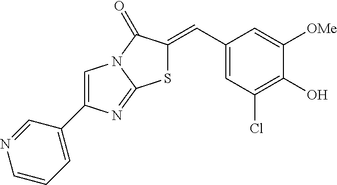 [SMILES: COc1cc(/C=c2\sc3nc(-c4cccnc4)cn3c2=O)cc(Cl)c1O]

In analogy to the experimental procedure of example 9) 4-(pyridin-3-yl)-1H-imidazole-2(3H)-thione instead of 4-o-tolyl-1H-imidazole-2(3H)-thione was converted using 3-chloro-4-hydroxy-5-methoxybenzaldehyde instead of 3-fluoro-4-hydroxy-5-methoxybenzaldehyde into the title compound (115 mg, 35%) which was obtained as a brown solid. MS m/e: 386.1 [M+H]⁺

### EXAMPLE 79

### 2-[1-(3-Chloro-4-hydroxy-5-methoxy-phenyl)-meth-(Z)-ylidene]-6-(4-(2-fluoroethoxyl)phenyl)-imidazo[2,1-b]thiazol-3-one

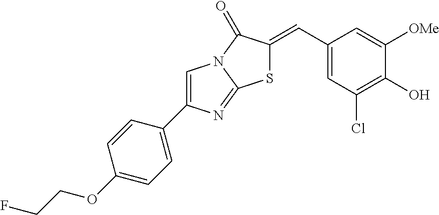 [SMILES: COc1cc(/C=c2\sc3nc(-c4ccc(OCCF)cc4)cn3c2=O)cc(Cl)c1O]

In analogy to the experimental procedure of example 43) 1-(4-(2-fluoroethoxyl)phenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (20 mg, 13%) which was obtained as a yellow solid. MS m/e: 447.1 [M+H]⁺

### EXAMPLE 80

### 2-[1-(3-Chloro-4-hydroxy-5-methoxy-phenyl)-meth-(Z)-ylidene]-6-(3-(allyloxy)phenyl)-imidazo[2,1-b]thiazol-3-one

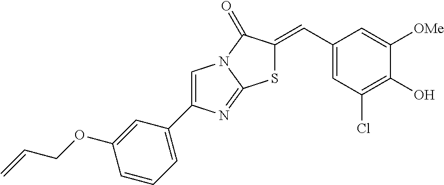 [SMILES: C=CCOc1cccc(-c2cn3c(=O)/c(=C/c4cc(Cl)c(O)c(OC)c4)sc3n2)c1]

In analogy to the experimental procedure of example 43) 1-(3-(allyloxy)phenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (42 mg, 16%) which was obtained as a yellow solid. MS m/e: 441.1 [M+H]⁺

### EXAMPLE 81

### 2-[1-(3-Chloro-4-hydroxy-5-methoxy-phenyl)-meth-(Z)-ylidene]-6-(3-propoxyphenyl)-imidazo[2,1-b]thiazol-3-one

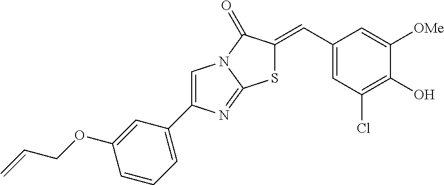 [SMILES: C=CCOc1cccc(-c2cn3c(=O)/c(=C/c4cc(Cl)c(O)c(OC)c4)sc3n2)c1]

In analogy to the experimental procedure of example 43) 1-(3-propoxyphenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (13 mg, 8%) which was obtained as a brown solid. MS m/e: 415.1 [M+H]⁺

### EXAMPLE 82

### 2-[1-(3-Chloro-4-hydroxy-5-methoxy-phenyl)-meth-(Z)-ylidene]-6-(2-(hydroxyethoxy)phenyl)-imidazo[2,1-b]thiazol-3-one

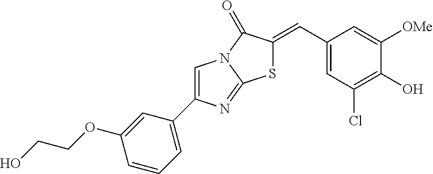 [SMILES: COc1cc(/C=c2\sc3nc(-c4cccc(OCCO)c4)cn3c2=O)cc(Cl)c1O]

In analogy to the experimental procedure of example 43) 1-(3-(2-hydroxyethoxy)phenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (23 mg, 9%) which was obtained as a yellow solid. MS m/e: 445.2 [M+H]⁺

### EXAMPLE 83

### 2-[1-(3-Chloro-4-hydroxy-5-methoxy-phenyl)-meth-(Z)-ylidene]-6-(2-(2-fluoroethoxyl)phenyl)-imidazo[2,1-b]thiazol-3-one

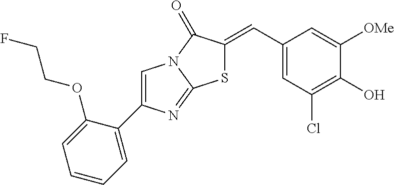 [SMILES: COc1cc(/C=c2\sc3nc(-c4ccccc4OCCF)cn3c2=O)cc(Cl)c1O]

In analogy to the experimental procedure of example 43) 1-(2-(2-fluoroethoxyl)phenyl)ethanone instead of acetophenone was converted using (Z)-2-amino-5-(3-chloro-4-hydroxy-5-methoxybenzylidene)thiazol-4(5H)-one instead of (Z)-2-amino-5-(3-fluoro-4-hydroxy-5-methoxybenzylidene)thiazol-4(5H)-one into the title compound (70 mg, 27%) which was obtained as a yellow solid. MS m/e: 445.1 [M+H]⁻

### EXAMPLE 84

### 2-[1-(3-Chloro-4-hydroxy-5-methoxy-phenyl)-meth-(Z)-ylidene]-6-(5-methyl-isoxazol-3-yl)-imidazo[2,1-b]thiazol-3-one

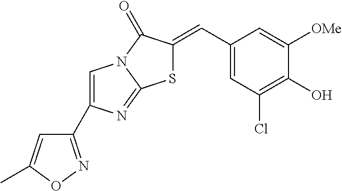 [SMILES: COc1cc(/C=c2\sc3nc(-c4cc(C)on4)cn3c2=O)cc(Cl)c1O]

In analogy to the experimental procedure of example 1) 4-(5-methylisoxazol-3-yl)-1H-imidazole-2(3H)-thione instead of 5-(pyridin-4-yl)-1H-imidazole-2-thiol was converted using 3-chloro-4-hydroxy-5-methoxybenzaldehyde instead of 4-hydroxy-3,5-dimethoxybenzaldehyde into the title compound (207 mg, 64%) which was obtained as a yellow solid. MS m/e: 390.1 [M+H]⁺

### EXAMPLE 85

### 3-Bromo-5-{2-[1-(3-chloro-4-hydroxy-5-methoxy-phenyl)-meth-(Z)-ylidene]-3-oxo-2,3-dihydro-imidazo[2,1-b]thiazol-6-yl}-benzonitrile

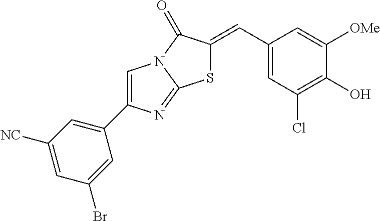 [SMILES: COc1cc(/C=c2\sc3nc(-c4cc(Br)cc(C#N)c4)cn3c2=O)cc(Cl)c1O]

To a mixture of (Z)-2-amino-5-(3-chloro-4-hydroxy-5-methoxybenzylidene)thiazol-4(5H)-one (200 mg, 702 μmol) and 3-bromo-5-(2-bromoacetyl)benzonitrile (213 mg, 702 μmol was added 2-propanol (4 mL). The reaction mixture was irradiated in the microwave at 160° C. for 30 min. The resulting yellow suspension was diluted with water (2 mL) and filtered off. The solid was washed twice with an aqueous solution of sodium hydrogen carbonate (1M, 2 mL), twice with an aqueous solution of citric acid (5%, 2 mL) and twice with water (3 mL) affording the title compound (218 mg, 64%) as a yellow solid. MS m/e: 490.0 [M+H]+

### EXAMPLE 86

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzamide

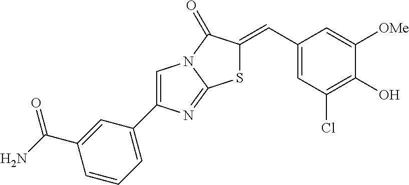 [SMILES: COc1cc(/C=c2\sc3nc(-c4cccc(C(N)=O)c4)cn3c2=O)cc(Cl)c1O]

In analogy to the experimental procedure of example 1) 3-(2-thioxo-2,3-dihydro-1H-imidazol-4-yl)benzamide instead of 5-(pyridin-4-yl)-1H-imidazole-2-thiol was converted using 3-chloro-4-hydroxy-5-methoxybenzaldehyde instead of 4-hydroxy-3,5-dimethoxybenzaldehyde into the title compound (102 mg, 29%) which was obtained as a yellow solid. MS m/e: 428.1 [M+H]⁺

### EXAMPLE 87

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzonitrile

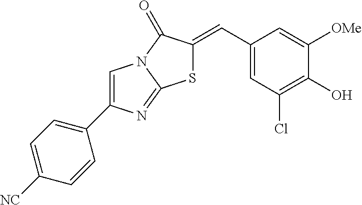 [SMILES: COc1cc(/C=c2\sc3nc(-c4ccc(C#N)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 4-(2-bromoacetyl)benzonitrile instead of 3-bromo-5-(2-bromoacetyl)benzonitrile into the title compound (81 mg, 56%) which was obtained as a yellow solid. MS m/e: 410.2 [M+H]⁺

### EXAMPLE 88

### 2-[1-(3-Chloro-4-hydroxy-5-methoxy-phenyl)-meth-(Z)-ylidene]-6-(4-(difluoromethoxy)phenyl)-imidazo[2,1-b]thiazol-3-one

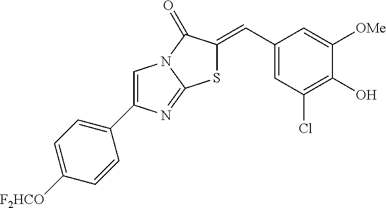 [SMILES: COc1cc(/C=c2\sc3nc(-c4ccc(OC(F)F)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(4-(difluoromethoxy)phenyl)ethanone instead of 3-bromo-5-(2-bromoacetyl)benzonitrile into the title compound (81 mg, 56%) which was obtained as a yellow solid. MS m/e: 410.2 [M+H]⁺

### EXAMPLE 89

### 2-[1-(3-Chloro-4-hydroxy-5-methoxy-phenyl)-meth-(Z)-ylidene]-6-(3-morpholinophenyl)-imidazo[2,1-b]thiazol-3-one

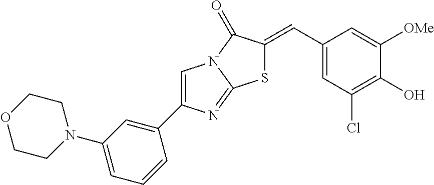 [SMILES: COc1cc(/C=c2\sc3nc(-c4cccc(N5CCOCC5)c4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(3-morpholinophenyl)ethanone instead of 3-bromo-5-(2-bromoacetyl)benzonitrile into the title compound (95 mg, 47%) which was obtained as a yellow solid. MS m/e: 470.1 [M+H]⁺

### EXAMPLE 90

### 2-[1-(3-Chloro-4-hydroxy-5-methoxy-phenyl)-meth-(Z)-ylidene]-6-(4-(pyrrolidin-1-yl)phenyl)-imidazo[2,1-b]thiazol-3-one

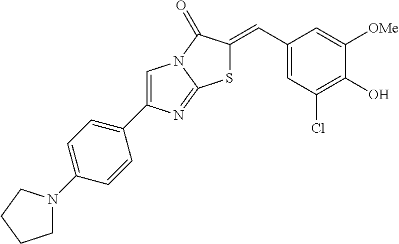 [SMILES: COc1cc(/C=c2\sc3nc(-c4ccc(N5CCCC5)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(4-(pyrrolidin-1-yl)phenyl)ethanone instead of 3-bromo-5-(2-bromoacetyl)benzonitrile into the title compound (56 mg, 29%) which was obtained as a brown solid. MS m/e: 454.2 [M+H]⁺

### EXAMPLE 91

### 2-[1-(3-Chloro-4-hydroxy-5-methoxy-phenyl)-meth-(Z)-ylidene]-6-(4-(diethylamino)phenyl)-imidazo[2,1-b]thiazol-3-one

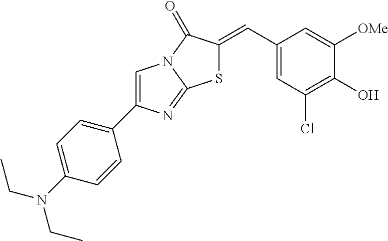 [SMILES: CCN(CC)c1ccc(-c2cn3c(=O)/c(=C/c4cc(Cl)c(O)c(OC)c4)sc3n2)cc1]

In analogy to the experimental procedure of example 85) (Z)-2-amino-5-(3-chloro-4-hydroxy-5-methoxybenzylidene)thiazol-4(5H)-one was converted using 2-bromo-1-(4-(diethylamino)phenyl)ethanone instead of 3-bromo-5-(2-bromoacetyl)benzonitrile into the title compound (89 mg, 54%) which was obtained as a brown solid. MS m/e: 456.2 [M+H]⁺

### EXAMPLE 92

### 2-[1-(3-Chloro-4-hydroxy-5-methoxy-phenyl)-meth-(Z)-ylidene]-6-(4-morpholinophenyl)-imidazo[2,1-b]thiazol-3-one

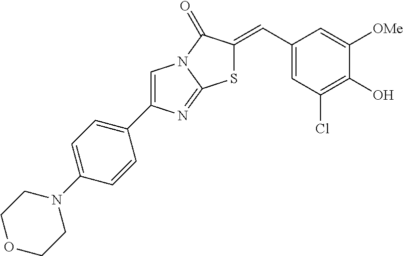 [SMILES: COc1cc(/C=c2\sc3nc(-c4ccc(N5CCOCC5)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(4-morpholinophenyl)ethanone instead of 3-bromo-5-(2-bromoacetyl)benzonitrile into the title compound (18 mg, 8%) which was obtained as a brown solid. MS m/e: 470.1 [M+H]⁺

### EXAMPLE 93

### 2-[1-(3-Chloro-4-hydroxy-5-methoxy-phenyl)-meth-(Z)-ylidene]-6-(4-(dimethylamino)phenyl)-imidazo[2,1-b]thiazol-3-one

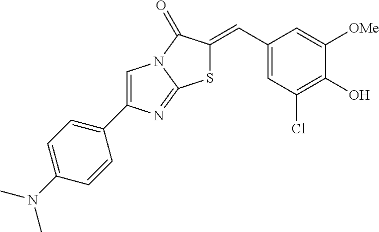 [SMILES: COc1cc(/C=c2\sc3nc(-c4ccc(N(C)C)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(4-(dimethylamino)phenyl)ethanone instead of 3-bromo-5-(2-bromoacetyl)benzonitrile into the title compound (37 mg, 17%) which was obtained as a brown solid. MS m/e: 428.2 [M+H]⁺

### EXAMPLE 94

### 2-[1-(3-Chloro-4-hydroxy-5-methoxy-phenyl)-meth-(Z)-ylidene]-6-[3-(2-oxo-pyrrolidin-1-yl)-phenyl]-imidazo[2,1-b]thiazol-3-one

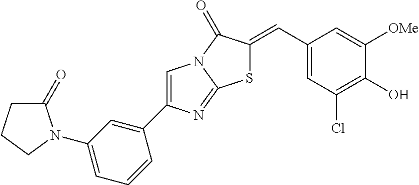 [SMILES: COc1cc(/C=c2\sc3nc(-c4cccc(N5CCCC5=O)c4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 1-(3-(2-bromoacetyl)phenyl)pyrrolidin-2-one instead of 3-bromo-5-(2-bromoacetyl)benzonitrile into the title compound (99 mg, 54%) which was obtained as an orange solid. MS m/e: 468.2 [M+H]⁺

### EXAMPLE 95

### 2-[1-(3-Chloro-4-hydroxy-5-methoxy-phenyl)-meth-(Z)-ylidene]-6-(3-{2-[2-(2-fluoro-ethoxy)-ethoxy]-ethoxy}-phenyl)-imidazo[2,1-b]thiazol-3-one

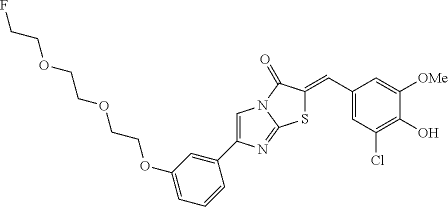 [SMILES: COc1cc(/C=c2\sc3nc(-c4cccc(OCCOCCOCCF)c4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(3-(2-(2-(2-fluoroethoxyl)ethoxy)ethoxy)phenyl)ethanone instead of 3-bromo-5-(2-bromoacetyl)benzonitrile into the title compound (24 mg, 31%) which was obtained as a yellow solid. MS m/e: 535.1 [M+H]⁺

### EXAMPLE 96

### 2-[1-(3-Chloro-4-hydroxy-5-methoxy-phenyl)-meth-(Z)-ylidene]-6-(2-{2-[2-(2-fluoro-ethoxy)-ethoxy]-ethoxy}-phenyl)-imidazo[2,1-b]thiazol-3-one

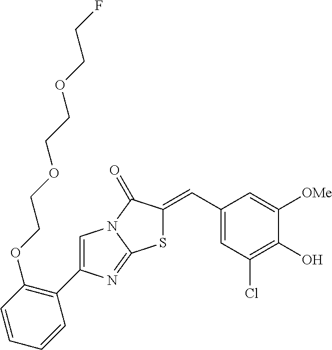 [SMILES: COc1cc(/C=c2\sc3nc(-c4ccccc4OCCOCCOCCF)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(2-(2-(2-(2-fluoroethoxyl)ethoxy)ethoxy)phenyl)ethanone instead of 3-bromo-5-(2-bromoacetyl)benzonitrile into the title compound (34 mg, 20%) which was obtained as a yellow solid. MS m/e: 470.1 [M+H]⁺

### EXAMPLE 97

### 2-[1-(3-Chloro-4-hydroxy-5-methoxy-phenyl)-meth-(Z)-ylidene]-6-[6-(4-methyl-3H-imidazol-1-yl)-pyridin-3-yl]-imidazo[2,1-b]thiazol-3-one

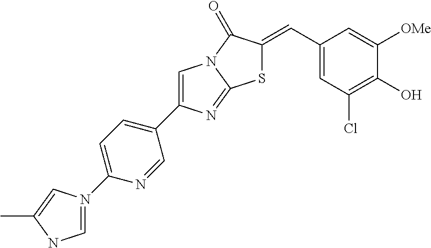

In analogy to the experimental procedure of example 85) (Z)-2-amino-5-(3-chloro-4-hydroxy-5-methoxybenzylidene)thiazol-4(5H)-one was converted using 2-bromo-1-(6-(4-methyl-1H-imidazol-1-yl)pyridin-3-yl)ethanone instead of 3-bromo-5-(2-bromoacetyl)benzonitrile into the title compound (94 mg, 69%) which was obtained as a yellow solid. MS m/e: 466.2 [M+H]⁺

### EXAMPLE 98

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid

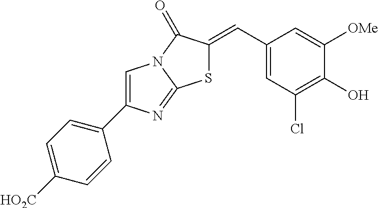 [SMILES: COc1cc(/C=c2\sc3nc(-c4ccc(C(=O)O)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 4-(2-bromoacetyl)benzoic acid instead of 3-bromo-5-(2-bromoacetyl)benzonitrile into the title compound (49 mg, 35%) which was obtained as a yellow solid. MS m/e: 427.1 [M+H]⁻

### EXAMPLE 99

### 2-[1-(3-Chloro-4-hydroxy-5-methoxy-phenyl)-meth-(Z)-ylidene]-6-(3,4-dimethoxy-phenyl)-imidazo[2,1-b]thiazol-3-one

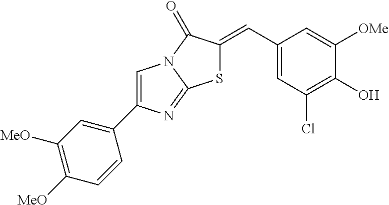 [SMILES: COc1ccc(-c2cn3c(=O)/c(=C/c4cc(Cl)c(O)c(OC)c4)sc3n2)cc1OC]

In analogy to the experimental procedure of example 85) (Z)-2-amino-5-(3-chloro-4-hydroxy-5-methoxybenzylidene)thiazol-4(5H)-one was converted using 2-bromo-1-(3,4-dimethoxyphenyl)ethanone instead of 3-bromo-5-(2-bromoacetyl)benzonitrile into the title compound (43 mg, 21%) which was obtained as a yellow solid. MS m/e: 443.2 [M+H]⁻

### EXAMPLE 100

### 2-[1-(3-Chloro-4-hydroxy-5-methoxy-phenyl)-meth-(Z)-ylidene]-6-(2,3-dihydro-benzo[1,4]dioxin-6-yl)-imidazo[2,1-b]thiazol-3-one

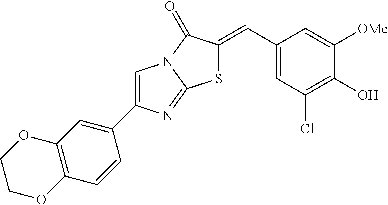 [SMILES: COc1cc(/C=c2\sc3nc(-c4ccc5c(c4)OCCO5)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(2,3-dihydrobenzo[b][1,4]dioxin-6-yl)ethanone instead of 3-bromo-5-(2-bromoacetyl)benzonitrile into the title compound (40 mg, 25%) which was obtained as a yellow solid. MS m/e: 443.2 [M+H]⁺

### EXAMPLE 101

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzamide

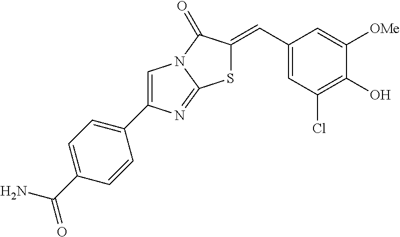 [SMILES: COc1cc(/C=c2\sc3nc(-c4ccc(C(N)=O)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 4-(2-bromoacetyl)benzamide instead of 3-bromo-5-(2-bromoacetyl)benzonitrile into the title compound (140 mg, 79%) which was obtained as a yellow solid. MS m/e: 426.1 [M+H]⁻

### EXAMPLE 102

### 5-{2-[1-(3-Chloro-4-hydroxy-5-methoxy-phenyl)-meth-(Z)-ylidene]-3-oxo-2,3-dihydro-imidazo[2,1-b]thiazol-6-yl}-1,3-dihydro-indol-2-one

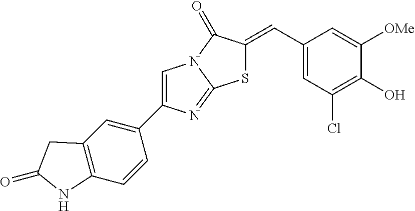 [SMILES: COc1cc(/C=c2\sc3nc(-c4ccc5c(c4)CC(=O)N5)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 5-(2-bromoacetyl)indolin-2-one instead of 3-bromo-5-(2-bromoacetyl)benzonitrile into the title compound (96 mg, 67%) which was obtained as an orange solid. MS m/e: 440.2 [M+H]⁺

### EXAMPLE 103

N-(4-{2-[1-(3-Chloro-4-hydroxy-5-methoxy-phenyl)-meth-(Z)-ylidene]-3-oxo-2,3-dihydro-imidazo[2,1-b]thiazol-6-yl}-phenyl)-acetamide

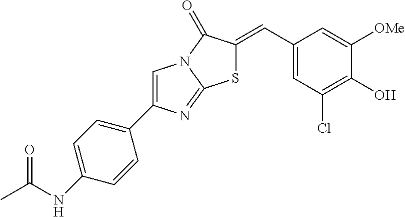 [SMILES: COc1cc(/C=c2\sc3nc(-c4ccc(NC(C)=O)cc4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using N-(4-(2-bromoacetyl)phenyl)acetamide instead of 3-bromo-5-(2-bromoacetyl)benzonitrile into the title compound (64 mg, 44%) which was obtained as an orange solid. MS m/e: 442.2 [M+H]⁺

### EXAMPLE 104

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid

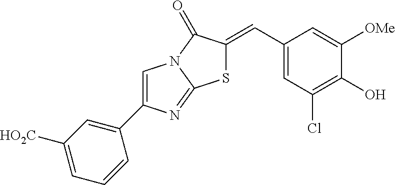 [SMILES: COc1cc(/C=c2\sc3nc(-c4cccc(C(=O)O)c4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 3-(2-bromoacetyl)benzoic acid instead of 3-bromo-5-(2-bromoacetyl)benzonitrile into the title compound (952 mg, 65%) which was obtained as a yellow solid. MS m/e: 429.1 [M+H]⁺

### EXAMPLE 105

### 2-[1-(3-Chloro-4-hydroxy-5-methoxy-phenyl)-meth-(Z)-ylidene]-6-(2,6-difluoro-phenyl)-imidazo[2,1-b]thiazol-3-one

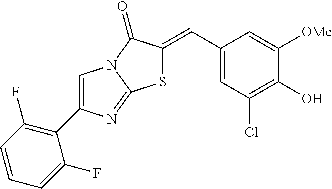 [SMILES: COc1cc(/C=c2\sc3nc(-c4c(F)cccc4F)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(2,6-difluorophenyl)ethanone instead of 3-bromo-5-(2-bromoacetyl)benzonitrile into the title compound (38 mg, 25%) which was obtained as a yellow solid. MS m/e: 421.0 [M+H]⁺

### EXAMPLE 106

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-benzamide

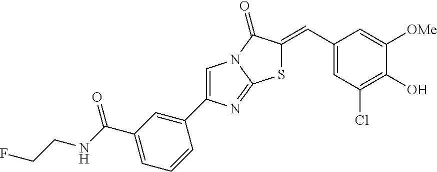 [SMILES: COc1cc(/C=c2\sc3nc(-c4cccc(C(=O)NCCF)c4)cn3c2=O)cc(Cl)c1O]

To a solution of (Z)-3-(2-(3-chloro-4-hydroxy-5-methoxybenzylidene)-3-oxo-2,3-dihydroimidazo[2,1-b]thiazol-5-yl)benzoic acid (96 mg, 224 μmol) in N-methyl-2-pyrrolidinone (1 mL) was added under a nitrogen atmosphere 2-(1H-benzo[d][1,2,3]triazol-1-yl)-1,1,3,3-tetramethylisouronium tetrafluoroborate (79.1 mg, 246 μmol) and 2-fluoroethylamine hydrochloride (22.3 mg, 224 μmol). After the addition of N,N-diisopropylethylamine (63.7 mg, 86.0 μL, 492 μmol) the color changed from yellow to red. The solution was stirred for 3 h at ambient temperature. The reaction mixture was diluted with dichloromethane (15 mL) and was washed with water (15 ml) and an aqueous solution of citric acid (5%, 15 mL). The aqueous layers were extracted three times with dichloromethane (15 mL). The combined organic layers were dried over sodium sulfate. Purification by chromatography (SiO₂, eluent:dichloromethane:ethyl acetate=100:0 to 70:30) afforded the title compound (28 mg, 26%) as a yellow solid. MS m/e: 474.1 [M+H]⁺

### EXAMPLE 107

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-propyl-benzamide

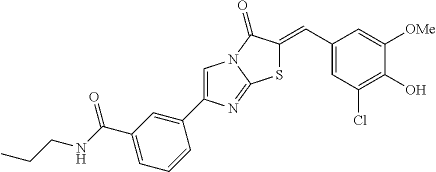 [SMILES: CCCNC(=O)c1cccc(-c2cn3c(=O)/c(=C/c4cc(Cl)c(O)c(OC)c4)sc3n2)c1]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using propan-1-amine instead of 2-fluoroethylamine hydrochloride into the title compound (35 mg, 27%) which was obtained as a yellow solid. MS m/e: 470.2 [M+H]⁺

### EXAMPLE 108

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-benzamide

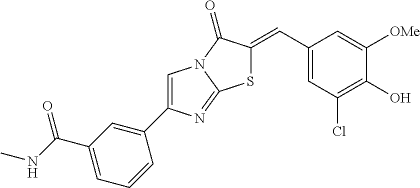 [SMILES: CNC(=O)c1cccc(-c2cn3c(=O)/c(=C/c4cc(Cl)c(O)c(OC)c4)sc3n2)c1]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using methylamine hydrochloride instead of 2-fluoroethylamine hydrochloride into the title compound (30 mg, 18%) which was obtained as a yellow solid. MS m/e: 442.1 [M+H]⁺

### EXAMPLE 109

### 2-[1-(3-Chloro-4-hydroxy-5-propoxy-phenyl)-meth-(Z)-ylidene]-6-(4-methyl-pyridin-3-yl)-imidazo[2,1-b]thiazol-3-one

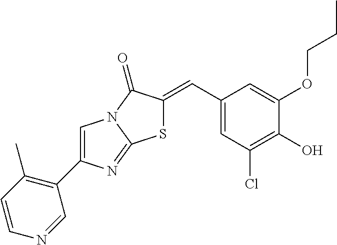 [SMILES: CCCOc1cc(/C=c2\sc3nc(-c4cnccc4C)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(4-methylpyridin-3-yl)ethanone hydrobromide instead of 3-bromo-5-(2-bromoacetyl)benzonitrile into the title compound (17 mg, 9%) which was obtained as a yellow solid. MS m/e: 428.1 [M+H]⁺

### EXAMPLE 110

### 2-[1-(3-Chloro-4-hydroxy-5-propoxy-phenyl)-meth-(Z)-ylidene]-6-pyridin-3-yl-imidazo[2,1-b]thiazol-3-one

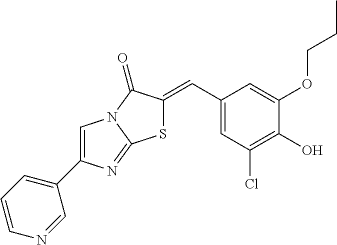 [SMILES: CCCOc1cc(/C=c2\sc3nc(-c4cccnc4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(pyridin-3-yl)ethanone hydrobromide instead of 3-bromo-5-(2-bromoacetyl)benzonitrile into the title compound (35 mg, 19%) which was obtained as a yellow solid. MS m/e: 414.2 [M+H]⁺

### EXAMPLE 111

### 2-[1-(3-Chloro-4-hydroxy-5-propoxy-phenyl)-meth-(Z)-ylidene]-6-pyridin-4-yl-imidazo[2,1-b]thiazol-3-one

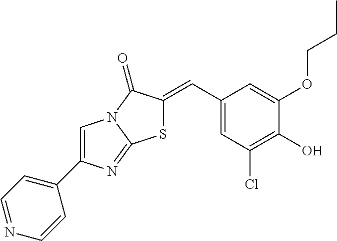 [SMILES: CCCOc1cc(/C=c2\sc3nc(-c4ccncc4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(pyridin-4-yl)ethanone hydrobromide instead of 3-bromo-5-(2-bromoacetyl)benzonitrile into the title compound (15 mg, 9%) which was obtained as a yellow solid. MS m/e: 414.1 [M+H]⁺

### EXAMPLE 112

### 2-[1-(3-Chloro-4-hydroxy-5-propoxy-phenyl)-meth-(Z)-ylidene]-6-[6-(4-methyl-imidazol-1-yl)-pyridin-3-yl]-imidazo[2,1-b]thiazol-3-one

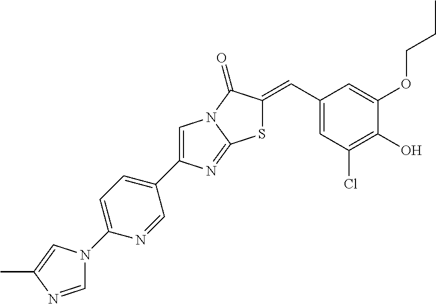 [SMILES: CCCOc1cc(/C=c2\sc3nc(-c4ccc(-n5cnc(C)c5)nc4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(6-(4-methyl-1H-imidazol-1-yl)pyridin-3-yl)ethanone acetate instead of 3-bromo-5-(2-bromoacetyl)benzonitrile into the title compound (39 mg, 23%) which was obtained as a yellow solid. MS m/e: 494.3 [M+H]⁺

### EXAMPLE 113

N-Allyl-3-{2-[1-(3-chloro-4-hydroxy-5-methoxy-phenyl)-meth-(Z)-ylidene]-3-oxo-2,3-dihydro-imidazo[2,1-b]thiazol-6-yl}-benzamide

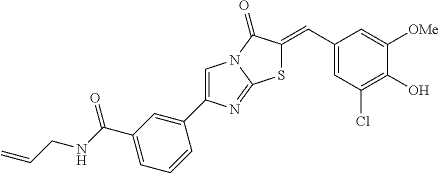 [SMILES: C=CCNC(=O)c1cccc(-c2cn3c(=O)/c(=C/c4cc(Cl)c(O)c(OC)c4)sc3n2)c1]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using prop-2-en-1-amine instead of 2-fluoroethylamine hydrochloride into the title compound (49 mg, 42%) which was obtained as a yellow solid. MS m/e: 468.2 [M+H]⁺

### EXAMPLE 114

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-benzamide

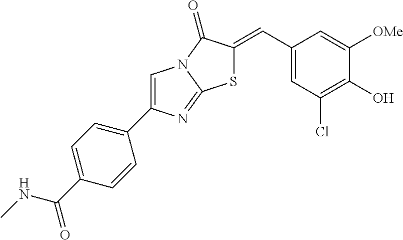 [SMILES: CNC(=O)c1ccc(-c2cn3c(=O)/c(=C/c4cc(Cl)c(O)c(OC)c4)sc3n2)cc1]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using methylamine hydrochloride instead of 2-fluoroethylamine hydrochloride into the title compound (27 mg, 18%) which was obtained as a yellow solid. MS m/e: 442.1 [M+H]⁺

### EXAMPLE 115

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-propyl-benzamide

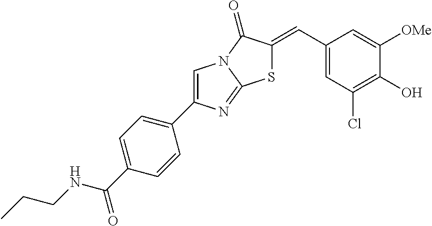 [SMILES: CCCNC(=O)c1ccc(-c2cn3c(=O)/c(=C/c4cc(Cl)c(O)c(OC)c4)sc3n2)cc1]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using propan-1-amine instead of 2-fluoroethylamine hydrochloride into the title compound (46 mg, 28%) which was obtained as a yellow solid. MS m/e: 470.2 [M+H]⁺

### EXAMPLE 116

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-benzamide

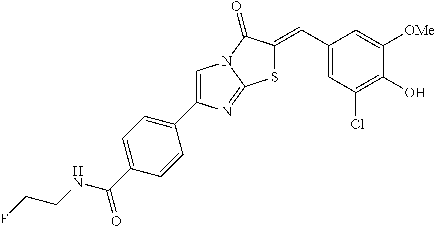 [SMILES: COc1cc(/C=c2\sc3nc(-c4ccc(C(=O)NCCF)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted into the title compound (80 mg, 48%) which was obtained as a yellow solid. MS m/e: 474.2 [M+H]⁺

### EXAMPLE 117

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoro-propyl)-benzamide

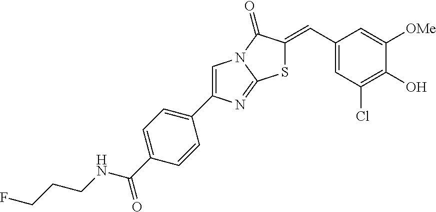 [SMILES: COc1cc(/C=c2\sc3nc(-c4ccc(C(=O)NCCCF)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoropropan-1-amine hydrochloride instead of 2-fluoroethylamine hydrochloride into the title compound (55 mg, 42%) which was obtained as a yellow solid. MS m/e: 488.2 [M+H]⁺

### EXAMPLE 118

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(3-fluoro-phenyl)-ethyl]-N-methyl-benzamide

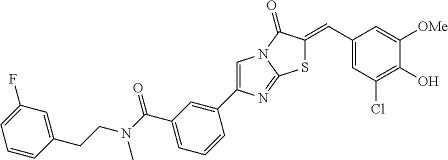 [SMILES: COc1cc(/C=c2\sc3nc(-c4cccc(C(=O)N(C)CCc5cccc(F)c5)c4)cn3c2=O)cc(Cl)c1O]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using [2-(3-fluoro-phenyl)-ethyl]-methyl-amine hydrochloride instead of 2-fluoroethylamine hydrochloride into the title compound (59 mg, 36%) which was obtained as a yellow solid. MS m/e: 564.3 [M+H]⁺

### EXAMPLE 119

### 2-[1-(3-Chloro-4-hydroxy-5-propoxy-phenyl)-meth-(Z)-ylidene]-6-(3H-imidazol-4-yl)-imidazo[2,1-b]thiazol-3-one

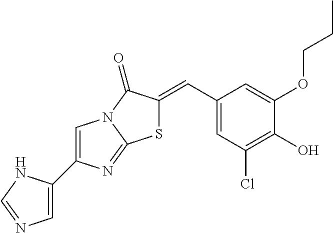 [SMILES: CCCOc1cc(/C=c2\sc3nc(-c4cnc[nH]4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(1H-imidazol-5-yl)ethanone hydrobromide instead of 3-bromo-5-(2-bromoacetyl)benzonitrile into the title compound (8 mg, 7%) which was obtained as a yellow solid. MS m/e: 403.3 [M+H]⁺

### EXAMPLE 120

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-N-methyl-benzamide

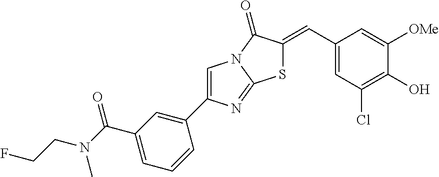 [SMILES: COc1cc(/C=c2\sc3nc(-c4cccc(C(=O)N(C)CCF)c4)cn3c2=O)cc(Cl)c1O]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using 2-fluoro-N-methylethanamine hydrochloride instead of 2-fluoroethylamine hydrochloride into the title compound (54 mg, 38%) which was obtained as a yellow solid. MS m/e: 488.1 [M+H]⁺

### EXAMPLE 121

### 3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzamide

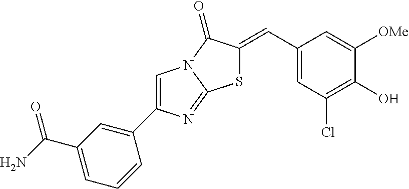 [SMILES: COc1cc(/C=c2\sc3nc(-c4cccc(C(N)=O)c4)cn3c2=O)cc(Cl)c1O]

In analogy to the experimental procedure of example 1) 3-(2-thioxo-2,3-dihydro-1H-imidazol-4-yl)benzamide instead of 5-(pyridin-4-yl)-1H-imidazole-2-thiol was converted using 3-fluoro-4-hydroxy-5-methoxybenzaldehyde instead of 4-hydroxy-3,5-dimethoxybenzaldehyde into the title compound (19 mg, 6%) which was obtained as a dark red solid. MS m/e: 412.2 [M+H]⁺

### EXAMPLE 122

### 2-[1-(3-Fluoro-4-hydroxy-5-propoxy-phenyl)-meth-(Z)-ylidene]-6-pyridin-4-yl-imidazo[2,1-b]thiazol-3-one

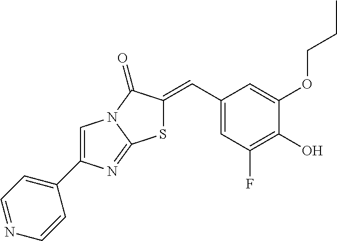 [SMILES: CCCOc1cc(/C=c2\sc3nc(-c4ccncc4)cn3c2=O)cc(F)c1O]

In analogy to the experimental procedure of example 9) 4-(pyridin-4-yl)-1H-imidazole-2(3H)-thione instead of 4-o-tolyl-1H-imidazole-2(3H)-thione was converted using 3-fluoro-4-hydroxy-5-methoxybenzaldehyde instead of 3-fluoro-4-hydroxy-5-methoxybenzaldehyde into the title compound (28 mg, 14%) which was obtained as a red solid. MS m/e: 398.2 [M+H]⁺

### EXAMPLE 123

### 2-[1-(3-Chloro-5-(3-fluoropropoxy)-4-hydroxy-phenyl)-meth-(Z)-ylidene]-6-pyridin-4-yl-imidazo[2,1-b]thiazol-3-one

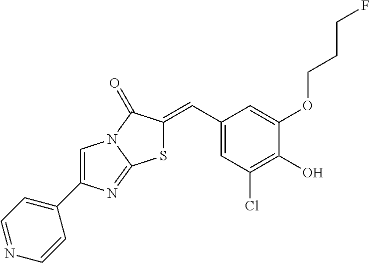 [SMILES: O=c1/c(=C/c2cc(Cl)c(O)c(OCCCF)c2)sc2nc(-c3ccncc3)cn12]

In analogy to the experimental procedure of example 9) 4-(pyridin-4-yl)-1H-imidazole-2(3H)-thione instead of 4-o-tolyl-1H-imidazole-2(3H)-thione was converted using 3-chloro-5-(3-fluoropropoxy)-4-hydroxybenzaldehyde instead of 3-fluoro-4-hydroxy-5-methoxybenzaldehyde into the title compound (32 mg, 13%) which was obtained as a red solid. MS m/e: 432.3 [M+H]⁺

### EXAMPLE 124

### 2-[1-(3-Fluoro-5-(3-fluoropropoxy)-4-hydroxy-phenyl)-meth-(Z)-ylidene]-6-pyridin-4-yl-imidazo[2,1-b]thiazol-3-one

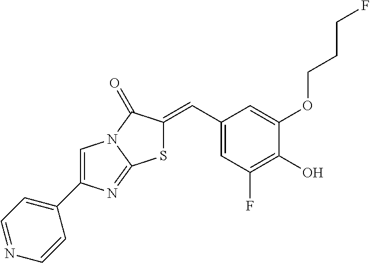 [SMILES: O=c1/c(=C/c2cc(F)c(O)c(OCCCF)c2)sc2nc(-c3ccncc3)cn12]

In analogy to the experimental procedure of example 9) 4-(pyridin-4-yl)-1H-imidazole-2(3H)-thione instead of 4-o-tolyl-1H-imidazole-2(3H)-thione was converted using 3-fluoro-5-(3-fluoropropoxy)-4-hydroxybenzaldehyde instead of 3-fluoro-4-hydroxy-5-methoxybenzaldehyde into the title compound (13 mg, 5%) which was obtained as a brown solid. MS m/e: 416.2 [M+H]⁺

### EXAMPLE 125

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-N-methyl-benzamide

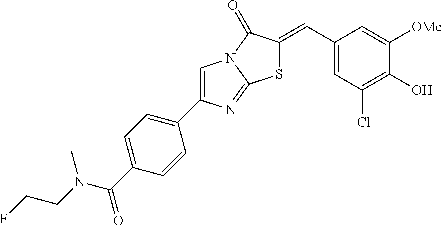 [SMILES: COc1cc(/C=c2\sc3nc(-c4ccc(C(=O)N(C)CCF)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 2-fluoro-N-methylethanamine hydrochloride instead of 2-fluoroethylamine hydrochloride into the title compound (45 mg, 36%) which was obtained as a red solid. MS m/e: 488.2 [M+H]⁺

### EXAMPLE 126

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N,N-dimethyl-benzamide

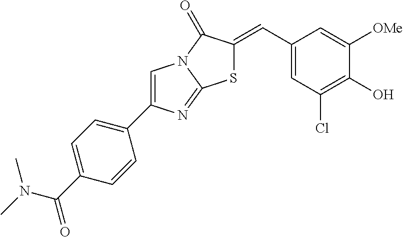 [SMILES: COc1cc(/C=c2\sc3nc(-c4ccc(C(=O)N(C)C)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using dimethylamine (2 M in tetrahydrofurane) instead of 2-fluoroethylamine hydrochloride into the title compound (43 mg, 38%) which was obtained as a yellow solid. MS m/e: 456.2 [M+H]⁺

### EXAMPLE 127

### 2-[1-(3-Chloro-4-hydroxy-5-methoxy-phenyl)-meth-(Z)-ylidene]-6-[4-(4-fluoro-piperidine-1-carbonyl)-phenyl]-imidazo[2,1-b]thiazol-3-one

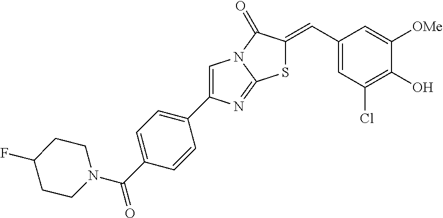 [SMILES: COc1cc(/C=c2\sc3nc(-c4ccc(C(=O)N5CCC(F)CC5)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 4-fluoropiperidine instead of 2-fluoroethylamine hydrochloride into the title compound (34 mg, 31%) which was obtained as a yellow solid. MS m/e: 514.3 [M+H]⁺

### EXAMPLE 128

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-N-propyl-benzamide

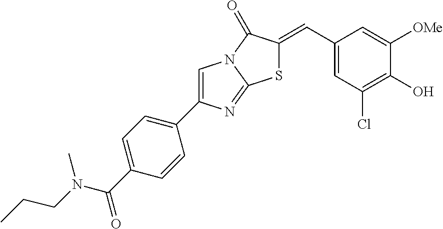 [SMILES: CCCN(C)C(=O)c1ccc(-c2cn3c(=O)/c(=C/c4cc(Cl)c(O)c(OC)c4)sc3n2)cc1]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using N-methylpropan-1-amine instead of 2-fluoroethylamine hydrochloride into the title compound (45 mg, 32%) which was obtained as a red solid. MS m/e: 484.3 [M+H]⁺

### EXAMPLE 129

### 2-[1-(3-Chloro-4-hydroxy-5-propoxy-phenyl)-meth-(Z)-ylidene]-6-pyrazin-2-yl-imidazo[2,1-b]thiazol-3-one

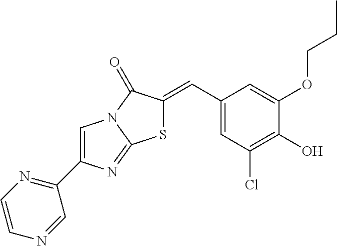 [SMILES: CCCOc1cc(/C=c2\sc3nc(-c4cnccn4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(pyrazin-2-yl)ethanone hydrobromide instead of 3-bromo-5-(2-bromoacetyl)benzonitrile into the title compound (8 mg, 6%) which was obtained as a yellow solid. MS m/e: 415.1 [M+H]⁺

### EXAMPLE 130

### 2-[1-(3-Chloro-4-hydroxy-5-propoxy-phenyl)-meth-(Z)-ylidene]-6-pyrimidin-5-yl-imidazo[2,1-b]thiazol-3-one

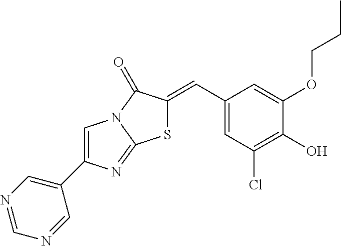 [SMILES: CCCOc1cc(/C=c2\sc3nc(-c4cncnc4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-chloro-1-(pyrimidin-5-yl)ethanone instead of 3-bromo-5-(2-bromoacetyl)benzonitrile into the title compound (1 mg, 1%) which was obtained as a yellow solid. MS m/e: 415.1 [M+H]⁺

### EXAMPLE 131

### 2-[1-(3-Chloro-4-hydroxy-5-propoxy-phenyl)-meth-(Z)-ylidene]-6-thiophen-3-yl-imidazo[2,1-b]thiazol-3-one

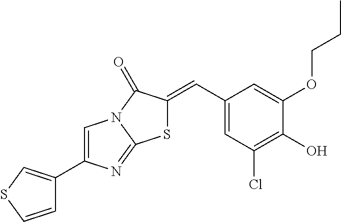 [SMILES: CCCOc1cc(/C=c2\sc3nc(-c4ccsc4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(thiophen-3-yl)ethanone instead of 3-bromo-5-(2-bromoacetyl)benzonitrile into the title compound (51 mg, 32%) which was obtained as a yellow solid. MS m/e: 419.1 [M+H]⁺

### EXAMPLE 132

### 2-[1-(3-Chloro-4-hydroxy-5-propoxy-phenyl)-meth-(Z)-ylidene]-6-thiophen-2-yl-imidazo[2,1-b]thiazol-3-one

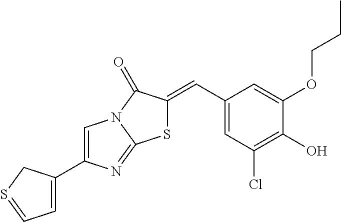

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(thiophen-2-yl)ethanone instead of 3-bromo-5-(2-bromoacetyl)benzonitrile into the title compound (58 mg, 37%) which was obtained as a yellow solid. MS m/e: 419.1 [M+H]⁺

### EXAMPLE 133

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoropropoxy)-benzamide

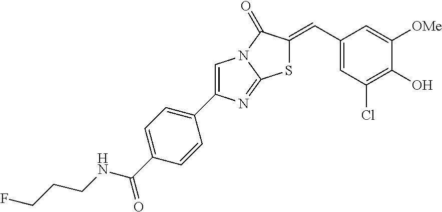 [SMILES: COc1cc(/C=c2\sc3nc(-c4ccc(C(=O)NCCCF)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoropropan-1-amine hydrochloride instead of 2-fluoroethylamine hydrochloride into the title compound (39 mg, 31%) which was obtained as a red solid. MS m/e: 488.2 [M+H]⁺

### EXAMPLE 134

### 2-[1-(3-Chloro-4-hydroxy-5-methoxy-phenyl)-meth-(Z)-ylidene]-6-[4-(3-fluoro-azetidine-1-carbonyl)-phenyl]-imidazo[2,1-b]thiazol-3-one

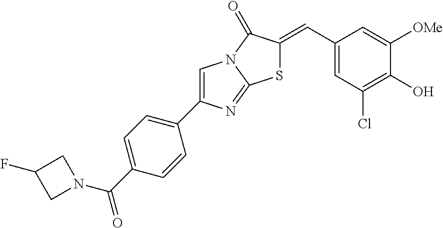 [SMILES: COc1cc(/C=c2\sc3nc(-c4ccc(C(=O)N5CC(F)C5)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoroazetidine hydrochloride instead of 2-fluoroethylamine hydrochloride into the title compound (49 mg, 33%) which was obtained as a red solid. MS m/e: 486.3 [M+H]⁺

### EXAMPLE 135

### 2-[1-(3-Chloro-4-hydroxy-5-propoxy-phenyl)-meth-(Z)-ylidene]-6-(2,4-dimethylthiophen-3-yl)-imidazo[2,1-b]thiazol-3-one

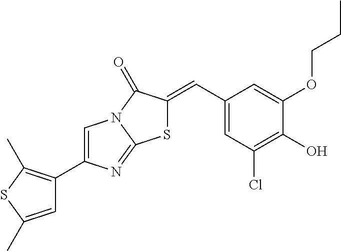 [SMILES: CCCOc1cc(/C=c2\sc3nc(-c4cc(C)sc4C)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(2,4-dimethylthiophen-3-yl)ethanone instead of 3-bromo-5-(2-bromoacetyl)benzonitrile into the title compound (34 mg, 23%) which was obtained as a yellow solid. MS m/e: 447.1 [M+H]⁺

### EXAMPLE 136

### 2-[1-(3-Chloro-4-hydroxy-5-propoxy-phenyl)-meth-(Z)-ylidene]-6-pyridin-2-yl-imidazo[2,1-b]thiazol-3-one

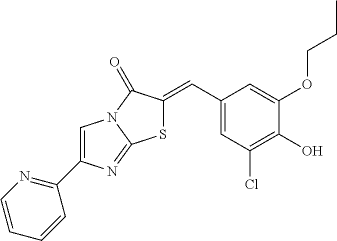 [SMILES: CCCOc1cc(/C=c2\sc3nc(-c4ccccn4)cn3c2=O)cc(Cl)c1O]

In analogy to the experimental procedure of example 85) (Z)-2-amino-5-(3-chloro-4-hydroxy-5-propoxybenzylidene)thiazol-4(5H)-one instead of (Z)-2-amino-5-(3-chloro-4-hydroxy-5-methoxybenzylidene)thiazol-4(5H)-one was converted using 2-bromo-1-(pyridin-2-yl)ethanone hydrobromide instead of 3-bromo-5-(2-bromoacetyl)benzonitrile into the title compound (19 mg, 13%) which was obtained as a yellow solid. MS m/e: 414.2 [M+H]⁺

### EXAMPLE 137

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoropropoxy)-N-methyl-benzamide

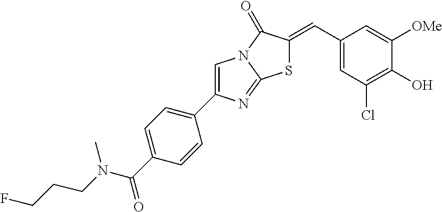 [SMILES: COc1cc(/C=c2\sc3nc(-c4ccc(C(=O)N(C)CCCF)cc4)cn3c2=O)cc(Cl)c1O]

In analogy to the experimental procedure of example 105) (Z)-4-(2-(3-chloro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoro-N-methylpropan-1-amine hydrochloride instead of 2-fluoroethylamine hydrochloride into the title compound (49 mg, 38%) which was obtained as a yellow solid. MS m/e: 502.2 [M+H]⁺

### EXAMPLE 138

### 4-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzoic acid

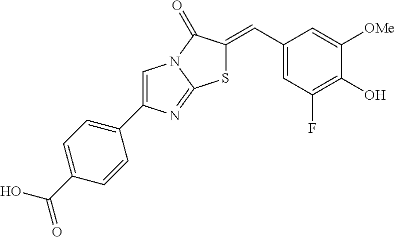 [SMILES: COc1cc(/C=c2\sc3nc(-c4ccc(C(=O)O)cc4)cn3c2=O)cc(F)c1O]

In analogy to the experimental procedure of example 85) (Z)-2-amino-5-(3-fluoro-4-hydroxy-5-methoxybenzylidene)thiazol-4(5H)-one instead of (Z)-2-amino-5-(3-chloro-4-hydroxy-5-methoxybenzylidene)thiazol-4(5H)-one was converted using 4-(2-bromoacetyl)benzoic acid instead of 3-bromo-5-(2-bromoacetyl)benzonitrile into the title compound (497 mg, 53%) which was obtained as a yellow solid. MS m/e: 413.2 [M+H]⁺

### EXAMPLE 139

### 6-[4-(3-Fluoro-azetidine-1-carbonyl)-phenyl]-2-[1-(3-fluoro-4-hydroxy-5-methoxy-phenyl)-meth-(Z)-ylidene]-imidazo[2,1-b]thiazol-3-one

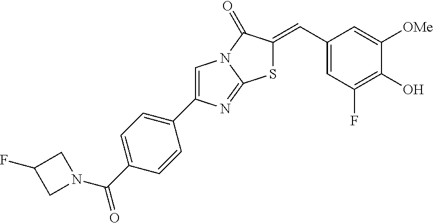 [SMILES: COc1cc(/C=c2\sc3nc(-c4ccc(C(=O)N5CC(F)C5)cc4)cn3c2=O)cc(F)c1O]

In analogy to the experimental procedure of example 105) (Z)-4-(2-(3-fluoro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoroazetidine hydrochloride instead of 2-fluoroethylamine hydrochloride into the title compound (72 mg, 59%) which was obtained as a yellow solid. MS m/e: 470.2 [M+H]⁺

### EXAMPLE 140

### 4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoroethoxy)-benzamide

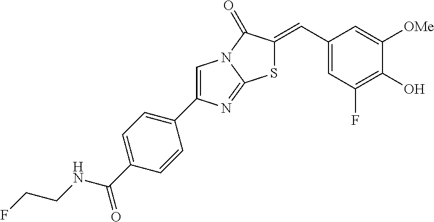 [SMILES: COc1cc(/C=c2\sc3nc(-c4ccc(C(=O)NCCF)cc4)cn3c2=O)cc(F)c1O]

In analogy to the experimental procedure of example 105) (Z)-4-(2-(3-fluoro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted into the title compound (40 mg, 33%) which was obtained as a yellow solid. MS m/e: 458.3 [M+H]⁺

### EXAMPLE 141

### 3-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(3-fluoropropoxy)-N-methyl-benzamide

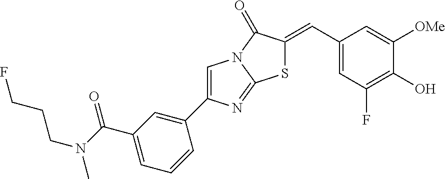 [SMILES: COc1cc(/C=c2\sc3nc(-c4cccc(C(=O)N(C)CCCF)c4)cn3c2=O)cc(F)c1O]

In analogy to the experimental procedure of example 105) (Z)-4-(2-(3-fluoro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoro-N-methylpropan-1-amine hydrochloride instead of 2-fluoroethanamine hydrochloride into the title compound (53 mg, 37%) which was obtained as a yellow solid. MS m/e: 502.2 [M+H]⁺

### EXAMPLE 142

### 2-[1-(3-Chloro-4-hydroxy-5-methoxy-phenyl)-meth-(Z)-ylidene]-6-[3-(3-fluoro-azetidine-1-carbonyl)-phenyl]imidazo[2,1-b]thiazol-3-one

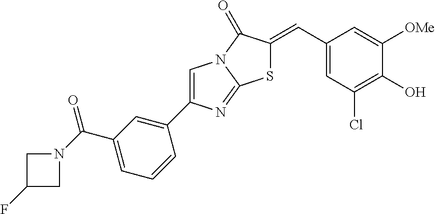 [SMILES: COc1cc(/C=c2\sc3nc(-c4cccc(C(=O)N5CC(F)C5)c4)cn3c2=O)cc(Cl)c1O]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using 3-fluoroazetidine hydrochloride instead of 2-fluoroethylamine hydrochloride into the title compound (73 mg, 52%) which was obtained as a yellow solid. MS m/e: 486.2 [M+H]⁺

### EXAMPLE 143

### 2-[1-(3-Chloro-4-hydroxy-5-methoxy-phenyl)-meth-(Z)-ylidene]-6-[3-(4-fluoro-piperidine-1-carbon-phenyl]imidazo[2,1-b]thiazol-3-one

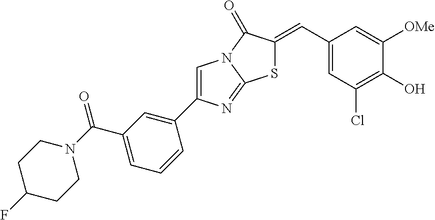 [SMILES: COc1cc(/C=c2\sc3nc(-c4cccc(C(=O)N5CCC(F)CC5)c4)cn3c2=O)cc(Cl)c1O]

In analogy to the experimental procedure of example 105) of (Z)-3-(2-(3-chloro-4-hydroxy-5-methoxybenzylidene)-3-oxo-2,3-dihydroimidazo[2,1-b]thiazol-5-yl)benzoic acid was converted using 4-fluoropiperidine hydrochloride instead of 2-fluoroethylamine hydrochloride into the title compound (52 mg, 38%) which was obtained as a yellow solid. MS m/e: 514.3 [M+H]⁺

### EXAMPLE 144

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(2-fluoro-ethoxy)-ethyl]-N-methyl-benzamide

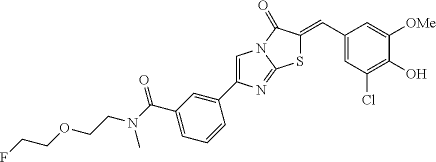 [SMILES: COc1cc(/C=c2\sc3nc(-c4cccc(C(=O)N(C)CCOCCF)c4)cn3c2=O)cc(Cl)c1O]

In analogy to the experimental procedure of example 105) of (Z)-3-(2-(3-chloro-4-hydroxy-5-methoxybenzylidene)-3-oxo-2,3-dihydroimidazo[2,1-b]thiazol-5-yl)benzoic acid was converted using 2-(2-fluoroethoxy)-N-methylethanamine hydrochloride instead of 2-fluoroethylamine hydrochloride into the title compound (38 mg, 26%) which was obtained as a yellow solid. MS m/e: 532.1 [M+H]⁺

### EXAMPLE 145

### 2-[1-(3-Chloro-4-hydroxy-5-methoxy-phenyl)-meth-(Z)-ylidene]-6-(2-chloro-pyridin-4-yl)-imidazo[2,1-b]thiazol-3-one

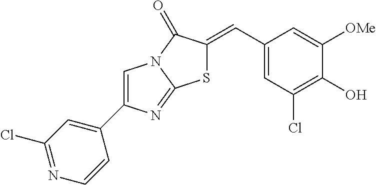 [SMILES: COc1cc(/C=c2\sc3nc(-c4ccnc(Cl)c4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 2-bromo-1-(2-chloropyridin-4-yl)ethanone instead of 3-bromo-5-(2-bromoacetyl)benzonitrile into the title compound (214 mg, 73%) which was obtained as a yellow solid. MS m/e: 420.0 [M+H]⁺

### EXAMPLE 146

### 4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoroethoxy)-N-methyl-benzamide

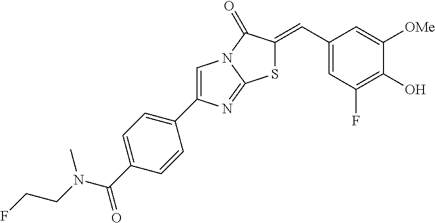 [SMILES: COc1cc(/C=c2\sc3nc(-c4ccc(C(=O)N(C)CCF)cc4)cn3c2=O)cc(F)c1O]

In analogy to the experimental procedure of example 105) (Z)-4-(2-(3-fluoro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 2-fluoro-N-methylethanamine hydrochloride instead of 2-fluoroethanamine hydrochloride into the title compound (69 mg, 66%) which was obtained as a yellow solid. MS m/e: 472.3 [M+H]⁺

### EXAMPLE 147

### 4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoropropoxy)-N-methyl-benzamide

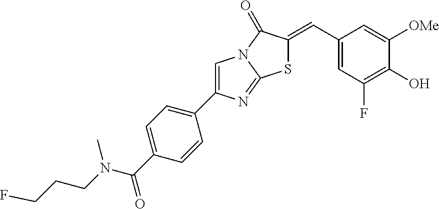 [SMILES: COc1cc(/C=c2\sc3nc(-c4ccc(C(=O)N(C)CCCF)cc4)cn3c2=O)cc(F)c1O]

In analogy to the experimental procedure of example 105) (Z)-4-(2-(3-fluoro-4-hydroxy-5-methoxybenzylidene)-3-oxo-2,3-dihydroimidazo[2,1-b]thiazol-5-yl)benzoic acid instead of (Z)-3-(2-(3-chloro-4-hydroxy-5-methoxybenzylidene)-3-oxo-2,3-dihydroimidazo[2,1-b]thiazol-5-yl)benzoic acid was converted using 3-fluoro-N-methylpropan-1-amine hydrochloride instead of 2-fluoroethanamine hydrochloride into the title compound (49 mg, 46%) which was obtained as a yellow solid. MS m/e: 486.4 [M+H]⁺

### EXAMPLE 148

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid 2-fluoro-ethyl ester

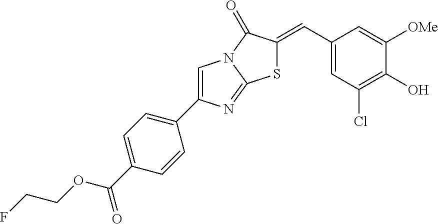 [SMILES: COc1cc(/C=c2\sc3nc(-c4ccc(C(=O)OCCF)cc4)cn3c2=O)cc(Cl)c1O]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using 2-fluoroethanol instead of 2-fluoroethanamine hydrochloride into the title compound (26 mg, 19%) which was obtained as an orange solid. MS m/e: 475.1 [M+H]⁺

### EXAMPLE 149

### 4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid 3-fluoro-propyl ester

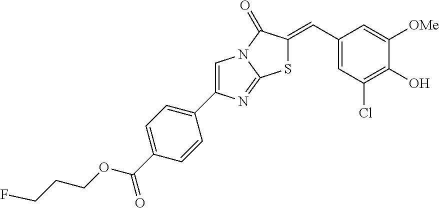 [SMILES: COc1cc(/C=c2\sc3nc(-c4ccc(C(=O)OCCCF)cc4)cn3c2=O)cc(Cl)c1O]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using 3-fluoropropan-1-ol instead of 2-fluoroethanamine hydrochloride into the title compound (15 mg, 12%) which was obtained as an orange solid. MS m/e: 489.3 [M+H]⁺

### EXAMPLE 150

[2-(4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoylamino)-ethyl]-carbamic acid tert-butyl ester

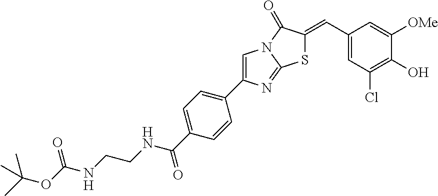 [SMILES: COc1cc(/C=c2\sc3nc(-c4ccc(C(=O)NCCNC(=O)OC(C)(C)C)cc4)cn3c2=O)cc(Cl)c1O]

In analogy to the experimental procedure of example 105) (Z)-3-(2-(3-chloro-4-hydroxy-5-methoxybenzylidene)-3-oxo-2,3-dihydroimidazo[2,1-b]thiazol-5-yl)benzoic acid was converted using tert-butyl 2-aminoethylcarbamate instead of 2-fluoroethanamine hydrochloride into the title compound (101 mg, 38%) which was obtained as a yellow solid. MS m/e: 571.2 [M+H]⁺

### EXAMPLE 151

### N-(2-Amino-ethyl)-4-{2-[1-(3-chloro-4-hydroxy-5-methoxy-phenyl)-meth-(Z)-ylidene]-3-oxo-2,3-dihydro-imidazo[2,1-b]thiazol-6-yl}-benzamide

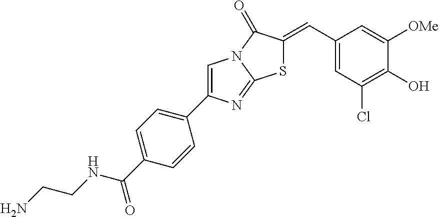 [SMILES: COc1cc(/C=c2\sc3nc(-c4ccc(C(=O)NCCN)cc4)cn3c2=O)cc(Cl)c1O]

To a suspension of (Z)-tert-butyl 2-(4-(2-(3-chloro-4-hydroxy-5-methoxybenzylidene)-3-oxo-2,3-dihydroimidazo[2,1-b]thiazol-5-yl)benzamido)ethylcarbamate (95 mg, 166 μmol) in tetrahydrofurane (2 mL) was added under an atmosphere of nitrogen aqueous hydrochloric acid (4 M in dioxane, 416 μl, 1.66 mmol). The reaction mixture was stirred at ambient temperature for 8 h. The suspension was centrifuged and the upper layer was pipetted off. Drying of the residue in vacuo afforded the title compound (76 mg, 90%) as a yellow solid. MS m/e: 469.07 [M−H]⁻

### EXAMPLE 152

### 3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzenesulfonyl fluoride

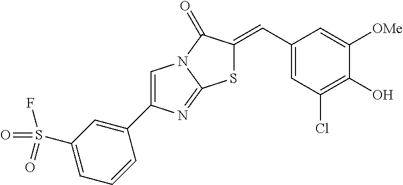 [SMILES: COc1cc(/C=c2\sc3nc(-c4cccc(S(=O)(=O)F)c4)cn3c2=O)cc(Cl)c1O]

In analogy to the experimental procedure of example 85) (Z)-2-amino-5-(3-chloro-4-hydroxy-5-methoxybenzylidene)thiazol-4(5H)-one was converted using 3-(2-bromoacetyl)benzene-1-sulfonyl fluoride instead of 3-bromo-5-(2-bromoacetyl)benzonitrile into the title compound (82 mg, 28%) which was obtained as a yellow solid. MS m/e: 467.2 [M+H]⁺

### EXAMPLE 153

### 2-[1-[3-Chloro-5-(3-fluoro-propoxy)-4-hydroxy-phenyl]-meth-(Z)-ylidene]-6-(2,6-dimethyl-pyridin-4-yl)-imidazo[2,1-b]thiazol-3-one

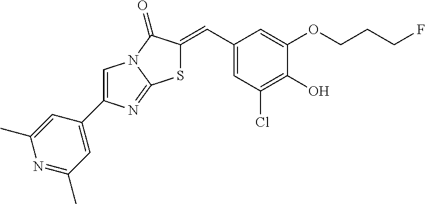 [SMILES: Cc1cc(-c2cn3c(=O)/c(=C/c4cc(Cl)c(O)c(OCCCF)c4)sc3n2)cc(C)n1]

In analogy to the experimental procedure of example 9) 4-(2,6-dimethylpyridin-4-yl)-1H-imidazole-2(3H)-thione instead of 4-o-tolyl-1H-imidazole-2(3H)-thione was converted using 3-chloro-5-(3-fluoropropoxy)-4-hydroxybenzaldehyde instead of 3-fluoro-4-hydroxy-5-methoxybenzaldehyde into the title compound (83 mg, 22%) which was obtained as a red solid. MS m/e: 460.5 [M+H]⁺

### EXAMPLE 154

### 3-{2-[1-[3-Chloro-5-(3-fluoro-propoxy)-4-hydroxy-phenyl]-meth-(Z)-ylidene]-3-oxo-2,3-dihydro-imidazo[2,1-b]thiazol-6-yl}-benzonitrile

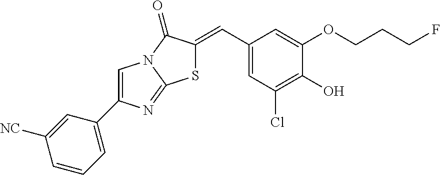 [SMILES: N#Cc1cccc(-c2cn3c(=O)/c(=C/c4cc(Cl)c(O)c(OCCCF)c4)sc3n2)c1]

In analogy to the experimental procedure of example 9) 3-(2-thioxo-2,3-dihydro-1H-imidazol-4-yl)benzonitrile instead of 4-o-tolyl-1H-imidazole-2(3H)-thione was converted using 3-chloro-5-(3-fluoropropoxy)-4-hydroxybenzaldehyde instead of 3-fluoro-4-hydroxy-5-methoxybenzaldehyde into the title compound (35 mg, 8%) which was obtained as a yellow solid. MS m/e: 456.5 [M+H]⁺

1. A compound of formula 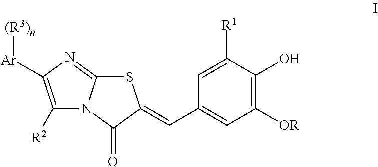
wherein
Ar is phenyl, pyridinyl, 2,3-dihydro-benzo[1,4]dioxinyl, 1,3-dihydro-indol-2-one, pyrazinyl, isoxazol-3-yl, imidazolyl, thiophenyl or pyrimidinyl;
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
R³ is hydrogen, halogen, lower alkyl, lower alkyl substituted by halogen, lower alkoxy, lower alkoxy substituted by halogen, O(CH₂)ₘO(CH₂)ₘO-lower alkyl substituted by halogen, cyano, lower alkoxy substituted by hydroxy, lower alkenyloxy, C(O)OH, heterocycloalkyl selected from morpholinyl, pyrrolidinyl or pyrrolidin-2-one, or is heteroaryl selected from imidazolyl substituted by lower alkyl, or is
NR′R″ and R′/R″ are independently from each other hydrogen or lower alkyl or —C(O)-lower alkyl; or is
—C(O)NR⁴R⁵ and
R⁴ is hydrogen or lower alkyl and
R⁵ is hydrogen, lower alkyl, lower alkenyl, —(CH₂)ₘO-lower alkyl substituted by halogen, lower alkyl substituted by halogen, —(CH₂)ₙ-phenyl optionally substituted by halogen, —CH₂)ₘNHC(O)-lower alkyl, or —(CH₂)ₘNH₂, or
R⁴ and R⁵ may form together with the N-atom to which they are attached a piperidine or azetidine ring, which may be substituted by halogen; or is
—C(O)O-lower alkyl substituted by halogen;
n is 1 or 2;
m is 1, 2 or 3;
or a pharmaceutically acceptable acid addition salt, a racemic mixture or its corresponding enantiomer and/or optical isomers thereof.

2. A compound according to claim 1, further defined as a compound of formula IA 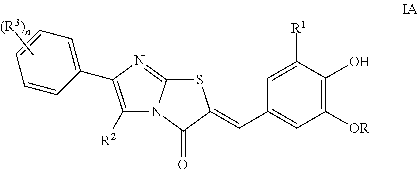
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
R³ is hydrogen, halogen, lower alkyl, lower alkyl substituted by halogen, lower alkoxy, lower alkoxy substituted by halogen, O(CH₂)ₘO(CH₂)ₘO-lower alkyl substituted by halogen, cyano, lower alkoxy substituted by hydroxy, lower alkenyloxy, C(O)OH, heterocycloalkyl selected from morpholinyl, pyrrolidinyl or pyrrolidin-2-one, or is heteroaryl selected from imidazolyl substituted by lower alkyl, or is
NR′R″ and R′/R″ are independently from each other hydrogen or lower alkyl or —C(O)-lower alkyl; or is
—C(O)NR⁴R⁵ and
R⁴ is hydrogen or lower alkyl and
R⁵ is hydrogen, lower alkyl, lower alkenyl, —(CH₂)ₘO-lower alkyl substituted by halogen, lower alkyl substituted by halogen, —(CH₂)ₙ-phenyl optionally substituted by halogen, —CH₂)ₘNHC(O)-lower alkyl, or —(CH₂)ₘNH₂, or
R⁴ and R⁵ may form together with the N-atom to which they are attached a piperidine or azetidine ring, which may be substituted by halogen; or is
—C(O)O-lower alkyl substituted by halogen;
n is 1 or 2;
m is 1, 2 or 3;
or a pharmaceutically acceptable acid addition salt, a racemic mixture or its corresponding enantiomer and/or optical isomers thereof.

3. A compound of formula IA according to claim 2, wherein the compound is selected from
6-(4-Chloro-phenyl)-2-[1-(4-hydroxy-3,5-dimethoxy-phenyl)-meth-(Z)-ylidene]-imidazo[2,1-b]thiazol-3-one
6-(4-Chloro-phenyl)-2-[1-(4-hydroxy-3-methoxy-phenyl)-meth-(Z)-ylidene]-imidazo[2,1-b]thiazol-3-one
6-(4-Chloro-phenyl)-2-[1-(3-fluoro-4-hydroxy-5-methoxy-phenyl)-meth-(Z)-ylidene]-imidazo[2,1-b]thiazol-3-one
6-(4-Chloro-phenyl)-2-[1-(3-ethoxy-4-hydroxy-phenyl)-meth-(Z)-ylidene]-imidazo[2,1-b]thiazol-3-one
6-(4-Chloro-phenyl)-2-[1-(3-chloro-4-hydroxy-5-methoxy-phenyl)-meth-(Z)-ylidene]-imidazo[2,1-b]thiazol-3-one
6-(4-Chloro-phenyl)-2-[1-(3-bromo-4-hydroxy-5-methoxy-phenyl)-meth-(Z)-ylidene]-imidazo[2,1-b]thiazol-3-one
6-(4-Chloro-phenyl)-2-[1-(3,4-dihydroxy-5-methoxy-phenyl)-meth-(Z)-ylidene]-imidazo[2,1-b]thiazol-3-one
2-[1-(3-Fluoro-4-hydroxy-5-methoxy-phenyl)-meth-(Z)-ylidene]-6-o-tolyl-imidazo[2,1-b]thiazol-3-one
2-[1-(3-Chloro-4-hydroxy-5-methoxy-phenyl)-meth-(Z)-ylidene]-6-o-tolyl-imidazo[2,1-b]thiazol-3-one
2-[1-(4-hydroxy-3-methoxy-phenyl)-meth-(Z)-ylidene]-6-o-tolyl-imidazo[2,1-b]thiazol-3-one
2-[1-(3-Chloro-4-hydroxy-5-methoxy-phenyl)-meth-(Z)-ylidene]-6-(4-methoxy-phenyl)-5-methyl-imidazo[2,1-b]thiazol-3-one
2-[1-(3-Fluoro-4-hydroxy-5-methoxy-phenyl)-meth-(Z)-ylidene]-6-(4-methoxy-phenyl)-5-methyl-imidazo[2,1-b]thiazol-3-one
2-[1-(4-Hydroxy-3,5-dimethoxy-phenyl)-meth-(Z)-ylidene]-6-(3-trifluoromethoxy-phenyl)-imidazo[2,1-b]thiazol-3-one
2-[1-(4-Hydroxy-3,5-dimethoxy-phenyl)-meth-(Z)-ylidene]-6-(3-trifluoromethyl-phenyl)-imidazo[2,1-b]thiazol-3-one
2-[1-(4-Hydroxy-3,5-dimethoxy-phenyl)-meth-(Z)-ylidene]-6-m-tolyl-imidazo[2,1-b]thiazol-3-one
6-(3-Chloro-phenyl)-2-[1-(4-hydroxy-3,5-dimethoxy-phenyl)-meth-(Z)-ylidene]-imidazo[2,1-b]thiazol-3-one
2-[1-(3-Chloro-4-hydroxy-5-methoxy-phenyl)-meth-(Z)-ylidene]-6-(3-trifluoromethoxy-phenyl)-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(2-fluoro-ethoxy)-ethyl]-N-methyl-benzamide
2-[1-(3-Chloro-4-hydroxy-5-methoxy-phenyl)-meth-(Z)-ylidene]-6-(3-trifluoromethyl-phenyl)-imidazo[2,1-b]thiazol-3-one
6-(3-Chloro-phenyl)-2-[1-(3-chloro-4-hydroxy-5-methoxy-phenyl)-meth-(Z)-ylidene]-imidazo[2,1-b]thiazol-3-one
6-(4-Chloro-phenyl)-2-[1-(3-ethoxy-4-hydroxy-5-methoxy-phenyl)-meth-(Z)-ylidene]-imidazo[2,1-b]thiazol-3-one
6-(4-Chloro-phenyl)-2-[1-(3-(2-fluoro-ethoxy)-4-hydroxy-5-methoxy-phenyl)-meth-(Z)-ylidene]-imidazo[2,1-b]thiazol-3-one
2-[1-(3-Chloro-4-hydroxy-5-methoxy-phenyl)-meth-(Z)-ylidene]-6-(3-fluoro-4-trifluoromethyl-phenyl)-imidazo[2,1-b]thiazol-3-one
2-[1-(3-Hydroxy-3,5-dimethoxy-phenyl)-meth-(Z)-ylidene]-6-(3-fluoro-4-trifluoromethyl-phenyl)-imidazo[2,1-b]thiazol-3-one
6-(4-Chloro-phenyl)-2-[1-(4-hydroxy-3-isopropoxy-5-methoxy-phenyl)-meth-(Z)-ylidene]-imidazo[2,1-b]thiazol-3-one
6-(4-Chloro-phenyl)-2-[1-(4-hydroxy-3-methoxy-5-propoxy-phenyl)-meth-(Z)-ylidene]-imidazo[2,1-b]thiazol-3-one
6-(4-Chloro-phenyl)-2-[1-(4-hydroxy-3-methoxy-5-methyl-phenyl)-meth-(Z)-ylidene]-imidazo[2,1-b]thiazol-3-one
2-[1-(4-Hydroxy-3-methoxy-5-propoxy-phenyl)-meth-(Z)-ylidene]-6-(3-trifluoromethoxy-phenyl)-imidazo[2,1-b]thiazol-3-one
2-[1-(4-Hydroxy-3-methoxy-5-isopropoxy-phenyl)-meth-(Z)-ylidene]-6-(3-trifluoromethoxy-phenyl)-imidazo[2,1-b]thiazol-3-one
2-[1-(3-Fluoro-4-hydroxy-5-methoxy-phenyl)-meth-(Z)-ylidene]-6-(3-trifluoromethoxy-phenyl)-imidazo[2,1-b]thiazol-3-one
2-[1-(4-Hydroxy-3-methoxy-phenyl)-meth-(Z)-ylidene]-6-(3-trifluoromethoxy-phenyl)-imidazo[2,1-b]thiazol-3-one
2-[1-(3-Bromo-4-hydroxy-5-methoxy-phenyl)-meth-(Z)-ylidene]-6-(3-trifluoromethoxy-phenyl)-imidazo[2,1-b]thiazol-3-one
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzonitrile
2-[1-(3-fluoro-4-hydroxy-5-methoxy-phenyl)-meth-(Z)-ylidene]-6-(3-chloro-phenyl)-imidazo[2,1-b]thiazol-3-one
2-[1-(3-Ethyl-4-hydroxy-5-methoxy-phenyl)-meth-(Z)-ylidene]-6-(3-trifluoromethoxy-phenyl)-imidazo[2,1-b]thiazol-3-one
2-[1-(3-fluoro-4-hydroxy-5-methoxy-phenyl)-meth-(Z)-ylidene]-6-(3-(trifluoromethyl)-phenyl)-imidazo[2,1-b]thiazol-3-one
2-[1-(3-fluoro-4-hydroxy-5-methoxy-phenyl)-meth-(Z)-ylidene]-6-(4-fluoro-3-(trifluoromethyl)-phenyl)-imidazo[2,1-b]thiazol-3-one
2-[1-(3-fluoro-4-hydroxy-5-methoxy-phenyl)-meth-(Z)-ylidene]-6-phenyl-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzonitrile
3-{2-[1-(3-Bromo-4-hydroxy-5-methoxy-phenyl)-meth-(Z)-ylidene]-3-oxo-2,3-dihydro-imidazo[2,1-b]thiazol-6-yl}-benzonitrile
2-[1-(3-bromo-4-hydroxy-5-methoxy-phenyl)-meth-(Z)-ylidene]-6-(4-fluoro-3-(trifluoromethyl)-phenyl)-imidazo[2,1-b]thiazol-3-one
2-[1-(3-Fluoro-4-hydroxy-5-methoxy-phenyl)-meth-(Z)-ylidene]-6-(3-methoxy-phenyl)-imidazo[2,1-b]thiazol-3-one
2-[1-(3-Chloro-4-hydroxy-5-methoxy-phenyl)-meth-(Z)-ylidene]-6-(3,5-difluoro-phenyl)-imidazo[2,1-b]thiazol-3-one
3-{2-[1-(4-Hydroxy-3,5-dimethoxy-phenyl)-meth-(Z)-ylidene]-3-oxo-2,3-dihydro-imidazo[2,1-b]thiazol-6-yl}-benzonitrile
3-{2-[1-(4-Hydroxy-3-methoxy-5-isopropoxyphenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-m-tolyl-imidazo[2,1-b]thiazol-3-one
2-[1-(3-Chloro-4-hydroxy-5-methoxy-phenyl)-meth-(Z)-ylidene]-6-(2-fluoro-phenyl)-imidazo[2,1-b]thiazol-3-one
2-[1-(3-Chloro-4-hydroxy-5-methoxy-phenyl)-meth-(Z)-ylidene]-6-(3-ethyl-phenyl)-imidazo[2,1-b]thiazol-3-one
2-[1-(3-Chloro-4-hydroxy-5-methoxy-phenyl)-meth-(Z)-ylidene]-6-(2-methoxy-phenyl)-imidazo[2,1-b]thiazol-3-one
2-[1-(3-Chloro-4-hydroxy-5-methoxy-phenyl)-meth-(Z)-ylidene]-6-(2,5-difluoro-phenyl)-imidazo[2,1-b]thiazol-3-one
2-[1-(3-Chloro-4-hydroxy-5-methoxy-phenyl)-meth-(Z)-ylidene]-6-(3-fluoro-phenyl)-imidazo[2,1-b]thiazol-3-one
2-[1-(3-Fluoro-4-hydroxy-5-methoxy-phenyl)-meth-(Z)-ylidene]-6-(3-fluoro-phenyl)-imidazo[2,1-b]thiazol-3-one
2-[1-(4-Hydroxy-3,5-dimethoxy-phenyl)-meth-(Z)-ylidene]-6-(3-fluoro-phenyl)-imidazo[2,1-b]thiazol-3-one
2-[1-(3-Chloro-4-hydroxy-5-methoxy-phenyl)-meth-(Z)-ylidene]-6-phenyl-imidazo[2,1-b]thiazol-3-one
2-[1-(3-Chloro-4-hydroxy-5-methoxy-phenyl)-meth-(Z)-ylidene]-6-(3-methoxy-phenyl)-imidazo[2,1-b]thiazol-3-one
2-[1-(3-Chloro-4-hydroxy-5-methoxy-phenyl)-meth-(Z)-ylidene]-6-(3-(difluoromethoxy)-phenyl)-imidazo[2,1-b]thiazol-3-one
(Z)-3-(2-(3-fluoro-4-hydroxy-5-methoxybenzylidene)-3-oxo-2,3-dihydroimidazo[2,1-b]thiazol-5-yl)benzonitrile
2-[1-(3-Chloro-4-hydroxy-5-methoxy-phenyl)-meth-(Z)-ylidene]-6-(3-(chloromethyl)phenyl)-imidazo[2,1-b]thiazol-3-one
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-(3-(2-fluoroethoxyl)phenyl)-imidazo[2,1-b]thiazol-3-one
2-[1-(3-Chloro-4-hydroxy-5-methoxy-phenyl)-meth-(Z)-ylidene]-6-(4-(2-fluoroethoxyl)phenyl)-imidazo[2,1-b]thiazol-3-one
2-[1-(3-Chloro-4-hydroxy-5-methoxy-phenyl)-meth-(Z)-ylidene]-6-(2-(2-fluoroethoxyl)phenyl)-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-(4-(difluoromethoxy)phenyl)-imidazo[2,1-b]thiazol-3-one
2-[1-(3-Chloro-4-hydroxy-5-methoxy-phenyl)-meth-(Z)-ylidene]-6-(3-morpholinophenyl)-imidazo[2,1-b]thiazol-3-one
2-[1-(3-Chloro-4-hydroxy-5-methoxy-phenyl)-meth-(Z)-ylidene]-6-(4-(pyrrolidin-1-yl)phenyl)-imidazo[2,1-b]thiazol-3-one
2-[1-(3-Chloro-4-hydroxy-5-methoxy-phenyl)-meth-(Z)-ylidene]-6-(4-(diethylamino)phenyl)-imidazo[2,1-b]thiazol-3-one
2-[1-(3-Chloro-4-hydroxy-5-methoxy-phenyl)-meth-(Z)-ylidene]-6-(4-morpholinophenyl)-imidazo[2,1-b]thiazol-3-one
2-[1-(3-Chloro-4-hydroxy-5-methoxy-phenyl)-meth-(Z)-ylidene]-6-(4-(dimethylamino)phenyl)-imidazo[2,1-b]thiazol-3-one
2-[1-(3-Chloro-4-hydroxy-5-methoxy-phenyl)-meth-(Z)-ylidene]-6-[3-(2-oxo-pyrrolidin-1-yl)-phenyl]-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid
2-[1-(3-Chloro-4-hydroxy-5-methoxy-phenyl)-meth-(Z)-ylidene]-6-(3,4-dimethoxy-phenyl)-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzamide
N-(4-{2-[1-(3-Chloro-4-hydroxy-5-methoxy-phenyl)-meth-(Z)-ylidene]-3-oxo-2,3-dihydro-imidazo[2,1-b]thiazol-6-yl}-phenyl)-acetamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid
2-[1-(3-Chloro-4-hydroxy-5-methoxy-phenyl)-meth-(Z)-ylidene]-6-(2,6-difluoro-phenyl)-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-propyl-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-benzamide
N-Allyl-3-{2-[1-(3-chloro-4-hydroxy-5-methoxy-phenyl)-meth-(Z)-ylidene]-3-oxo-2,3-dihydro-imidazo[2,1-b]thiazol-6-yl}-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-propyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoro-propyl)-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(3-fluoro-phenyl)-ethyl]-N-methyl-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-N-methyl-benzamide
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(2-fluoro-ethyl)-N-methyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N,N-dimethyl-benzamide
2-[1-(3-Chloro-4-hydroxy-5-methoxy-phenyl)-meth-(Z)-ylidene]-6-[4-(4-fluoro-piperidine-1-carbonyl)-phenyl]-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-methyl-N-propyl-benzamide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoropropoxy)-benzamide
2-[1-(3-Chloro-4-hydroxy-5-methoxy-phenyl)-meth-(Z)-ylidene]-6-[4-(3-fluoro-azetidine-1-carbonyl)-phenyl]-imidazo[2,1-b]thiazol-3-one
4-{2-[1-(3-Chloro-4-hydroxy-5-methoxy-phenyl)-meth-(Z)-ylidene]-3-oxo-2,3-dihydro-imidazo[2,1-b]thiazol-6-yl}-N-(3-fluoropropoxy)-N-methyl-benzamide
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-benzoic acid
6-[4-(3-Fluoro-azetidine-1-carbonyl)-phenyl]-2-[1-(3-fluoro-4-hydroxy-5-methoxy-phenyl)-meth-(Z)-ylidene]-imidazo[2,1-b]thiazol-3-one
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoroethoxy)-benzamide
3-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(3-fluoropropoxy)-N-methyl-benzamide
2-[1-(3-Chloro-4-hydroxy-5-methoxy-phenyl)-meth-(Z)-ylidene]-6-[3-(3-fluoro-azetidine-1-carbonyl)-phenyl]-imidazo[2,1-b]thiazol-3-one
2-[1-(3-Chloro-4-hydroxy-5-methoxy-phenyl)-meth-(Z)-ylidene]-6-[3-(4-fluoro-piperidine-1-carbon-phenyl]-imidazo[2,1-b]thiazol-3-one
3-{2-[1-(3-Chloro-4-hydroxy-5-methoxy-phenyl)-meth-(Z)-ylidene]-3-oxo-2,3-dihydro-imidazo[2,1-b]thiazol-6-yl}-N-[2-(2-fluoro-ethoxy)-ethyl]-N-methyl-benz amide
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoroethoxy)-N-methyl-benzamide
4-{2-[1-(3-Fluoro-4-hydroxy-5-methoxy-phenyl)-meth-(Z)-ylidene]-3-oxo-2,3-dihydro-imidazo[2,1-b]thiazol-6-yl}-N-(2-fluoropropoxy)-N-methyl-benz amide
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid 2-fluoro-ethyl ester
4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoic acid 3-fluoro-propyl ester
[2-(4-{2-[1-(3-Chloro-4-hydroxy-5-methoxy-phenyl)-meth-(Z)-ylidene]-3-oxo-2,3-dihydro-imidazo[2,1-b]thiazol-6-yl}-benzoylamino)-ethyl]-carbamic acid tert-butyl ester
N-(2-Amino-ethyl)-4-{2-[1-(3-chloro-4-hydroxy-5-methoxy-phenyl)-meth-(Z)-ylidene]-3-oxo-2,3-dihydro-imidazo[2,1-b]thiazol-6-yl}-benzamide
3-{2-[1-(3-Chloro-4-hydroxy-5-methoxy-phenyl)-meth-(Z)-ylidene]-3-oxo-2,3-dihydro-imidazo[2,1-b]thiazol-6-yl}-benzenesulfonyl fluoride
3-{2-[1-[3-Chloro-5-(3-fluoro-propoxy)-4-hydroxy-phenyl]-meth-(Z)-ylidene]-3-oxo-2,3-dihydro-imidazo[2,1-b]thiazol-6-yl}-benzonitrile
3-Bromo-5-{2-[1-(3-chloro-4-hydroxy-5-methoxy-phenyl)-meth-(Z)-ylidene]-3-oxo-2,3-dihydro-imidazo[2,1-b]thiazol-6-yl}-benzonitrile
2-[1-(3-Chloro-4-hydroxy-5-methoxy-phenyl)-meth-(Z)-ylidene]-6-(3-{2-[2-(2-fluoro-ethoxy)-ethoxy]-ethoxy}-phenyl)-imidazo[2,1-b]thiazol-3-one and
2-[1-(3-Chloro-4-hydroxy-5-methoxy-phenyl)-meth-(Z)-ylidene]-6-(2-{2-[2-(2-fluoro-ethoxy)-ethoxy]-ethoxy}-phenyl)-imidazo[2,1-b]thiazol-3-one.

4. A compound according to claim 1, further defined as a compound of formula IB 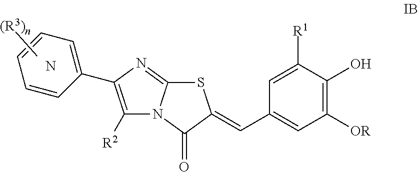
wherein the N-atom of the pyridinyl group may be in different positions, and wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
R³ is hydrogen, halogen, lower alkyl, lower alkyl substituted by halogen, lower alkoxy, lower alkoxy substituted by halogen, O(CH₂)ₘO(CH₂)ₘO-lower alkyl substituted by halogen, cyano, lower alkoxy substituted by hydroxy, lower alkenyloxy, C(O)OH, heterocycloalkyl selected from morpholinyl, pyrrolidinyl or pyrrolidin-2-one, or is heteroaryl selected from imidazolyl substituted by lower alkyl, or is
NR′R″ and R′/R″ are independently from each other hydrogen or lower alkyl or —C(O)-lower alkyl; or is
—C(O)NR⁴R⁵ and
R⁴ is hydrogen or lower alkyl and
R⁵ is hydrogen, lower alkyl, lower alkenyl, —(CH₂)ₘO-lower alkyl substituted by halogen, lower alkyl substituted by halogen, —(CH₂)ₙ-phenyl optionally substituted by halogen, —CH₂)ₘNHC(O)-lower alkyl, or —(CH₂)ₘNH₂, or
R⁴ and R⁵ may form together with the N-atom to which they are attached a piperidine or azetidine ring, which may be substituted by halogen; or is
—C(O)O-lower alkyl substituted by halogen;
n is 1 or 2;
m is 1, 2 or 3;
or a pharmaceutically acceptable acid addition salt, a racemic mixture or its corresponding enantiomer and/or optical isomers thereof.

5. A compound of formula IB according to claim 4, wherein the compound is selected from
2-[1-(4-Hydroxy-3,5-dimethoxy-phenyl)-meth-(Z)-ylidene]-6-pyridin-4-yl-imidazo[2,1-b]thiazol-3-one
2-[1-(4-Hydroxy-3-methoxy-phenyl)-meth-(Z)-ylidene]-6-(2-methyl-pyridin-4-yl)-imidazo[2,1-b]thiazol-3-one
2-[1-(4-Hydroxy-3-methoxy-phenyl)-meth-(Z)-ylidene]-6-(2,6-dimethyl-pyridin-4-yl)-imidazo[2,1-b]thiazol-3-one
2-[1-(4-Hydroxy-3,5-dimethoxy-phenyl)-meth-(Z)-ylidene]-6-(2-methyl-pyridin-4-yl)-imidazo[2,1-b]thiazol-3-one
2-[1-(4-Hydroxy-3,5-dimethoxy-phenyl)-meth-(Z)-ylidene]-6-(2,6-dimethyl-pyridin-4-yl)-imidazo[2,1-b]thiazol-3-one
2-[1-(3-Fluoro-4-hydroxy-5-methoxy-phenyl)-meth-(Z)-ylidene]-6-(6-methoxy-pyridin-2-yl)-imidazo[2,1-b]thiazol-3-one
2-[1-(3-Chloro-4-hydroxy-5-methoxy-phenyl)-meth-(Z)-ylidene]-6-(2,6-dimethyl-pyridin-4-yl)-imidazo[2,1-b]thiazol-3-one
2-[1-(3-Fluoro-4-hydroxy-5-methoxy-phenyl)-meth-(Z)-ylidene]-6-(2,6-dimethyl-pyridin-4-yl)-imidazo[2,1-b]thiazol-3-one
2-[1-(3-Bromo-4-hydroxy-5-methoxy-phenyl)-meth-(Z)-ylidene]-6-(2,6-dimethyl-pyridin-4-yl)-imidazo[2,1-b]thiazol-3-one
2-[1-(3-Chloro-4-hydroxy-5-methoxy-phenyl)-meth-(Z)-ylidene]-6-(pyridin-4-yl)-imidazo[2,1-b]thiazol-3-one
2-[1-(3-Fluoro-4-hydroxy-5-methoxy-phenyl)-meth-(Z)-ylidene]-6-(pyridin-4-yl)-imidazo[2,1-b]thiazol-3-one
2-[1-(3-Bromo-4-hydroxy-5-methoxy-phenyl)-meth-(Z)-ylidene]-6-(pyridin-4-yl)-imidazo[2,1-b]thiazol-3-one
2-[1-(3-Fluoro-4-hydroxy-5-methoxy-phenyl)-meth-(Z)-ylidene]-6-(pyridin-3-yl)-imidazo[2,1-b]thiazol-3-one
2-[1-(3-Bromo-4-hydroxy-5-methoxy-phenyl)-meth-(Z)-ylidene]-6-(pyridin-3-yl)-imidazo[2,1-b]thiazol-3-one
2-[1-(3-Chloro-4-hydroxy-5-methoxy-phenyl)-meth-(Z)-ylidene]-6-(2-methylpyridin-4-yl)-imidazo[2,1-b]thiazol-3-one
2-[1-(3-Fluoro-4-hydroxy-5-methoxy-phenyl)-meth-(Z)-ylidene]-6-(2-methylpyridin-4-yl)-imidazo[2,1-b]thiazol-3-one
2-[1-(3-Bromo-4-hydroxy-5-methoxy-phenyl)-meth-(Z)-ylidene]-6-(2-methylpyridin-4-yl)-imidazo[2,1-b]thiazol-3-one
2-[1-(3-Chloro-4-hydroxy-5-methoxy-phenyl)-meth-(Z)-ylidene]-6-(pyridin-3-yl)-imidazo[2,1-b]thiazol-3-one
2-[1-(3-Chloro-4-hydroxy-5-methoxy-phenyl)-meth-(Z)-ylidene]-6-[6-(4-methyl-3H-imidazol-1-yl)-pyridin-3-yl]-imidazo[2,1-b]thiazol-3-one
2-[1-(3-Chloro-4-hydroxy-5-propoxy-phenyl)-meth-(Z)-ylidene]-6-(4-methyl-pyridin-3-yl)-imidazo[2,1-b]thiazol-3-one
2-[1-(3-Chloro-4-hydroxy-5-propoxy-phenyl)-meth-(Z)-ylidene]-6-pyridin-3-yl-imidazo[2,1-b]thiazol-3-one
2-[1-(3-Chloro-4-hydroxy-5-propoxy-phenyl)-meth-(Z)-ylidene]-6-pyridin-4-yl-imidazo[2,1-b]thiazol-3-one
2-[1-(3-Chloro-4-hydroxy-5-propoxy-phenyl)-meth-(Z)-ylidene]-6-[6-(4-methyl-imidazol-1-yl)-pyridin-3-yl]-imidazo[2,1-b]thiazol-3-one
2-[1-(3-Fluoro-4-hydroxy-5-propoxy-phenyl)-meth-(Z)-ylidene]-6-pyridin-4-yl-imidazo[2,1-b]thiazol-3-one
2-[1-(3-Chloro-5-(3-fluoropropoxy)-4-hydroxy-phenyl)-meth-(Z)-ylidene]-6-pyridin-4-yl-imidazo[2,1-b]thiazol-3-one
2-[1-(3-Fluoro-5-(3-fluoropropoxy)-4-hydroxy-phenyl)-meth-(Z)-ylidene]-6-pyridin-4-yl-imidazo[2,1-b]thiazol-3-one
2-[1-(3-Chloro-4-hydroxy-5-propoxy-phenyl)-meth-(Z)-ylidene]-6-pyridin-2-yl-imidazo[2,1-b]thiazol-3-one
2-[1-(3-Chloro-4-hydroxy-5-methoxy-phenyl)-meth-(Z)-ylidene]-6-(2-chloro-pyridin-4-yl)-imidazo[2,1-b]thiazol-3-one and
2-[1-[3-Chloro-5-(3-fluoro-propoxy)-4-hydroxy-phenyl]-meth-(Z)-ylidene]-6-(2,6-dimethyl-pyridin-4-yl)-imidazo[2,1-b]thiazol-3-one.

6. A compound according to claim 1, further defined as a compound of formula IC 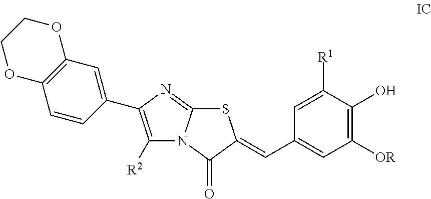
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

7. A compound of formula IC according to claim 6, wherein the compound is 2-[1-(3-Chloro-4-hydroxy-5-methoxy-phenyl)-meth-(Z)-ylidene]-6-(2,3-dihydro-benzo[1,4]dioxin-6-yl)-imidazo[2,1-b]thiazol-3-one.

8. A compound according to claim 1, further defined as a compound of formula ID 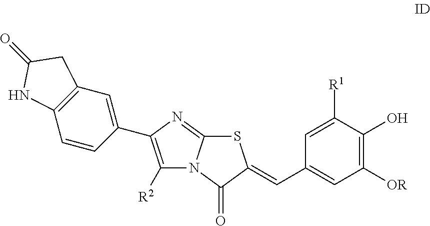
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

9. A compound of formula ID according to claim 8, wherein the compound is 5-{2-[1-(3-Chloro-4-hydroxy-5-methoxy-phenyl)-meth-(Z)-ylidene]-3-oxo-2,3-dihydro-imidazo[2,1-b]thiazol-6-yl}-1,3-dihydro-indol-2-one.

10. A compound according to claim 1, further defined as a compound of formula IE 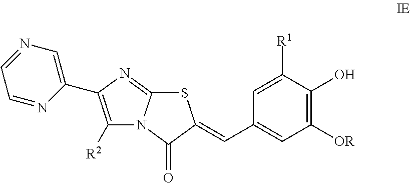
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

11. A compound of formula IE according to claim 10, wherein the compound is 2-[1-(3-Chloro-4-hydroxy-5-propoxy-phenyl)-meth-(Z)-ylidene]-6-pyrazin-2-yl-imidazo[2,1-b]thiazol-3-one.

12. A compound according to claim 1, further defined as a compound of formula IF 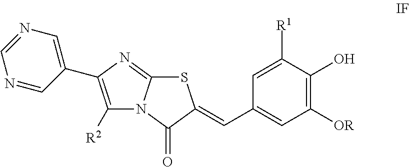
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

13. A compound of formula IF according to claim 12, wherein the compound is 2-[1-(3-Chloro-4-hydroxy-5-propoxy-phenyl)-meth-(Z)-ylidene]-6-pyrimidin-5-yl-imidazo[2,1-b]thiazol-3-one.

14. A compound according to claim 1, further defined as a compound of formula IG 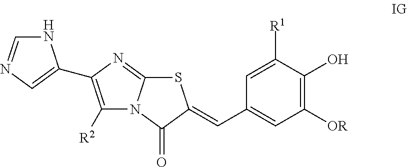
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

15. A compound of formula IG according to claim 14, wherein the compound is 2-[1-(3-Chloro-4-hydroxy-5-propoxy-phenyl)-meth-(Z)-ylidene]-6-(3H-imidazol-4-yl)-imidazo[2,1-b]thiazol-3-one.

16. A compound according to claim 1, further defined as a compound of formula IH 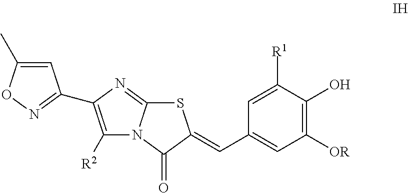
wherein
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

17. A compound of formula IH according claim 16, wherein the compound is 2-[1-(3-Chloro-4-hydroxy-5-methoxy-phenyl)-meth-(Z)-ylidene]-6-(5-methyl-isoxazol-3-yl)-imidazo[2,1-b]thiazol-3-one.

18. A compound according to claim 1, further defined as a compound of formula IJ 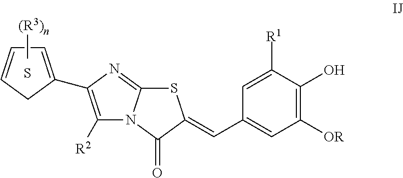
wherein
R³ is hydrogen or lower alkyl
n is 1 or 2
R is lower alkyl or lower alkyl substituted by halogen;
R¹ is hydrogen, halogen, hydroxy, lower alkoxy, lower alkyl and lower alkoxy substituted by halogen;
R² is hydrogen, lower alkyl;
or a pharmaceutically acceptable acid addition salt, a racemic mixture or its corresponding enantiomer and/or optical isomers thereof.

19. A compound of formula IJ according to claim 18, wherein the compound is selected from
2-[1-(3-Chloro-4-hydroxy-5-propoxy-phenyl)-meth-(Z)-ylidene]-6-thiophen-3-yl-imidazo[2,1-b]thiazol-3-one
2-[1-(3-Chloro-4-hydroxy-5-propoxy-phenyl)-meth-(Z)-ylidene]-6-thiophen-2-yl-imidazo[2,1-b]thiazol-3-one and
2-[1-(3-Chloro-4-hydroxy-5-propoxy-phenyl)-meth-(Z)-ylidene]-6-(2,4-dimethylthiophen-3-yl)-imidazo[2,1-b]thiazol-3-one.

20. A method of binding and imaging tau aggregates, beta-amyloid aggregates or alpha-synuclein aggregates, comprising contacting the aggregate with a compound according to claim 1.

21. The method of claim 20, wherein the aggregate is comprised in an Alzheimer's patient.

22. A pharmaceutical composition comprising a compound of formula I according to claim 1 and a pharmaceutical acceptable carrier.

23. A method of imaging tau-aggregate deposits, comprising
introducing into a mammal a detectable quantity of a composition according to claim 22;
allowing sufficient time for the compound of formula I to be associated with tau-aggregate deposit, and
detecting the compound associated with one or more tau-aggregate deposits.
